(19) United States
(12) Patent Application Publication
(10) Pub. No.: US 2025/0289879 A1
(43) Pub. Date: Sep. 18, 2025

(54) THERAPEUTIC MODULATION OF GENES IN MYELOID LINEAGE CELLS AND USES THEREOF IN OPHTHALMOLOGY

(71) Applicant: The Children's Medical Center Corporation, Boston, MA (US)

(72) Inventors: Ye Sun, Chestnut Hill, MA (US); Tianxi Wang, Brookline, MA (US)

(73) Assignee: The Children's Medical Center Corporation, Boston, MA (US)

(21) Appl. No.: 18/861,150

(22) PCT Filed: Apr. 28, 2023
(86) PCT No.: PCT/US2023/066379

§ 371 Date: Oct. 28, 2024

Related U.S. Application Data
(60) Provisional application No. 63/336,975, filed on Apr. 29, 2022.

(51) Int. Cl. C07K 16/24; A61K 45/06; A61P 27/02; C12N 15/86

(52) U.S. Cl. CPC C07K 16/24; A61K 45/06; A61P 27/02; C12N 15/86; C07K 2317/76; C12N 2750/14143

(57) ABSTRACT

Provided herein are methods for reducing the activation of myeloid lineage cells of a subject by targeting the expression and/or activity of factors that contribute to myeloid cell activation with a therapeutic agent. As a result of these methods, the activity of myeloid lineage cells that contribute to neovascularization in the retina of a subject may be reduced. The methods are useful for the treatment and/or prevention of various retinal diseases and injuries, including retinopathy of prematurity (ROP), age-related macular degeneration (AMD), retinitis pigmentosa, or diabetic retinopathy.

### RELATED APPLICATION

This application claims the benefit under 35 U.S.C. § 119 (c) to U.S. Provisional Application No. 63/336,975, entitled “THERAPEUTIC MODULATION OF GENES IN MYELOID LINEAGE CELLS AND USES THEREOF IN OPHTHALMOLOGY,” filed on Apr. 29, 2022, the entire contents of which are incorporated herein by reference.

### GOVERNMENT SUPPORT

This invention was made with government support under Grant numbers EY030140. EY029238, HL146128, awarded by The National Institutes of Health. The government has certain rights in the invention.

### REFERENCE TO AN ELECTRONIC SEQUENCE LISTING

The contents of the electronic sequence listing (C123370246WO00-SEQ-VLJ.xml; Size: 20,008 bytes; and Date of Creation: Apr. 27, 2023) is herein incorporated by reference in its entirety.

### BACKGROUND

Diseases that affect the retina, broadly referred to as retinopathies, can occur as a result of both genetic (e.g., retinitis pigmentosa) and environmental (e.g., retinopathy of prematurity (ROP)) influences, and are generally characterized by a progressive loss of visual acuity, eventually leading to blindness. During retinopathies, damage to the retina causes an inflammatory response during which myeloid lineage immune cells infiltrate the retina. These cells secrete a variety of cytokines and chemokines that promote the formation of new blood vessels in the retina (neovascularization), which is a major contributing factor for vision loss. Although suppressor of cytokine signaling 3 (SOCS3) has been shown to have a protective effect on the retina by suppressing the release of cytokines, it is poorly understood which factors expressed by myeloid lineage cells drive myeloid cell activation and promote retinal neovascularization.

### SUMMARY

The present disclosure is based on the discovery of certain factors which are induced in myeloid lineage cells of subjects having or at risk for a disease affecting the retina, such as, for example, retinopathy of prematurity (ROP), age-related macular degeneration (AMD), retinitis pigmentosa, or diabetic retinopathy. These factors are found to contribute to activation of these myeloid lineage cells, which in turn causes neovascularization of the retina and progressive vision loss that can lead to blindness. Accordingly, some aspects of the present disclosure relate to a method of reducing activation of myeloid lineage cells in a subject, comprising administering to a subject in need thereof an effective amount of an agent that results in a decrease in the expression and/or activity of a factor associated with myeloid cell activation.

In some embodiments, the factor associated with myeloid cell activation is selected from the group consisting of secreted phosphoprotein 1 (Spp1), triggering receptor expressed on myeloid cells 2 (Trem2), translocator protein (Tspo), and signal transducer and activator of transcription 3 (STAT3), or a combination thereof.

In some embodiments, the agent inhibits expression of the factor associated with myeloid cell activation. In some embodiments, the agent comprises a small interfering RNA (siRNA), a short hairpin RNA (shRNA), or an antisense oligonucleotide (ASO) that is complementary to a gene encoding the factor associated with myeloid cell activation. In some embodiments, the agent comprises a viral vector that encodes a siRNA or a shRNA that is complementary to a gene encoding the factor associated with myeloid cell activation. In some embodiments, the viral vector is a lentiviral vector or a recombinant adeno-associated viral vector (rAAV). In some embodiments, the siRNA, shRNA, or ASO is at least 60%, at least 70%, at least 80%, at least 90%, at least 95%, at least 99%, or 100% complementary to a region of the gene encoding the factor associated with myeloid cell activation.

In some embodiments, the agent inhibits the activity of the factor associated with myeloid cell activation. In some embodiments, the agent is a protein, a peptide, an aptamer, or a small molecule. In some embodiment, the agent binds to the factor associated with myeloid cell activation. In some embodiments, the agent is an antibody that is specific for the factor associated with myeloid cell activation.

In some embodiments, the method further comprises administering to the subject a second agent that results in an increase in the expression and/or activity of suppressor of cytokine signaling 3 (SOCS3).

In some embodiments, the method further comprises administering to the subject a second agent that results in a decrease in the expression and/or activity of cellular Fos proto-oncogene (c-Fos) or vascular endothelial growth factor A (VEGFA).

In some embodiments, the second agent is a RNA, a protein, a peptide, an aptamer, or a small molecule.

In some embodiments, the administration results in a decrease in the expression and/or activity of the factor associated with myeloid cell activation by up to 10%, up to 20%, up to 30%, up to 40%, up to 50%, up to 60%, up to 70%, up to 80%, up to 90%, up to 95%, up to 99%, or up to 100%, as compared to expression and/or activity of the factor associated with myeloid cell activation prior to the administration.

In some embodiments, the expression and/or activity of the factor associated with myeloid cell activation is decreased in myeloid lineage cells of the subject.

In some embodiments, the administration results in reduced activation of myeloid lineage cells in the subject by up to 10%, up to 20%, up to 30%, up to 40%, up to 50%, up to 60%, up to 70%, up to 80%, up to 90%, up to 95%, up to 99%, or up to 100%, as compared to myeloid lineage cell activation in the absence of the administration. In some embodiments, the activation of myeloid lineage cells comprises proliferation of myeloid lineage cells in the subject.

In some embodiments, the myeloid lineage cells are myeloid lineage cells occurring in the retina of the subject.

In some embodiments, the myeloid lineage cells are microglial cells, macrophages, B cells, T cells, monocytes, or granulocytes.

In some embodiments, the myeloid lineage cells are myeloid lineage cells in which expression and/or activity of Spp1, Trem2, Tspo, and/or STAT3 is upregulated prior to administration.

In some embodiments, the myeloid lineage cells are myeloid lineage cells in which expression and/or activity of SOCS3 is downregulated prior to administration.

In some embodiments, the administration results in reduced neovascularization in the retina of the subject by up to 10%, up to 20%, up to 30%, up to 40%, up to 50%, up to 60%, up to 70%, up to 80%, up to 90%, up to 95%, up to 99%, or up to 100%, as compared to neovascularization in the retina of the subject in the absence of the administration.

In some embodiments, the subject is a mammalian subject. In some embodiments, the subject is a human.

In some embodiments, the subject has, is suspected of having, or is at risk of developing a retinopathy, a retinal injury, or a glaucoma associated with retinopathy. In some embodiments, the retinopathy is retinopathy of prematurity (ROP), dry age-related macular degeneration (AMD), wet AMD, retinal degeneration, retinitis pigmentosa, or diabetic retinopathy.

In some embodiments, the administration treats the retinopathy, retinal injury, or glaucoma associated with retinopathy in the subject. In some embodiments, the administration prevents the development of the retinopathy, retinal injury, or glaucoma associated with retinopathy in the subject.

In some aspects, the present disclosure further relates to a method of treating a retinopathy, a retinal injury, or a glaucoma associated with retinopathy in a subject, comprising collecting myeloid lineage cells from the subject in need thereof, treating the myeloid lineage cells with an agent that results in a decrease in the expression and/or activity of a factor associated with myeloid cell activation, and administering the myeloid lineage cells to the subject, thereby treating a retinopathy, a retinal injury, or a glaucoma associated with retinopathy in the subject.

In some embodiments, the factor associated with myeloid cell activation is selected from the group consisting of secreted phosphoprotein 1 (Spp1), triggering receptor expressed on myeloid cells 2 (Trem2), translocator protein (Tspo), and signal transducer and activator of transcription 3 (STAT3), or a combination thereof.

In some embodiments, the agent inhibits expression of the factor associated with myeloid cell activation. In some embodiments, the agent comprises a small interfering RNA (siRNA), a short hairpin RNA (shRNA), or an antisense oligonucleotide (ASO) that is complementary to a gene encoding the factor associated with myeloid cell activation. In some embodiments, the agent comprises a viral vector that encodes a siRNA or a shRNA that is complementary to a gene encoding the factor associated with myeloid cell activation. In some embodiments, the viral vector is a lentiviral vector or a recombinant adeno-associated viral vector (rAAV). In some embodiments, the siRNA, shRNA, or ASO is at least 60%, at least 70%, at least 80%, at least 90%, at least 95%, at least 99%, or 100% complementary to a region of the gene encoding the factor associated with myeloid cell activation.

In some embodiments, the agent inhibits the activity of the factor associated with myeloid cell activation. In some embodiments, the agent is a protein, a peptide, an aptamer, or a small molecule. In some embodiments, the agent binds to the factor associated with myeloid cell activation. In some embodiments, the agent is an antibody that is specific for the factor associated with myeloid cell activation.

In some embodiments, the method further comprises treating the myeloid lineage cells with a second agent that results in an increase in the expression and/or activity of suppressor of cytokine signaling 3 (SOCS3).

In some embodiments, the method further comprises treating the myeloid lineage cells with a second agent that results in a decrease in the expression and/or activity of cellular Fos proto-oncogene (c-Fos) or vascular endothelial growth factor A (VEGFA).

In some embodiments, the second agent is a RNA, a protein, a peptide, an aptamer, or a small molecule.

In some embodiments, the treatment results in a decrease in the expression and/or activity of the factor associated with myeloid cell activation in the myeloid lineage cells by up to 10%, up to 20%, up to 30%, up to 40%, up to 50%, up to 60%, up to 70%, up to 80%, up to 90%, up to 95%, up to 99%, or up to 100%, as compared to expression and/or activity of the factor associated with myeloid cell activation prior to the treatment.

In some embodiments, the treatment results in reduced activation of the myeloid lineage cells by up to 10%, up to 20%, up to 30%, up to 40%, up to 50%, up to 60%, up to 70%, up to 80%, up to 90%, up to 95%, up to 99%, or up to 100%, as compared activation of the myeloid lineage cells prior to the treatment. In some embodiments, the activation of the myeloid lineage cells comprises proliferation of the myeloid lineage cells.

In some embodiments, the myeloid lineage cells are microglial cells, macrophages, B cells, T cells, monocytes, or granulocytes.

In some embodiments, the myeloid lineage cells are collected from bone marrow of the subject.

In some embodiments, the myeloid lineage cells are administered to the retina of the subject.

In some embodiments, the administration results in reduced neovascularization in the retina of the subject by up to 10%, up to 20%, up to 30%, up to 40%, up to 50%, up to 60%, up to 70%, up to 80%, up to 90%, up to 95%, up to 99%, or up to 100%, as compared to neovascularization in the retina of the subject in the absence of the administration.

In some embodiments, the subject is a mammalian subject. In some embodiments, the subject is a human.

In some embodiments, the retinopathy is retinopathy of prematurity (ROP), dry age-related macular degeneration (AMD), wet AMD, retinal degeneration, retinitis pigmentosa, or diabetic retinopathy.

### BRIEF DESCRIPTION OF DRAWINGS

The accompanying drawings are not intended to be drawn to scale. In the drawings, each identical or nearly identical component that is illustrated in various FIGs. is represented by a like numeral. For purposes of clarity, not every component may be labeled in every drawing.

In the drawings:

FIGS. 1A-1H show how myeloid cells were accumulated during the development of OIR. FIG. 1A shows the development of a myeloid lineage-specific reporter mouse model, in which myeloid cells and non-myeloid cells are alternately labeled with fluorescent reporters. FIGS. 1B-1E show a visualization of the immune-vascular network during OIR using high-resolution two-photon microscopy in the intact retina from myeloid-specific mTmG reporter mice (Cre^{LysM}::mTmG). FIG. 1B is a schematic anatomical representation of a section through the mouse eye depicting retina layer and dissected retinal cup mounted for two-photon imaging. FIG. 1C shows representative 3D-rendered two-photon images of the outer and underside layers of the mouse retina from Cre^{LysM}::mTmG P17 mice. The tdTomato marker was used to identify LysM-(non-myeloid) cells, the GFP marker was used to identify myeloid cells, and vascular tree (Qtracker655/CD31) are shown. FIGS. 1D-1E are zoomed images from (FIG. 1C) depicting myeloid cell and blood vessel interactions at P17 OIR in Cre^{LysM}::mTmG mice. Scale bar, 50 μm. FIGS. 1F-1G are confocal images of mouse retinas from Cre^{LysM}::mTmG P17 OIR mice. FIG. 1F shows how GFP⁺ myeloid cells were around the NV area labelled by endothelial cell marker CD31. FIG. 1G shows how the round shape GFP⁺ myeloid cells were co-stained with active microglia and macrophage marker IBA1. FIG. 1H shows how IBA1⁺ myeloid cells were increased in the NV areas in OIR retinas compared to normal retinas. Scale bar: 100 μm in (FIG. 1A), 1 unit=102.6 μm in (FIG. 1C), 20 μm in (FIG. 1D), 10 μm in (FIG. 1E), 200 μm in (FIG. 1F), and 40 μm in (FIG. 1G).

FIGS. 2A-2F show single cell sequencing of CD45+ myeloid immune cells from normal retina and OIR retina. FIG. 2A shows aberrant blood vessel formation in retina from OIR mice, as compared to retina from mice without OIR. FIG. 2B shows a schematic illustrating a workflow for single cell RNA sequencing of CD45+ myeloid immune cells from retina. FIG. 2C shows a t-distributed stochastic neighbor embedding (t-SNE) analysis of myeloid immune cells subjected to single cell RNA sequencing. FIGS. 2D and 2E show clustering and identification of sequenced myeloid cells on the basis of expressed myeloid cell biomarkers. FIG. 2E shows how expression of principal hematopoietic lineage markers was mapped into different immune cell types using ImmGen ULI RNA-Seq database. FIG. 2F shows identification of cells shown in FIG. 2C according to identified cell type.

FIGS. 3A-3B show immune cell profile in healthy retina and retina with OIR. FIG. 3A shows differing cell type profiles of myeloid lineage cells in retina from mice with OIR as compared to retina from mice without OIR. FIG. 3B shows quantification of data shown in FIG. 3A.

FIGS. 4A-4K show how SOCS3 was induced in myeloid cells and controlled NV in OIR retina. FIG. 4A shows increased SOCS3 expression in macrophages and microglia during OIR. FIG. 4B shows how SOCS3 expression was increased in OIR retina and colocalized with CD11b+ myeloid cells in OIR retina at P14 compared to normal retinal (n=6). FIGS. 4C-4D show how loss of myeloid Socs3 (Socs3 cKO) dramatically increased retinal NV at P17 in OIR retinas compared to littermate floxed control retinas (Socs3 f/f) (n=28-34). FIGS. 4E-4F show how overexpression of myeloid Socs3 (Socs3 COE) significantly reduced NV at P17 in OIR retinas compared to littermate floxed controls (Socs3 OE). VO was not influenced by loss or overexpression of Socs3 (n=20-22). FIG. 4G shows how worsened vessel tortuous was observed in Socs3 cKO mice compared to littermate floxed control mice (n=4). FIG. 4H show that loss of SOCS3 during OIR is associated with increased retinal blood vessel tortuosity and integrity. FIGS. 4I and 4J show that loss of SOCS3 during OIR is associated with decreased visual function. FIG. 4K shows how myeloid-specific LysM-Cre driven-Ai9 tdTomato reporter mice with OIR, myeloid lineage cells (tdTomato) were increased in OIR retina and SOCS3 was induced in those myeloid cells (tdTomato) compared to normal controls at P17. The morphology of myeloid lineage cells was changed from ramified shape in normal retinas to activated or amocboid shapes in OIR retinas (n=6). **, p<0.01; ***, p<0.001; n.s., no significance. Scale bar: 200 μm in (FIG. 4B), Scale bar: 1000 μm in (FIG. 4K) first line, Scale bar: 200 μm in (FIG. 4K) second line, Scale bar: 500 μm in (FIG. 4C), and Scale bar: 1000 μm in (FIG. 4E).

FIGS. 5A-5D show cell clusters of myeloid lineage cells and induction of novel genes in myeloid cells during OIR without SOCS3 function. FIG. 5A shows clustering and identification of sequenced myeloid cells on the basis of expressed myeloid cell biomarkers during OIR without SOCS3 function. FIG. 5B shows quantification of data shown in FIG. 5A. FIG. 5C shows an increase in microglia and macrophages in retina during OIR with progressive loss of SOCS3 function. FIG. 5D shows the upregulation of downregulation of genes controlled by SOCS3 during OIR.

FIGS. 6A-6E show that Spp1 is induced in retina during OIR without SOCS3 function. FIG. 6A shows Spp1-expressing cells were increased in NVAMs in OIR and s-OIR retinas showing by tSNE plots. FIG. 6B shows quantification of data shown in FIG. 6A. FIG. 6C shows increased Spp1 expression in OIR with progressive loss of SOCS3 function in microglia and macrophages. FIG. 6D shows significantly increased Spp1 expression in the absence of SOCS3 function by quantitative polymerase chain reaction (qPCR). FIG. 6E shows SPP1 expression was induced in IBA1⁺ cells in the retinas with OIR and further increased on the retinas with s-OIR at P13 and P14 (n=6).

FIGS. 7A-7C show Spp1 is a direct target of STAT3. FIG. 7A shows representative genome browser tracks of Chip-Seq data in murine bone marrow-derived macrophage (BMDM) cells stimulated with IL-10, LPS or IL-10 and LPS (GSE86169) and the enhancer maker H3K27ac Chip-seq data in BMDM cells with no stimulation (GSE188145) for the Spp1 gene. FIG. 7B shows a graph of a Spp1 promoter assay measured by relative luciferase assay (fold change) in control, P2.6K, E1, and E2. FIG. 7C shows a schematic for a luciferase promotor and enhancer assay to determine whether Spp1 is transcriptionally regulated by STAT3. FIG. 7B shows that a fragment of Spp1 (P2.6k) is transcriptionally regulated by STAT3. FIG. 7C shows a dose dependent response of Spp1 transcription in the presence of varying quantities of STAT3.

FIGS. 8A-8C show that reduced Spp1 activity is associated with reduced neovascularization and blood vessel loss. FIG. 8A shows a schematic for inducing OIR in mice and treating with an antibody that blocks Spp1 function. FIG. 8B shows that administration of an anti-Spp1 antibody reduces neovascularization. FIG. 8C shows that administration of an anti-Spp1 antibody reduces blood vessel loss.

FIGS. 9A-9C show induction of Trem2 during OIR, especially during loss of SOCS3 function. FIG. 9A shows induction of Trem2 during OIR in macrophages. FIGS. 9B and 9C show induction of Trem2 during OIR in microglia.

FIGS. 10A-10C show induction of Tspo during OIR, especially during loss of SOCS3 function. FIG. 10A shows induction of Tspo during OIR in macrophages. FIGS. 10B and 10C show induction of Tspo during OIR in microglia.

FIGS. 11A-11F show the distribution of immune cell types in retina were altered during OIR. FIGS. 11A-11B show a schematic representation of retinal flat mount images for normal retinas and OIR retinas as well as the workflow for scRNA-seq. FIG. 11C shows single-cell transcriptomes of immune cells from all three groups were analyzed and 11 distinct clusters of cells were identified. FIG. 11D shows how expression of principal hematopoietic lineage markers in the 11 identified cell clusters was shown as percent expressed and average expression. FIG. 11E shows how 11 clusters were identified in all three groups: normal retina, OIR retina, and s-OIR. FIG. 11F shows how the percentage of total CD45⁺ cells in each cluster in normal retinas, OIR retinas, and s-OIR were calculated and the cell numbers in each cluster were compared using c2 Test. s-OIR: myeloid SOCS3 deficient mice with OIR. *, p<0.05, **, p<0.01, ***, p<0.001. ****, p<0.0001, ns, no significance.

FIGS. 12A-12E show how NVAMs were accumulated during OIR via SOCS3 control. FIGS. 12A-12B show how GOBP analysis on pathways involved in macrophage differentiation and phagocytosis in normal retinas, OIR retinas, and s-OIR reinas. FIG. 12C shows the distribution of microglia 1, 2, 3, and macrophage 2 in total immune cells in normal retinas, OIR retinas, and s-OIR retinas showing by tSNE plot. FIG. 12D shows how RNA Velocity analysis was projected on ForceAtlas 2 (FA) embedding to represent the cell fate transitions of microglia 1, 2, 3, and macrophage 2 in normal retinas, OIR retinas, and s-OIR retinas. (FIG. 12E) Pseudotime analysis in normal retinas, OIR retinas, and s-OIR retinas. Latent time was calculated using scVelo's latent time function and early cell types (microglia 1) were assigned early time points, where mature cells (macrophage 1 and 2) were associated with later ones. s-OIR: myeloid SOCS3 deficient mice with OIR.

FIGS. 13A-13D show how SPP1 was highly induced in NVAMs in myeloid SOCS3 deficient OIR retinas. FIG. 13A shows how top DEGs expressed in NVMAs in retinas with s-OIR vs OIR were analyzed. FIG. 13B show how Spp1-expressing cells were increased in NVAMs in OIR and s-OIR retinas showing by ForceAtlas2 (FA) plots. FIG. 13C shows how the mRNA levels of Spp1 were increased in CD45⁺ cells from OIR retinas and further increased in s-OIR retinas at P13 and P14 (n=6). FIG. 13D shows how SPP1 expression was induced in microglia in laser-induced CNV and myeloid Socs3 deficient-induced CNV (s-CNV) (n=6). Scale bar: 200 μm in (FIG. 6E) and 1000 μm in (FIG. 13D). s-OIR: myeloid SOCS3 deficient mice with OIR.

FIGS. 14A-14D show how Spp1 was the direct target of STAT3. FIG. 14A shows representative genome browser tracks using the Integrative Genomics Viewer of the STAT3 Chip-Seq data in murine bone marrow-derived macrophage (BMDM) cells stimulated with IL-10 and LPS (GSE86169) and the enhancer maker H3K27ac Chip-seq data in BMDM cells with no stimulation (GSE188145) for the Spp1 gene. The dots showed the location of the STAT3 binding motif. The rectangles indicate the region used for luciferase reporter assay. FIG. 14B is a schematic of the luciferase reporters including Spp1 promoter or enhancer regions. The 5′ region of the Spp1 gene from TSS to −2.6 k bp (P2.6k), from −6.5 k bp to −7.0 k bp (E1), and from −7.5 k bp to −9.0 k bp (E2) were subcloned to the luciferase reporter pTA-luc plasmid. The gray circles indicate putative Stat3 binding sites. FIG. 14C shows how the reporter activity of the promoter and enhancers of the Spp1 gene was measured by the luciferase assay. The relative luciferase activity was shown as the ratio of luminescence from the reporter to that of a control (empty pTA-luc vector). Luciferase reporter activity was induced by Promoter 2.6K, but not Enhancer 1 and 2 (n=6). FIG. 14D shows how STAT3 binding sites of P2.6k were mutated from TTCNNNGAA to gTgNNNcAc. Luciferase reporters with native (P2.6k) or mutated STAT3 (P2.6k-Mut) binding sites in Spp1 promoter were cloned and cotransfected with STAT3-expressing vector. STAT3 dose-dependently increased p2.6k, but not P2.6k-Mut luciferase expression in pTA-luc vector (n=6-8). *, p<0.05; **, p<0.01; ***, p<0.001; n.s., no significance.

FIGS. 15A-15F show how deletion of SPP1 suppressed myeloid SOCS3-induced NV in two mouse models. FIG. 15A shows the hypotheses for the Spp1 and myeloid SOCS3 double knockout mouse phenotype. FIGS. 15B-15C show how deletion of myeloid Socs3 in Spp1 knockout background attenuated myeloid SOCS3 loss-induced NV in OIR mice (n=14-18 retinas) and in laser-induced CNV mice (n=24 lesions) (FIGS. 15D-15E). FIG. 15F shows how SOCS3/SPP1 pathway controlled Immune-Vascular crosstalk in proliferative retinopathy. n.s., no significance. Scale bar: 1000 μm in (FIG. 15B) and 500 μm in (FIG. 15D).

FIGS. 16A-16G show how SOCS3 agonists and SPP1 neutralizing antibody inhibited NV in OIR retinas. FIGS. 16A-16B show how SOCS3 agonist. naringenin treatment (i.p.) from P12 to P16 significantly reduced NV at P17 compared to control treatment without influencing VO (n=10 retinas). FIGS. 16C-16E show how SOCS3 peptide mimic intravitreal injection at P12 inhibited NV compared to control peptide treatment (n=8 retinas). FIG. 16C shows SEQ ID NO: 19 (top) and SEQ ID NO: 20 (bottom). FIGS. 16F-16G show how SPP1 neutralizing antibody treatment at P14 reduced NV in both OIR retinas and s-OIR retinas without influencing VO (n=6-9 retinas). Scale bar. 1000 μm in FIG. 16A. FIG. 16D. and FIG. 16F. s-OIR: myeloid SOCS3 deficient mice with OIR.

FIG. 17 shows how SPP1 was induced in CNV areas in laser-induced CNV mice. SPP1 was induced at day 1 post laser exposure and continued to increase at day 3 post laser exposure in a laser-induced CNV model (n=6-8 retinas). Scale bar: 500 μm.

FIG. 18 presents Feature ForceAtlas2 plots showing the reduced levels of microglia markers, Cx3cr1 and Trem119 expression in OIR and myeloid SOCS3 deficient OIR (s-OIR) compared to normal retinas.

FIGS. 19A-19B show CellChat pathway analysis of scRNA-seq in normal retina and OIR retinas. FIG. 19A shows how chord diagrams generated upon CellChat utilization representing Spp1 pathway signaling directions within the scRNA-seq datasets populations. FIG. 19B shows scRNA-seq data analysis using CellChat approach for outgoing signaling pattern predicted top pathways in NVAMs (indicated in arrows). The number and level of expressed proteins from NVAMs were increased in retina with OIR and s-OIR, particularly in macrophage 2 which was only very few under normal condition, such as top upregulated pathways in OIR: Spp1. MIF (macrophage Migration Inhibitory Factor). IGF (Insulin-like Growth Factor). BAFF (B cell Activating Factor). PD-L1 (Programmed Death Ligand 1), and MPZ (Myelin Protein Zero), and additional upregulated pathways in s-OIR: MHC-I (Major Histocompatibility Complex I). FN1 (Fibronectin 1). OSM (Oncostatin M), CD68, CXCL. CD137. VEGF, and PVR/CD155. s-OIR: myeloid SOCS3 deficient mice with OIR.

FIGS. 20A-20G show the phenotype of Spp1 knockout mice. FIGS. 20A-20B show how Spp1 deficiency (Spp1^{KO}) delayed the normal retinal vasculature development at P5 compared to littermate wild type mice (Spp1^{WT}), but not to heterogynous mice (Spp1^{Het}). (n=7-14 retinas). FIGS. 20C-20D show how Spp1 deficiency (Spp1^{KO}) did not influence VO at P12 in OIR mice compared to littermate Spp1^{WT} and Spp1^{Het} OIR mice (n=8-9 retinas). FIGS. 20E-20G show how Spp1 deficiency (Spp1^{KO}) did not influence NV at P17 in OIR mice compared to littermate Spp1WT and Spp1^{Het} OIR mice, but increased VO compared to littermate Spp1^{WT} btu not Spp1^{Het} OIR mice at P17 (n=6-18 retinas). Scale bar in A, C, and E: 1000 μm. *, p<0.05; n.s., no significant.

### DETAILED DESCRIPTION OF CERTAIN EMBODIMENTS

Aspects of the present disclosure are based on the discovery of factors involved in the activation of myeloid lineage cells that contribute to retinal neovascularization and vision loss during retinopathies. Without wishing to be bound by theory, retinopathies represent a set of distinct but similar diseases in which damage to the retina is characterized by an inflammatory response, during which activated myeloid lineage cells infiltrate the retina. These myeloid lineage cells, which include microglial cells, macrophages, B cells, T cells, monocytes, and granulocytes, secrete a variety of cytokines and chemokines that promote the formation of new blood vessels (neovascularization) in the retina. The formation of these blood vessels in the retina is a major contributing factor for vision loss. Although retinopathies can be treated to slow the progression of vision loss, they generally cannot be cured. Several distinct types of retinopathy have been recognized to date, which may occur at any point during an individual's lifespan and may affect different parts of the retina.

Retinopathy of prematurity (ROP) is a type of retinopathy that affects approximately 16,000 preterm infants in the United States each year (contributing to between 6-18% of blindness in the U.S.) and can cause lifelong vision impairment. ROP is characterized by in initial loss of blood vessels in the retinal, followed by over proliferation of blood vessels. Over proliferation of blood vessels can cause scarring of the retina, which can result in retinal detachment and blindness. Although ROP can be treated using laser therapy, cryotherapy, and/or treatment with inhibitors of vascular endothelial growth factor (VEGF), these treatments frequently have undesirable side effects. For example, while laser therapy can restore vision at the center of this visual field, this may occur at the expense of peripheral vision. Additionally, both laser therapy and cryotherapy typically require general anesthesia, which is undesirable for treatment of preterm infants.

Unlike ROP, diabetic retinopathy (DR) is a variety of retinopathy that occurs in patients with diabetes and is the most common cause of vision loss in adults. However, similar to ROP, DR also occurs in two phases. During early DR, also referred to as non-proliferative DR, new blood vessels do not form, but existing blood vessels weaken and leak blood into the retina. As a result, fluid may accumulate in the macula at the center of the retina, referred to as macular edema. During advanced DR, previously damaged blood vessels become closed off, instigating the growth of new blood vessels. As in ROP, scarring of these new blood vessels can lead to retinal detachment and blindness. DR can be treated by laser therapy, injection of VEGF inhibitor into the eye, and vitrectomy to remove accumulated blood from the vitreous fluid, however each of these therapies only slow the progression of DR and cannot cure it.

Like DR, another common retinopathy is age-related macular degeneration (AMD), which is most common in adults over 50 years old. AMD is a degenerative condition that mainly affects the macula during advanced age. AMD can occur in a “dry” form and a “wet” form. During dry AMD, photoreceptors in the macula degenerate, while fatty deposits called drusen accumulate under the retina. Dry AMD progresses slowly and is associate with a gradual loss of central vision, distortions in vision, increased difficulty with vision in low light, or a decreased ability to differentiate between colors. Dry AMD can progress to wet AMD, during which blood vessels proliferate and may hemorrhage. Wet AMD is typically associated with a sudden or rapid loss of visual acuity. Like other retinopathies, dry AMD and wet AMD can be treated with surgery but are incurable.

Retinopathies can also occur due to genetic factors. Retinitis pigmentosa (RP) is a collection of inherited retinopathies which can be caused by inherited mutations in over 100 genes involved in the formation and maintenance of photoreceptor cells. RP can be inherited in cither an autosomal dominant or autosomal recessive manner, although approximately 25% of RP diagnoses are found to occur due to an autosomal dominant form. Other forms of RP are inherited in an X chromosome-linked or a Y chromosome-linked inheritance pattern. Common forms of RP occur due to mutations in retinal pathway genes and include mutations in the gene encoding rhodopsin, a G-protein coupled receptor in rod cells of the retina that is required for phototransduction. During RP. photoreceptor rod cells progressively die or otherwise become incapable of phototransduction, causing progressive loss of vision, particularly peripheral vision.

Over time, photoreceptor cone cells begin to die as well and can lead to tunnel vision, where only vision at the center of the visual field remains. Complete blindness is rare in RP. During the progression of RP blood vessels may become narrower, reducing blood flow to the retina, which can stimulate the formation of new blood vessels. Although the progression of RP can be treated with vitamin A intake, similar to other forms of retinopathy it cannot be cured.

Given that various types of retinopathy can be treated, but not cured, and considering that these treatments can have undesirable side effects (e.g., loss of peripheral vision as a result of laser surgery), the need for improved therapies remains high. One strategy for therapy is to directly address the underlying cause of neovascularization, rather than attempting to rectify the symptoms (e.g., vision loss) that results from this process. Potential therapies may therefore modulate the activity of the myeloid lineage immune cells that drive neovascularization, in order to prevent this activity. To date, the involvement of some factors during myeloid cell activation has been recognized. In particular, suppressor of cytokine signaling 3 (SOCS3) has been shown to have a protective effect on the retina by downregulating the release of proinflammatory cytokines and chemokines that contribute to inflammation during retinopathy (see, e.g., Sun Y, et al. “SOCS3 in retinal neurons and glial cells suppresses VEGF signaling to prevent pathological neovascular growth.” Sci Signal. 2015; 8 (395): ra94; and Wang T, et al. “Myeloid lineage contributes to pathological choroidal neovascularization formation via SOCS3.” EBioMedicine. 2021; 73:103632, the entire contents of which are incorporated by reference herein), while cellular Fos proto-oncogene (c-Fos) and vascular endothelial growth factor A (VEGFA) have been shown to promote neovascularization (see, e.g., Sun Y, et al. “Inflammatory signals from photoreceptor modulate pathological retinal angiogenesis via c-Fos.” J Exp Med. 2017; 214 (6): 1753-1767, the entire contents of which are incorporated by reference herein). However, it is incompletely understood which other factors are involved in the activation of myeloid lineage cells during neovascularization. Described herein are additional factors that are recognized to promote the activation of myeloid lineage cells, as well as methods for reducing the activation of myeloid lineage cells by reducing the expression and or activity of such factors.

### Administration of Agents to Decrease the Activity of Factors Involved in Myeloid Cell Activation

The present disclosure provides for methods of reducing the activation of myeloid lineage cells in a subject. In some embodiments, the activation of myeloid lineage cells in a subject is reduced by administering to the subject or myeloid lineage cells of the subject an effective amount of one or more agents that is sufficient to result in a decrease in the expression and/or activity of a factor associated with myeloid cell activation. In some embodiments, a factor associated with myeloid cell activation is any factor disclosed herein that is typically upregulated in myeloid lineage cells during myeloid cell activation and/or during a retinal disease, such as a retinopathy, a retinal injury, or a glaucoma that is associated with a retinopathy. In some embodiments, the methods disclosed herein are useful for the treatment of retinal disease, such as a retinopathy, a retinal injury, or a glaucoma that is associated with a retinopathy.

In some aspects, the present disclosure provides a method for reducing activation of myeloid lineage cells in a subject, the method comprising administering to a subject in need thereof an effective amount of an agent that results in a decrease in the expression and/or activity of a factor associated with myeloid cell activation.

As used herein, the terms “administer,” “administering.” or “administration” refer to implanting, absorbing, ingesting, injecting, inhaling, or otherwise introducing an agent described herein, or a composition thereof (e.g., a pharmaceutical composition), in or on a subject. As used herein, the term “treatment,” “treat,” and “treating” refers to the application or administration of an agent described herein, or a composition thereof (e.g., a pharmaceutical composition), to a subject in need thereof for the purpose of reducing the severity of a disease (e.g., a retinopathy) in the subject. A “subject in need thereof” refers to an individual that has a disease, a symptom of the disease, or a predisposition toward the disease. A method for treating a disease may encompass administering to a subject an agent described herein, or a composition thereof (e.g., a pharmaceutical composition) with the intention to cure, heal, alleviate, relieve, alter, remedy, ameliorate, improve, or affect the disease, a symptom of the disease, or predisposition toward the disease in the subject. A method for treating a disease may encompass prophylaxis, wherein an agent is administered to the subject for the purpose of preventing development of the disease, for example, in a subject that is not known to have the disease, but may develop or be at risk of developing the disease in the future.

As used herein, a “therapeutically effective amount” or “effective amount” refers to the amount of an agent that is sufficient to elicit the desired biological response in the subject, for example, alleviating one or more symptoms of the disease (e.g., a retinopathy). A therapeutically effective amount may be an amount that is either administered to the subject alone or in combination with one or more other agents. Effective amounts vary, as recognized by those skilled in the art, depending on such factors as the desired biological endpoint, the pharmacokinetics of the administered agent, the particular condition or disease being treated, the severity of the condition or disease, the individual parameters of the subject, including age, physical condition, size, gender and weight, the duration of the treatment, the nature of any other concurrent therapy, the specific route of administration, and like factors that are within the knowledge and expertise of the health practitioner to determine. These factors are well known to those of ordinary skill in the art and can be addressed with no more than routine experimentation. It is generally preferred that a maximum dose of the individual agents described herein or any combinations thereof to be used is at most the highest dose that can be safely administered to the subject according to sound medical judgment. Preferably, an effective dose is lower than the highest dose that can be safely administered to the subject. It will be understood by those of ordinary skill in the art, however, that a subject or health practitioner may select a lower dose (e.g., the minimum effective dose) in order to mitigate any potential risks of treatment, such as side effects of the treatment.

In some embodiments, for an adult subject of normal weight, doses ranging from about 0.01 to 1000 mg/kg of an agent may be administered. In some embodiments, the dose is between 1 to 200 mg. The particular dosage regimen, i.e., the dose, timing, and repetition, will depend on the particular subject and that subject's medical history, as well as the properties of the agent (such as the pharmacokinetics of the agent) and other consideration well known in the art.

Treating a disease (e.g., a retinopathy) may include delaying the development or progression of the disease or reducing disease severity. Treating the disease does not necessarily require curative results. As used herein, “delaying” the development of a disease means to defer, hinder, slow, retard, stabilize, and/or postpone progression of the disease in a subject. Delaying the progression of a disease may include delaying or preventing the spread of a disease occurring in a subject. This delay can be of varying lengths of time, depending on the history of the disease and/or individuals being treated. A method that delays the development of a disease, or delays the onset of the disease, is a method that reduces probability of developing one or more symptoms of the disease in a given time frame and/or reduces extent of the symptoms in a given time frame, as compared to the absence of such a method. Comparisons are typically based on clinical studies, using a number of subjects sufficient to give a statistically significant result.

The “development” or “progression” of a disease (e.g., a retinopathy) refers to initial manifestations and/or ensuing progression of the disease in a subject. Development of a disease can be detectable and assessed using standard clinical techniques as well known in the art. However, development also refers to progression that may be undetectable. For purpose of this disclosure, development or progression may refer to the development or progression of symptoms of a disease (e.g., a retinopathy). The term “development” includes the occurrence, recurrence, and onset of a disease. As used herein “onset” or “occurrence” of a disease includes the initial onset of a disease, as well as recurrence of the disease (i.e., in a subject who has had the disease previously).

A “subject” to which administration is contemplated refers to a human (i.e., male or female of any age group, e.g., pediatric subject (e.g., infant, child, or adolescent) or adult subject (e.g., young adult, middle-aged adult, or senior adult)) or a non-human animal. In some embodiments, the non-human animal is a mammal (e.g., rodent, e.g., mouse or rat), a primate (e.g., cynomolgus monkey or rhesus monkey), a commercially relevant mammal (e.g., cattle, pig, horse, sheep, goat, cat, or dog), or a bird (e.g., commercially relevant bird, such as chicken, duck, goose, or turkey). The non-human animal may be a male or female at any stage of development and may be a juvenile animal or an adult animal. The non-human animal may be a transgenic animal or genetically engineered animal.

In some embodiments, the subject is a companion animal (e.g., a pet or service animal). “A companion animal,” as used herein, refers to a pet and other domestic animal. Non-limiting examples of companion animals include dogs and cats; livestock such as horses, cattle, pigs, sheep, goats, and chickens; and other animals such as mice, rats, guinea pigs, and hamsters. In some embodiments, the subject is a research animal. Non-limiting examples of research animals include rodents (e.g., rats, mice, guinea pigs, and hamsters), rabbits, or non-human primates.

In some embodiments, the agent to be administered to a subject is an agent that results in a decrease in the expression and/or activity of a factor associated with myeloid cell activation. As used herein, the term “expression” refers to the expression of gene encoding a factor associated with myeloid cell activation in a cell to produce a mRNA molecule which is translated by one or more ribosomes to produce the factor. As used herein, the term “activity” refers to one or more activities of the factor associated with myeloid cell activation, and may include, but is not limited to, one or more enzymatic activities of the factor or one or more interactions between the factor and another molecule, such as a protein, a peptide, a nucleic acid, a lipid, a carbohydrate, or an ion. An agent described herein may reduce the expression or activity of a factor associated with myeloid cell activation, and may reduce both the expression and activity of the factor. Those of ordinary skill in the art will readily recognize that an agent that reduces the expression of a given factor will generally also reduce the activity of the factor by reducing the total number of molecules of the factor that are produced by a cell. An agent described herein may permanently (irreversibly) reduce the expression and/or activity of a factor associated with myeloid cell activation, or may transiently (reversibly) reduce the expression and/or activity of the factor.

In some embodiments, the agent to be administered to a subject is an agent that reduces the expression and/or activity of a factor associated with myeloid cell activation that is disclosed herein. In some embodiments, the agent to be administered to a subject is an agent that reduces the expression and/or activity of a factor associated with myeloid cell activation that is upregulated in myeloid lineage cells during retinopathy. In some embodiments, the agent to be administered to a subject is an agent that reduces the expression and/or activity of a single factor associated with myeloid cell activation. In some embodiments, the agent to be administered to a subject is an agent that reduces the expression and/or activity of more than one factor associated with myeloid cell activation. In some embodiments, more than one agent is administered to a subject to reduce the expression and/or activity of one or more factors associated with myeloid cell activation (i.e., 1 factor associated with myeloid cell activation, 2 factors associated with myeloid cell activation, 3 factors associated with myeloid cell activation, or more).

In some embodiments, the agent is an agent that reduces the expression and/or activity of secreted phosphoprotein 1 (Spp1), also referred to as osteopontin (OPN), an extracellular structural protein that is secreted by a variety of immune cells and is involved with the cytokine release and chemotaxis of certain immune cells (e.g., neutrophils, activated T helper 1 (Th1) cells) during inflammatory responses. In some embodiments, the agent reduces the expression and/or activity of human Spp1 (e.g., NCBI Reference Sequence: NP_110418.1; Gene ID: 6696).

In some embodiments, the agent is an agent that reduces the expression and/or activity of triggering receptor expressed on myeloid cells 2 (Trem2), a receptor protein on myeloid cells the interacts with DNAX activator proteins 10 and 12 (DAP10, DAP12) to modulate the expression of genes involved in inflammatory responses. In some embodiments, the agent reduces the expression and/or activity of human Trem2 (e.g., NCBI Reference Sequence: NP_061838.1; Gene ID: 54209).

In some embodiments, the agent is an agent that reduces the expression and/or activity of translocator protein (Tspo), also referred to as peripheral benzodiazepine receptor (PBR), an 18 kDa protein on the outer mitochondrial membrane that is involved with oxidative bursts and cytokine release in neutrophils and macrophages. In some embodiments, the agent reduces the expression and/or activity of human Tspo (e.g., NCBI Reference Sequence: NP_000705.2; Gene ID: 706).

In some embodiments, the agent is an agent that reduces the expression and/or activity of signal transducer and activator of transcription 3 (STAT3), a transcription factor that promotes in the expression of several genes involved in the mediation of inflammatory responses, including Spp1. In some embodiments, the agent reduces the expression and/or activity of human STAT3 (e.g., NCBI Reference Sequence: NP_644805.1; Gene ID: 6774).

In some embodiments, the agent is an agent that inhibits the expression of a factor associated with myeloid cell activation (e.g., Spp1, Trem2, Tspo, STAT3). In some embodiments, the agent comprises a small interfering RNA (siRNA), a short hairpin RNA (shRNA), or an antisense oligonucleotide (ASO) that is complementary to a gene encoding the factor associated with myeloid cell activation. In some embodiments, the agent comprises a viral vector that encodes a siRNA or a shRNA that is complementary to a gene encoding the factor associated with myeloid cell activation (e.g., Spp1, Trem2, Tspo, STAT3). Examples of viral vector known in the art that are suitable for delivery of siRNA or shRNA include, but are not limited to, lentiviral vectors and recombinant adeno-associated viral vectors (rAAV). In some embodiments, a siRNA, shRNA, or ASO that inhibits expression of a factor associated with myeloid cell activation (e.g., Spp1, Trem2, Tspo, STAT3) is at least 60%, at least 70%, at least 80%, at least 90%, at least 95%, at least 99%, or 100% complementary to a region of a gene encoding the factor associated with myeloid cell activation (e.g., Spp1, Trem2, Tspo, STAT3). In some embodiments, an agent that inhibits the expression of a factor associated with myeloid cell activation (e.g., Spp1, Trem2. Tspo, STAT3) further reduces the overall activity of the factor associated with myeloid cell activation (e.g., Spp1, Trem2, Tspo, STAT3).

In some embodiments, the agent is an agent that inhibits the activity of the factor associated with myeloid cell activation (e.g., Spp1, Trem2, Tspo, STAT3). In some embodiments, the agent is a protein, a peptide, an aptamer, or a small molecule that inhibits the activity of the factor associated with myeloid cell activation (e.g., Spp1, Trem2, Tspo, STAT3), the meaning of each of which is well known to those of ordinary skill in the art. In some embodiments, the agent binds to (physically interacts with) the factor associated with myeloid cell activation (e.g., Spp1, Trem2, Tspo, STAT3). In some embodiments, the agent that inhibits the activity of the factor associated with myeloid cell activation (e.g., Spp1, Trem2, Tspo, STAT3) is an antibody. In some embodiments, the agent is an antibody that binds specifically to the factor associated with myeloid cell activation (e.g., Spp1, Trem2, Tspo, STAT3). In some embodiments, the agent is an antibody that binds preferentially to the factor associated with myeloid cell activation (e.g., Spp1, Trem2, Tspo, STAT3), and may bind to one or more other species.

In some embodiments, a subject is further administered a second agent. In some embodiments, the second agent is an agent that results in an increase in the expression and/or activity of suppressor of cytokine signaling 3 (SOCS3). Without wishing to be bound by theory, a second agent that results in an increase in the expression and/or activity of SOCS3 enhances a protective effect of SOCS3 in the retina of the subject. In some embodiments, the second agent is an agent that results in a decrease in the expression and/or activity of cellular Fos proto-oncogene (c-Fos) or vascular endothelial growth factor A (VEGFA). Without wishing to be bound by theory, a second agent that results in a decrease in the expression and/or activity of c-Fos or VEGFA suppresses a harmful effect of c-Fos or VEGFA in the retina of the subject. A second agent may be any type of agent contemplated herein or otherwise known in the art, such as, but not limited to, a RNA, a protein, a peptide, an aptamer, or a small molecule, the meaning of each of which is well known to those of ordinary skill in the art.

Conventional methods, known to those of ordinary skill in the art of medicine, can be used to administer the agent (or agents) to the subject, depending upon the type of disease to be treated (e.g., a retinopathy) or the site of the disease (e.g., the retina). The agent (or agents) can be administered systemically (i.e., throughout the body) or locally (i.e., to one or more specific organs, tissues, or locations in the body). The agent (or agents) can also be administered via any conventional route, e.g., administered orally, parenterally, by inhalation spray, topically, rectally, nasally, buccally, vaginally, intraperitoneally, or via an implanted reservoir. The term “parenteral” as used herein includes subcutaneous, intracutaneous, intravenous, intramuscular, intraarticular, intraarterial, intrasynovial, intrasternal, intraperitoneal, intrathecal, intralesional, and intracranial injection or infusion techniques. In some embodiments, an agent described herein is administered via intravenous injection or infusion. In some embodiments, an agent described herein is administered intraocularly, for example, by an intraocular injection. In addition, an agent described herein may be administered to the subject via injectable depot routes of administration such as using 1-, 3-, or 6-month depot injectable or biodegradable materials and methods. In embodiments where more than one agent is administered to a subject, the agents may be administered by the same delivery route or by different delivery route. In embodiments where more than one agent is administered to a subject, the agents may be administered simultaneously (e.g., at the same time as part of the same administered composition (e.g., pharmaceutical composition), or as separate compositions (e.g., pharmaceutical compositions)). In embodiments where more than one agent is administered to a subject, the agents may be administered sequentially (e.g., at different times as separate compositions (e.g., pharmaceutical compositions)).

In some embodiments, an agent described herein is administered to the subject more than once. In some embodiments, an agent described herein is administered to the subject once per day, once per 2 days, once per 3 days, once per 4 days, once per 5 days, once per 6 days, once per week, once per 2 weeks, once per 3 weeks, once per month, once per 2 months, once per 3 months, once per 4 months, once per 6 months, once per 7 months, once per 8 months, once per 9 months, once per 10 months, once per 11 months, or once per year.

In some embodiments, the administration results in a decrease in the expression and/or activity of the factor associated with myeloid cell activation (e.g., Spp1, Trem2, Tspo, STAT3) by up to 10%, up to 20%, up to 30%, up to 40%, up to 50%, up to 60%, up to 70%, up to 80%, up to 90%, up to 95%, up to 99%, or up to 100%, as compared to expression and/or activity of the factor associated with myeloid cell activation (e.g., Spp1, Trem2, Tspo, STAT3) prior to the administration. In some embodiments, the expression and/or activity of the factor associated with myeloid cell activation (e.g., Spp1, Trem2, Tspo, STAT3) is decreased in myeloid lineage cells of the subject. In some embodiments, the administration results in reduced activation of myeloid lineage cells in the subject by up to 10%, up to 20%, up to 30%, up to 40%, up to 50%, up to 60%, up to 70%, up to 80%, up to 90%, up to 95%, up to 99%, or up to 100%, as compared to myeloid lineage cell activation in the absence of the administration. As used herein, the term “activation” in respect to myeloid lineage cells may refer to an increase in any activity of a myeloid lineage cell, such as, but not limited to, the proliferation of one or more types of myeloid lineage cells in a tissue or organ (e.g., an increase in the quantity of myeloid lineage cells present in a tissue or organ, e.g., the retina) or the release of proinflammatory cytokines and/or chemokines from one or more types of myeloid lineage cells in a tissue or organ. In some embodiments, the myeloid lineage cells are myeloid lineage cells occurring in the retina of the subject. In some embodiments, the myeloid lineage cells are microglial cells (microglia), macrophages, B cells, T cells, monocytes, or granulocytes, or a subtype of such a cell that is known in the art. In some embodiments, the myeloid lineage cells are myeloid lineage cells in which expression and/or activity of Spp1. Trem2, Tspo, and/or STAT3 is upregulated prior to administration. In some embodiments, the myeloid lineage cells are myeloid lineage cells in which expression and/or activity of SOCS3 is downregulated prior to administration.

In some embodiments, the administration results in reduced neovascularization in the retina of the subject by up to 10%, up to 20%, up to 30%, up to 40%, up to 50%, up to 60%, up to 70%, up to 80%, up to 90%, up to 95%, up to 99%, or up to 100%, as compared to neovascularization in the retina of the subject in the absence of the administration.

In some embodiments, the subject (e.g., a human patient) is a subject that has, is suspected of having, or is at risk of developing a retinopathy, a retinal injury, or a glaucoma associated with retinopathy. In some embodiments, the retinopathy is retinopathy of prematurity (ROP), dry age-related macular degeneration (AMD), wet AMD, retinal degeneration, retinitis pigmentosa, diabetic retinopathy, or another retinopathy known to those of ordinary skill in the art. In some embodiments, a retinal injury is a retinal tear, retinal detachment, an epiretinal membrane that is present on the retina, a macular tear, or a macular hole. In some embodiments, a glaucoma associated with retinopathy is any glaucoma (i.e., damage to the optic nerve, frequently occurring as a result of increased optic pressure) that occurs with a retinopathy described herein or otherwise known to those of ordinary skill in the art. In some embodiments, the administration treats the retinopathy, retinal injury, or glaucoma associated with retinopathy in the subject. In some embodiments, the administration prevents the development of the retinopathy, retinal injury, or glaucoma associated with retinopathy in the subject.

Cell Therapy for the Treatment of Retinal Disorders Associated with Neovascularization

In further aspects, the present disclosure provides a method for treating a retinopathy, a retinal injury, or a glaucoma associated with retinopathy in a subject, the method comprising collecting myeloid lineage cells from the subject in need thereof, treating the myeloid lineage cells with an agent that results in a decrease in the expression and/or activity of a factor associated with myeloid cell activation (e.g., Spp1, Trem2, Tspo, STAT3), and administering the myeloid lineage cells to the subject, thereby treating a retinopathy, a retinal injury, or a glaucoma associated with retinopathy in the subject.

In some embodiments, the agent that results in a decrease in the expression and/or activity of a factor associated with myeloid cell activation is an agent that results in the expression and/or activity of Spp1, Trem2, Tspo, STAT3, or a combination thereof.

In some embodiments, the myeloid lineage cells are treated with is an agent that inhibits the expression of a factor associated with myeloid cell activation (e.g., Spp1, Trem2, Tspo, STAT3). In some embodiments, the agent comprises a small interfering RNA (siRNA), a short hairpin RNA (shRNA), or an antisense oligonucleotide (ASO) that is complementary to a gene encoding the factor associated with myeloid cell activation. In some embodiments, the agent comprises a viral vector that encodes a siRNA or a shRNA that is complementary to a gene encoding the factor associated with myeloid cell activation (e.g., Spp1, Trem2, Tspo, STAT3). Examples of viral vector known in the art that are suitable for delivery of siRNA or shRNA include, but are not limited to, lentiviral vectors and recombinant adeno-associated viral vectors (rAAV). In some embodiments, a siRNA, shRNA, or ASO that inhibits expression of a factor associated with myeloid cell activation (e.g., Spp1, Trem2. Tspo, STAT3) is at least 60%, at least 70%, at least 80%, at least 90%, at least 95%, at least 99%, or 100% complementary to a region of a gene encoding the factor associated with myeloid cell activation (e.g., Spp1, Trem2, Tspo, STAT3). In some embodiments, an agent that inhibits the expression of a factor associated with myeloid cell activation (e.g., Spp1. Trem2, Tspo, STAT3) further reduces the overall activity of the factor associated with myeloid cell activation (e.g., Spp1. Trem2, Tspo, STAT3).

In some embodiments, the myeloid lineage cells are treated with an agent that inhibits the activity of the factor associated with myeloid cell activation (e.g., Spp1, Trem2, Tspo, STAT3). In some embodiments, the agent is a protein, a peptide, an aptamer, or a small molecule that inhibits the activity of the factor associated with myeloid cell activation (e.g., Spp1, Trem2, Tspo, STAT3), the meaning of each of which is well known to those of ordinary skill in the art. In some embodiments, the agent binds to (physically interacts with) the factor associated with myeloid cell activation (e.g., Spp1, Trem2, Tspo, STAT3). In some embodiments, the agent that inhibits the activity of the factor associated with myeloid cell activation (e.g., Spp1, Trem2, Tspo, STAT3) is an antibody. In some embodiments, the agent is an antibody that binds specifically to the factor associated with myeloid cell activation (e.g., Spp1, Trem2, Tspo, STAT3). In some embodiments, the agent is an antibody that binds preferentially to the factor associated with myeloid cell activation (e.g., Spp1, Trem2, Tspo, STAT3), and may bind to one or more other species.

In some embodiments, the myeloid lineage cells are further treated with a second agent. In some embodiments, the second agent is an agent that results in an increase in the expression and/or activity of suppressor of cytokine signaling 3 (SOCS3). In some embodiments, the second agent is an agent that results in a decrease in the expression and/or activity of cellular Fos proto-oncogene (c-Fos) or vascular endothelial growth factor A (VEGFA). A second agent may be any type of agent contemplated herein or otherwise known in the art, such as, but not limited to, a RNA, a protein, a peptide, an aptamer, or a small molecule, the meaning of each of which is well known to those of ordinary skill in the art.

In some embodiments, the treatment of the myeloid lineage cells results in a decrease in the expression and/or activity of the factor associated with myeloid cell activation (e.g., Spp1. Trem2. Tspo, STAT3) in the myeloid lineage cells by up to 10%, up to 20%, up to 30%, up to 40%, up to 50%, up to 60%, up to 70%, up to 80%, up to 90%, up to 95%, up to 99%, or up to 100%, as compared to expression and/or activity of the factor associated with myeloid cell activation (e.g., Spp1, Trem2, Tspo, STAT3) prior to the treatment. In some embodiments, the treatment of the myeloid lineage cells results in reduced activation of the myeloid lineage cells by up to 10%, up to 20%, up to 30%, up to 40%, up to 50%, up to 60%, up to 70%, up to 80%, up to 90%, up to 95%, up to 99%, or up to 100%, as compared activation of the myeloid lineage cells prior to the treatment. In some embodiments, the activation of the myeloid lineage cells comprises proliferation of the myeloid lineage cells. In some embodiments, the activation of the myeloid lineage cells comprises the release of one or more proinflammatory cytokines or chemokines from the myeloid lineage cells.

In some embodiments, the myeloid lineage cells are microglial cells (microglia), macrophages, B cells, T cells, monocytes, or granulocytes, or a subtype of such a cell that is known in the art. In some embodiments, the myeloid lineage cells are collected from bone marrow of the subject prior to treatment. In some embodiments, the myeloid lineage cells are administered to the retina of the subject after treatment.

In some embodiments, the administration of the myeloid lineage cells to the subject results in reduced neovascularization in the retina of the subject by up to 10%, up to 20%, up to 30%, up to 40%, up to 50%, up to 60%, up to 70%, up to 80%, up to 90%, up to 95%, up to 99%, or up to 100%, as compared to neovascularization in the retina of the subject in the absence of the administration.

In some embodiments, the subject is a mammalian subject (e.g., a domestic animal, a pet, a research animal). In some embodiments, the subject is a human.

In some embodiments, the retinopathy to be treated in the subject is retinopathy of prematurity (ROP), dry age-related macular degeneration (AMD), wet AMD, retinal degeneration, retinitis pigmentosa, diabetic retinopathy, or another retinopathy known to those of ordinary skill in the art. In some embodiments, a retinal injury is a retinal tear, retinal detachment, an epiretinal membrane that is present on the retina, a macular tear, or a macular hole. In some embodiments, a glaucoma associated with retinopathy is any glaucoma that occurs with a retinopathy described herein or otherwise known to those of ordinary skill in the art.

Those of skill in the art will recognize that the methods of treatment described herein may also be suitable for the treatment of other diseases or disorders associated with activation of myeloid lineage cells and neovascularization of the retina or other parts of the eye, particularly if such diseases or disorders cause progressive vision loss and/or eventual blindness.

### EXAMPLES

### Example 1: Myeloid SOCS3 Controls Immune-Vascular Interaction in Retinopathy Introduction

Ischemic retinopathies, such as retinopathy of prematurity (ROP), diabetic retinopathy, and age-related macular degeneration, are major causes of visual impairment and blindness (Blencowe et al., 2012; Hartnett and Penn, 2013; Lutty et al., 2006; Tasman et al., 2006). With advances in neonatal care, smaller and more premature infants can be saved, however, these same infants are at a high risk for ROP. Therefore, the incidence of ROP has continued to increase (Network et al., 2010). Inflammation and changes in immune function are involved in ROP (Chen et al., 2011; Dammann, 2010; Dammann et al., 2009; Hong et al., 2014; Lec and Dammann, 2012; Tolsma et al., 2011), but the association has not been well defined and the way to control inflammation is not clear. Current anti-inflammatory therapies, such as corticosteroids, have little detectable beneficial effect on ROP (Melamed et al., 2015). Non-steroidal anti-inflammatory drugs (NSAIDs) (Aranda et al., 2017) are commonly used in newborns for pharmacologic closure of the patent ductus arteriosus (Liebowitz and Clyman, 2017), but whether NSAIDs prevent ROP is unclear (Irmesi et al., 2014). However, immune cells including microglia can modulate retinal inflammation to regulate pathological retinal neovascularization (NV) (Chambers et al., 2013; Grunin et al., 2014; Kambhampati et al., 2015). Hypoxia attracts macrophages into hypoxic areas (Mitamura et al., 2005; Tsutsumi et al., 2003), where the hypoxia-activated macrophages and microglia proinflammatory and angiogenic cytokines. Both invading macrophages and resident microglia have been implicated in the pathogenesis of the retinopathy (Abcouwer, 2011). In a mouse model of ROP, oxygen-induced retinopathy (OIR) causes both recruited of blood-derived macrophages as well as microglia, which are shown to participate in the regulation of angiogenesis (Checchin et al., 2006; Gao et al., 2016; Kataoka et al., 2011; Zhou et al., 2015).

Neonates have immature immune systems and premature neonates with low birth weight are particularly susceptible to infection. It has been previously suggested that exposure of preterm newborns to infection and inflammatory mediators is associated with an increased risk for ROP (Chen et al., 2011; Tolsma et al., 2011). There have been higher rates of ROP in infants born to mothers having evidence of histologic chorioamnionitis compared with those born to mothers without signs of inflammation (Moscuzza et al., 2011; Polam et al., 2005). Infectious organisms and/or their microbial products may stimulate the production of proinflammatory cytokines, suggesting that increased systemic levels of cytokines are associated with an increased risk for ROP (Lee and Dammann, 2012). In a large cohort study, infants who developed early onset sepsis had an increased risk for severe ROP (Klinger et al., 2010). In the Extremely Low Gestational Age Newborns Study, the increased risk for ROP was primarily observed in association with late-onset neonatal sepsis (Tolsma et al., 2011). In the pre-phase of ROP, prenatal pro-inflammatory events contributed to fetal inflammation. Exposure to intrauterine infection and inflammation contributed to a “pre-phase” of ROP, sensitizing the pre-ROP retina for subsequent insults, thereby setting the stage for what are now referred to as phase I and phase II of ROP (Lee and Dammann, 2012).

Immune cells can produce cytokines and growth factors to endothelial cells and regulate retinal blood vessel growth, especially myeloid cells, which have been reported to play roles in the processes of retinal vascular normal development (Stefater et al., 2011a; Stefater et al., 2011b), vascular remodeling (Lang and Bishop, 1993), and vessel repair (Binet et al., 2013; Checchin et al., 2006; Davies et al., 2006; Ritter et al., 2006). Myeloid cells such as microglia, the primary resident immune cells of the retina, are rapidly activated after an inflammatory insult (Chen et al., 2002; Santos et al., 2008) and connect directly with blood vessels to modulate angiogenesis (Checchin et al., 2006; Stefater et al., 2011a). Understanding of pathological immune changes in retinopathies is currently limited due to lack of information about the physiology of immune cells in the healthy eye. Blood circulation in the eye contains a large number of immune cells of myeloid origin (Forrester et al., 2010; McMenamin, 1999; Yang et al., 1997), which has been immunohistochemically characterized in tissue flat-mounts and sections in mouse eyes (McMenamin, 1999). However, the role of immune-vascular crosstalk in ROP and in other retinopathies is largely unknown.

Suppressor of cytokine signaling 3 (SOCS3) is an immune regulator that controls innate and adaptive immunity, tissue inflammation, and cytokine production (Arnold et al., 2013; Qin et al., 2012; Spence et al., 2013). It was reported that SOCS3 could strongly suppress pathological ocular angiogenesis (Stahl et al., 2012; Sun et al., 2015a; Sun et al., 2015b). Deletion of SOCS3 in Tie2-expressing cells in the OIR model increased pathological NV via reduced feedback inhibition of the STAT3 and mTOR pathways, but not physiologic vascularization (Stahl et al., 2012). Neuronal and glial SOCS3 suppressed pathological retinal angiogenesis in the OIR mouse model by inhibiting the STAT3-mediated secretion of VEGF from these cells, which in turn suppressed endothelial cell activation. This resulted in decreased endothelial cell proliferation and angiogenesis. Suppression of SOCS3 attenuated the protective effects of retinoic-acid-receptor-related orphan receptor alpha (RORα) in retinopathy in the OIR mouse model. Inhibition of SOCS3, a direct transcriptional target of RORα, abolished the anti-inflammatory and vasoprotective effects of RORα deficiency. RORα suppression of SOCS3 promoted retinal inflammation, which reflected an endogenous inhibitory role of SOCS3 in blood vessels. Therefore, SOCS3 is an essential immune-regulator that mediates immune-vascular interaction in ocular neovascularization formation.

However, it remains unclear which other factors regulate neovascularization and are functionally connected to SOCS3. As described herein, SOCS3 was deleted or overexpressed in mouse immune cells of myeloid origin and the effects of this modulation were assessed. It was observed that myeloid specific deletion of SOCS3 significantly enhanced the number of myeloid cells present in the retina of mice with OIR and increased the expression of novel myeloid activation factors, while myeloid-specific overexpression of SOCS3 decreased pathological retinal neovascularization.

### Results

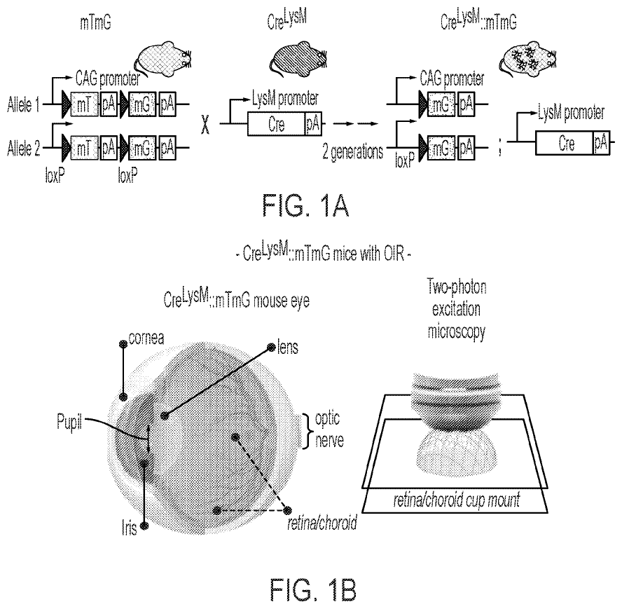

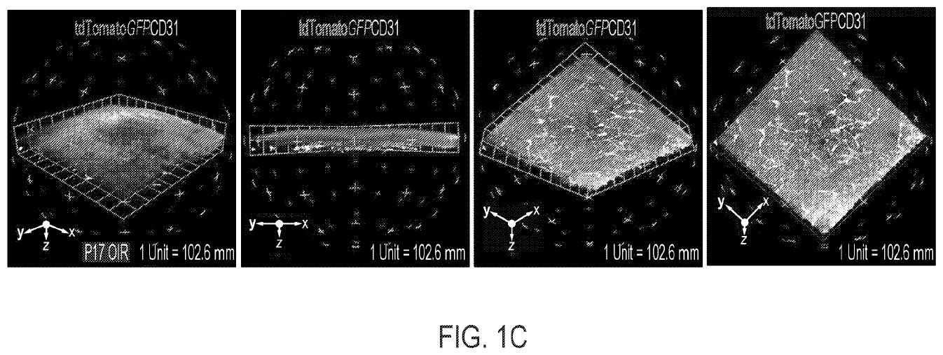

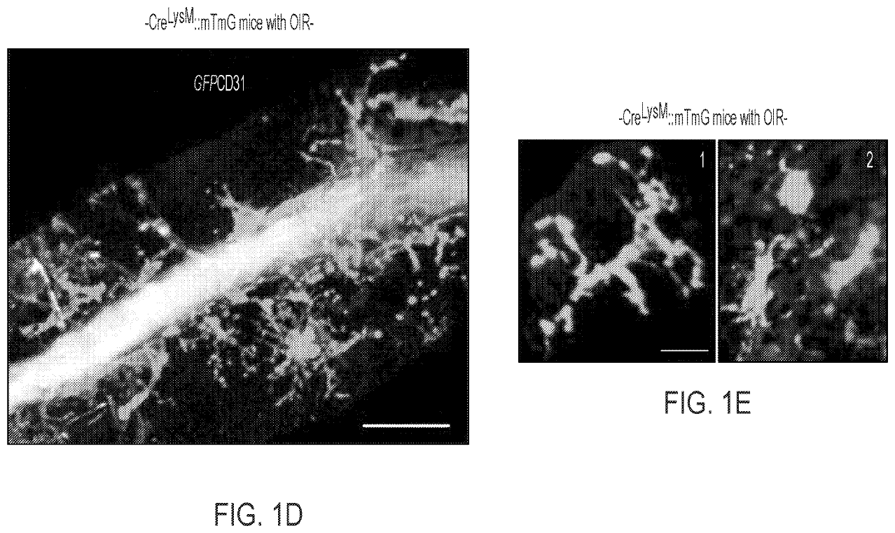

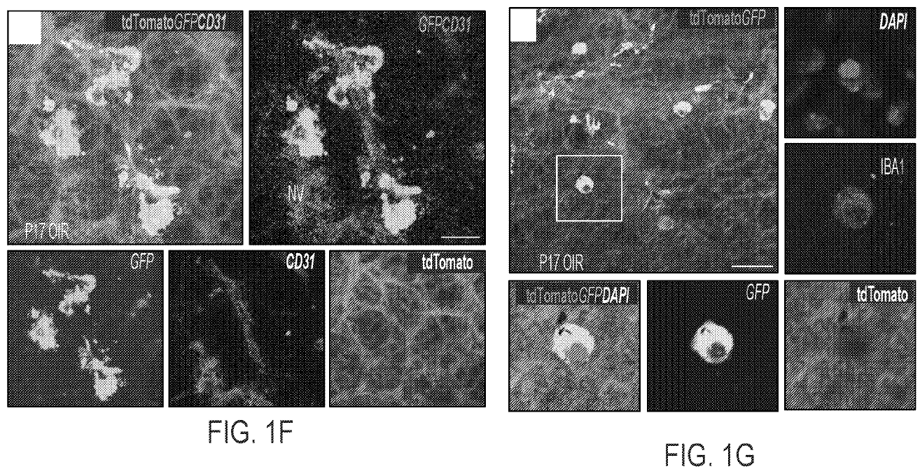

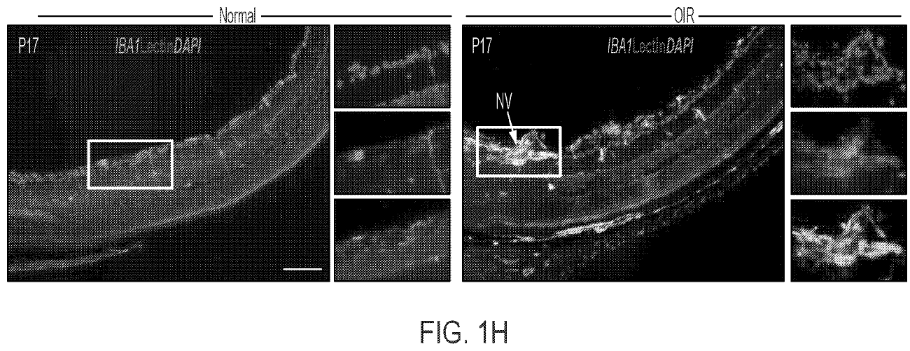

To study the effects of SOCS3 activity in a ROP mouse model, a reporter mouse model was developed in which all myeloid cells and all non-myeloid cells were differentially labeled with fluorescent labels (FIG. 1A). Retinas of sacrificed mice could then be excised and subsequently examined by a retina/choroid mount and two-photon excitation microscopy used to examine the presence of myeloid cells (FIGS. 1B-1E). Immunofluorescent staining further confirmed the presence of myeloid lineage cells in retinas, including microglia and macrophages (FIGS. 1F and 1G).

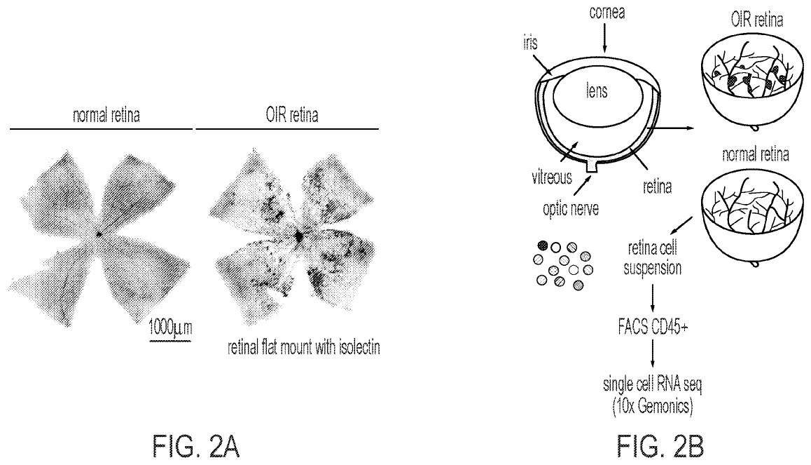

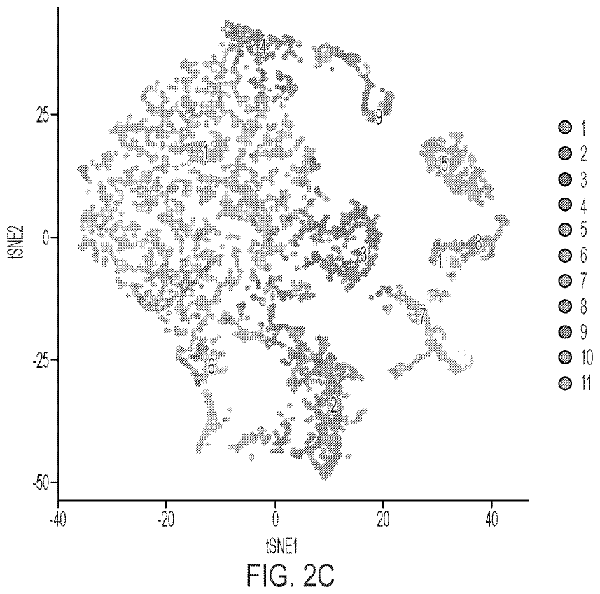

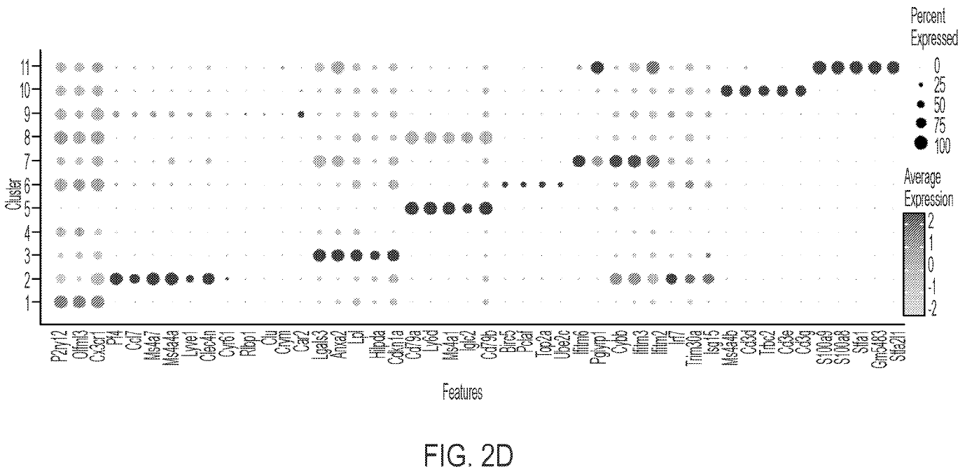

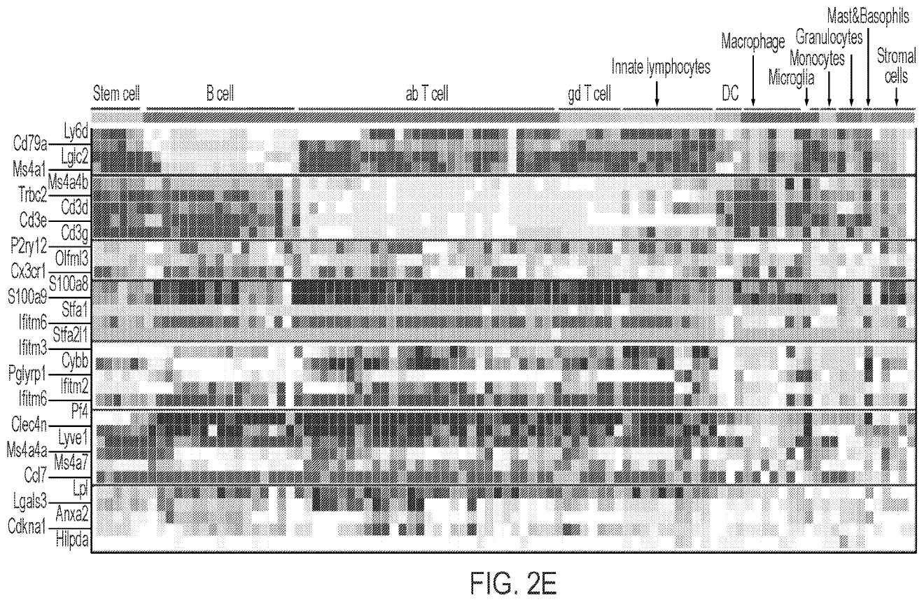

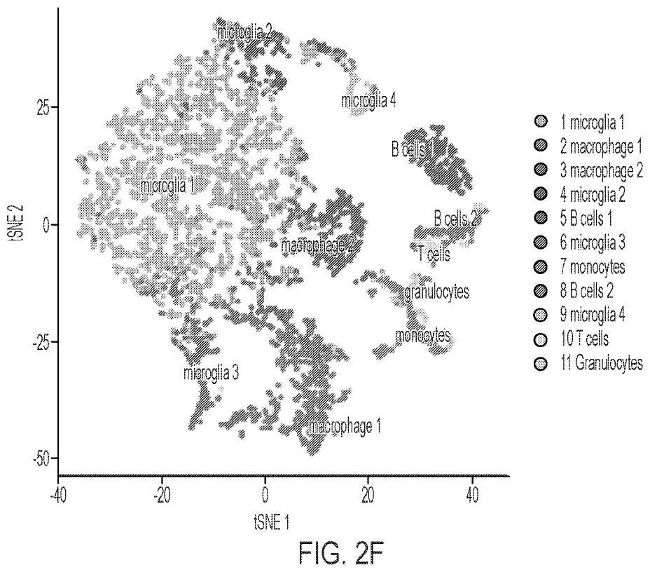

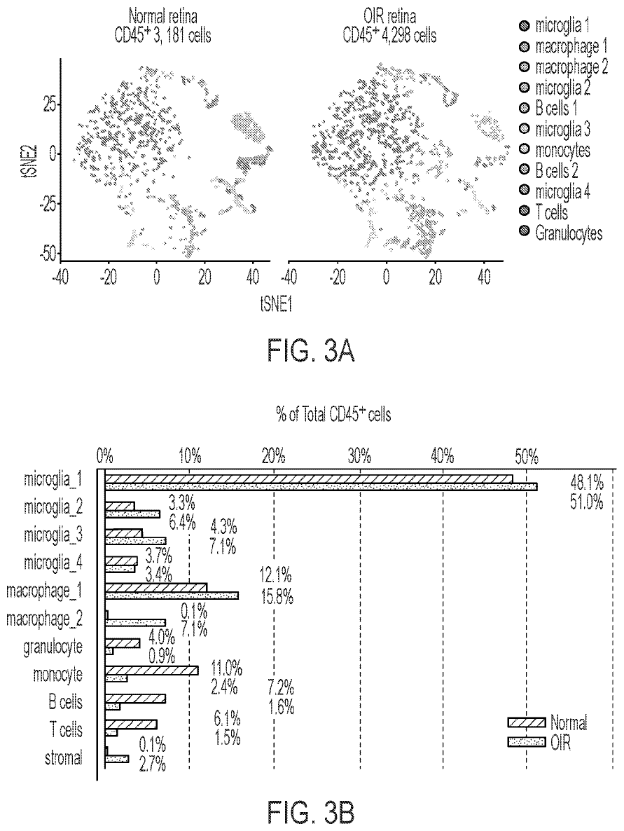

Compared to the retinas of normal, unaffected mice, the retinas of OIR mice displayed irregular blood vessel formation and organization (FIG. 2A). To determine how OIR changes the distribution and genetic expression of myeloid lineage cells in the retina, CD45+ myeloid cells were suspended from the retinas of healthy and OIR mice and subjected to single cell RNA sequencing (FIG. 2B). Myeloid cells in retinas of OIR mice were observed to sort into distinct cell type clusters, which could be identified based on the expression of myeloid biomarkers (FIGS. 2C-2F). It was observed that OIR caused an increase in the quantity of several myeloid cell types (FIGS. 3A and 3B).

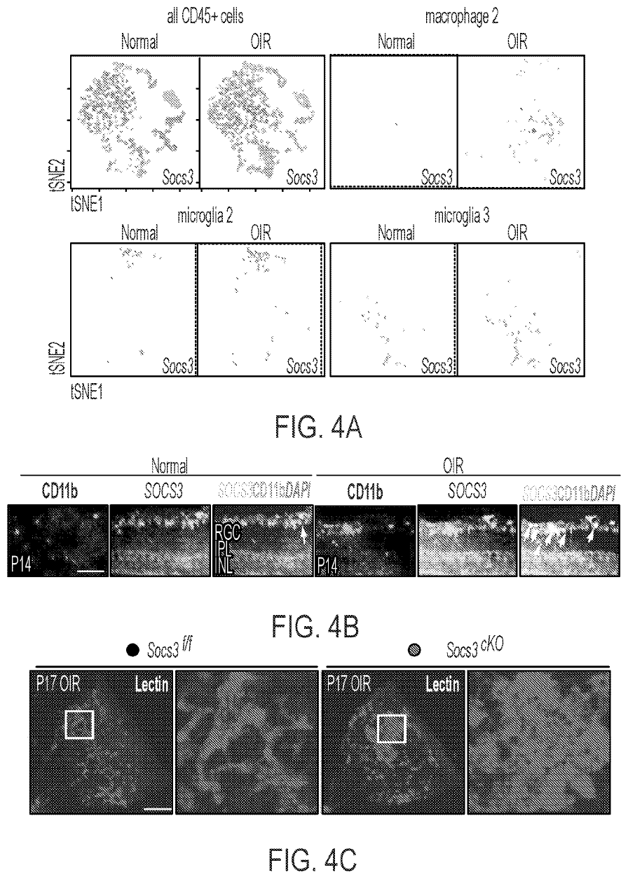

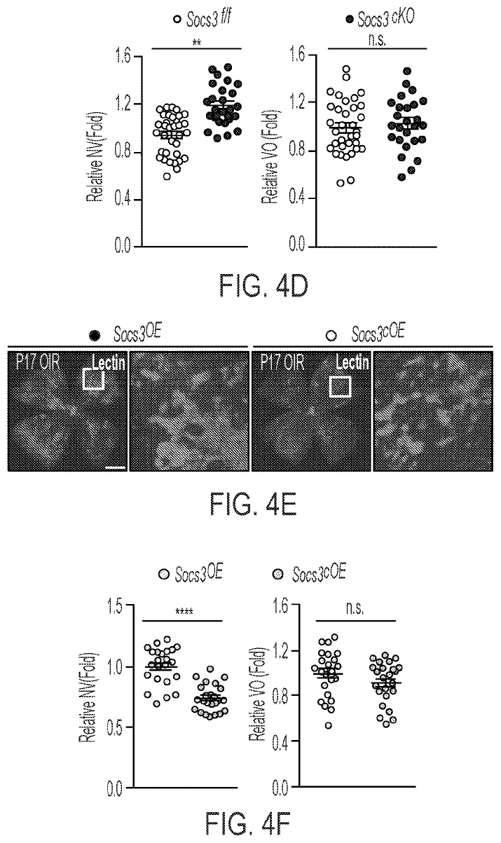

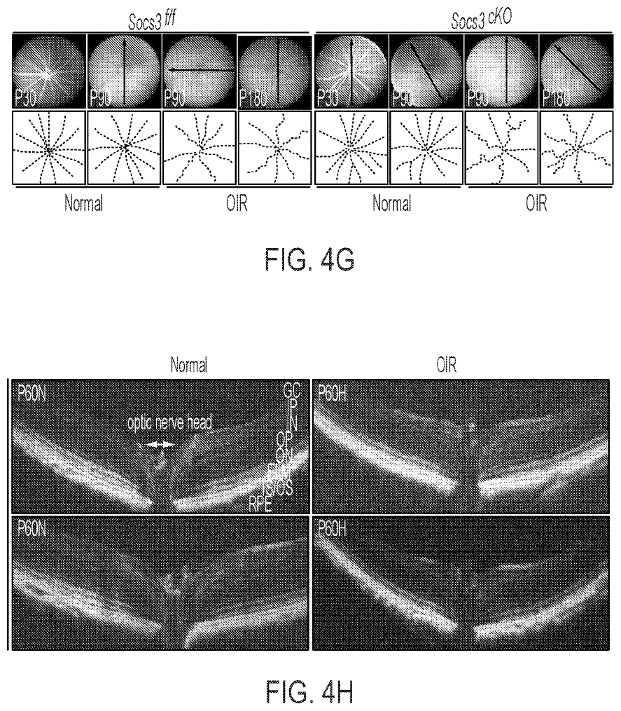

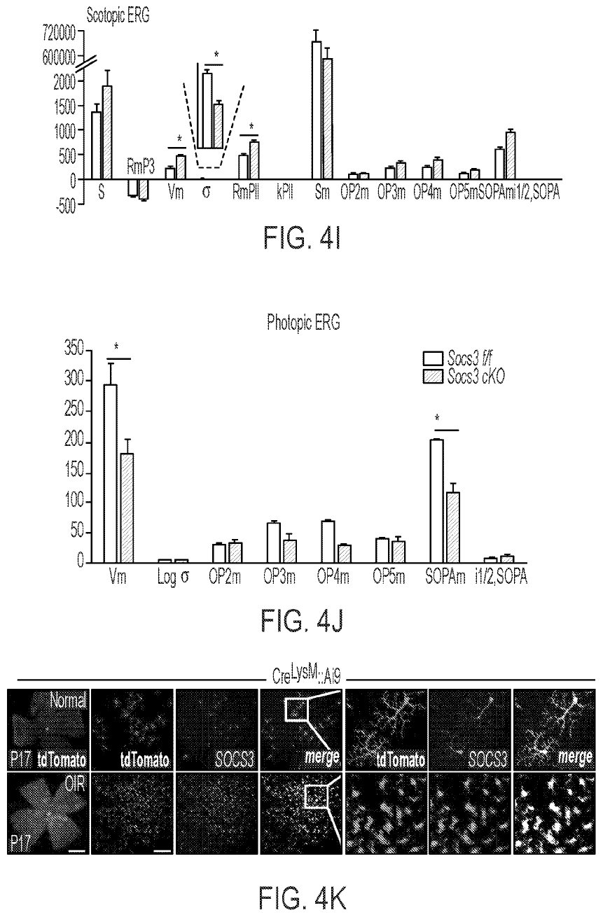

Next, expression of SOCS3 during OIR was assessed. Overall, SOCS3 expression was increased in myeloid cells during OIR, and was particularly increased in microglia and macrophages (FIG. 4A). These results were confirmed by immunofluorescent staining for SOCS3 (FIG. 4B). Mice with overexpression (OE) or knockdown (KO) of SOCS3 were then developed and OIR was induced. The degree of neovascularization in the mice was observed to be inversely correlated to with SOCS3 expression (FIGS. 4C-4F). Furthermore, the organization of blood vessels in the retina was observed to be disrupted during OIR and was further exacerbated by loss of SOCS3 function (FIGS. 4G and 4H). Finally, loss of SOCS3 function during OIR was associated with impaired vision, as determined by quantifying the loss of various photoreceptor cell types (FIGS. 4I and 4J). These results confirm the protective effect of SOCS3 in the retina against neovascularization.

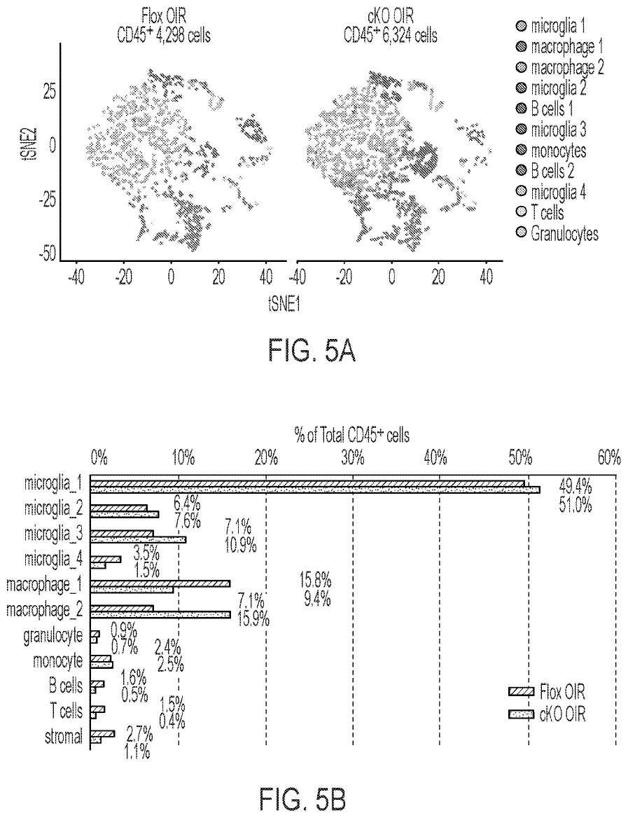

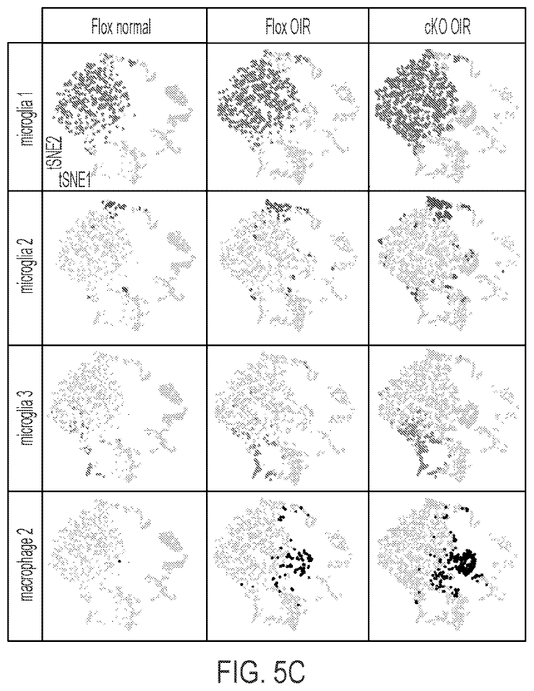

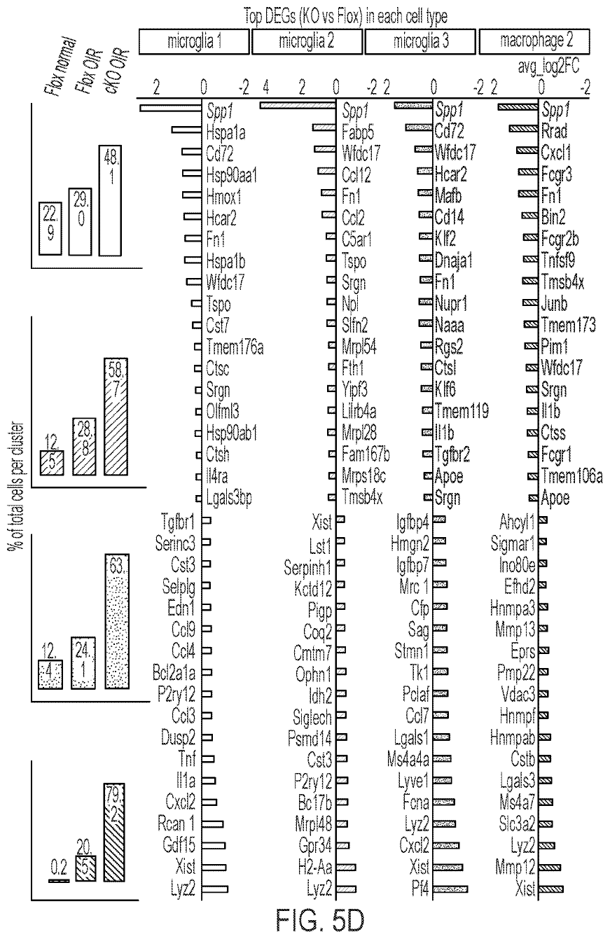

To better understand the function of SOCS3 function, differences between mice with SOCS3 knockdown (KO) and mice with transiently inactivated SOCS3 (Flox) was assessed. Mice without SOCS3 demonstrated a substantial increase in retinal myeloid cells during OIR as compared to mice with inactivated SOCS3 (FIGS. 5A and 5B). Notably, retinal macrophages and microglia were both increased in SOCS3 KO mice during OIR (FIG. 5C). Single cell sequencing of retinal macrophages and microglia from KO mice revealed considerable upregulation and downregulation of various genes in response to loss of SOCS3 function, potentially indication factors that were not previously recognized to drive or suppress neovascularization (FIG. 5D).

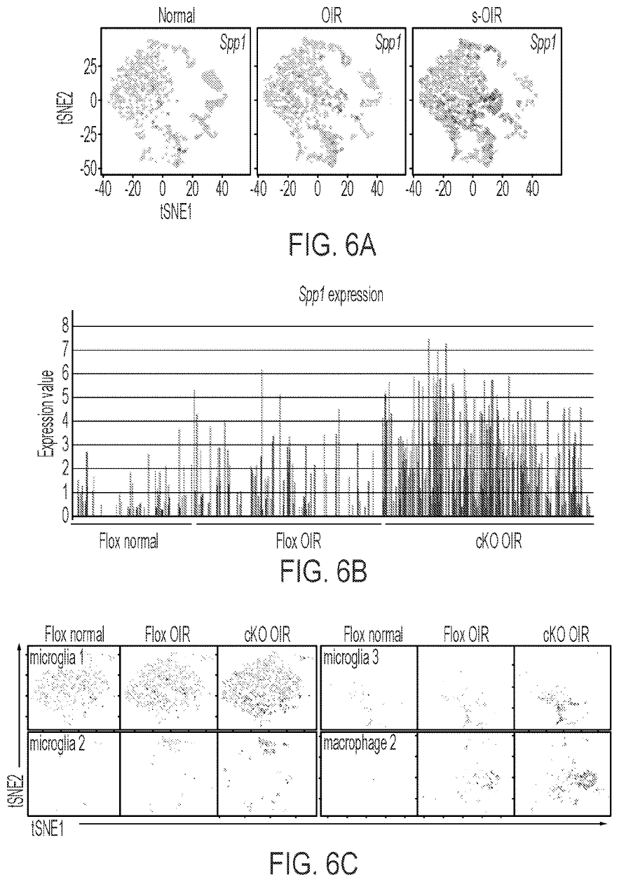

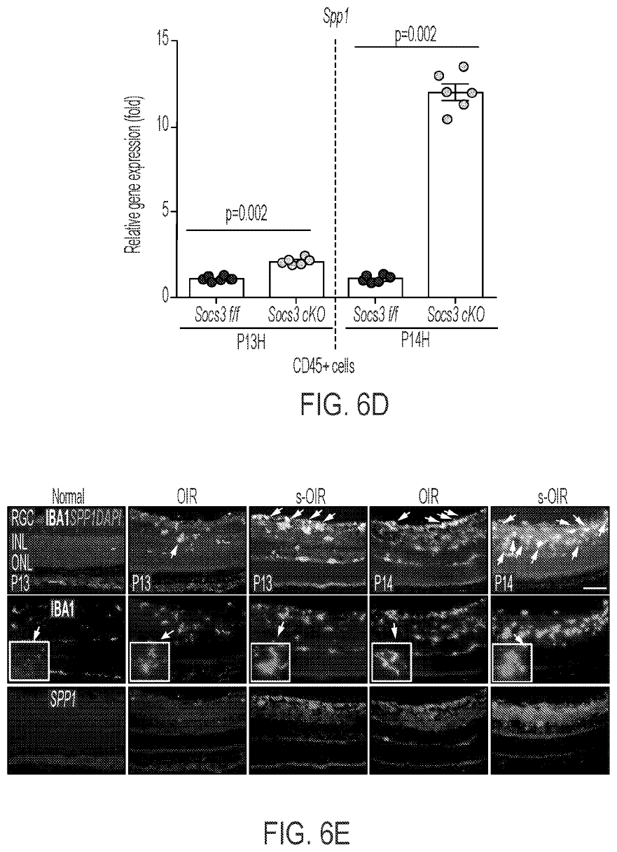

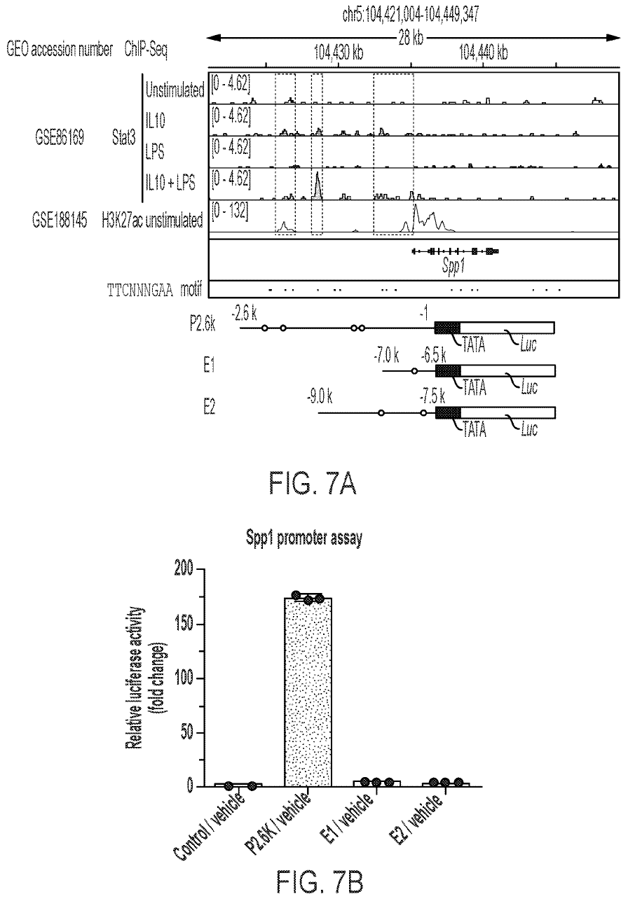

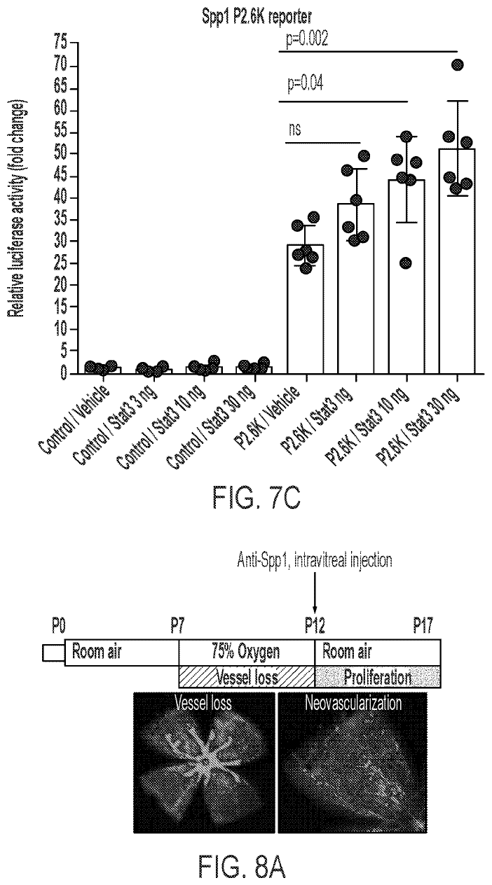

Secreted phosphoprotein 1 (Spp1) was observed to be the most upregulated factor in both macrophages and microglia from retinas of OIR mice lacking SOCS3 function, suggestion a major, previously unrecognized role in driving inflammation and neovascularization during retinopathy (FIGS. 6A and 6B). This observed upregulation was confirmed by both quantitative PCR (qPCR) and immunofluorescent staining (FIGS. 6D and 6E). It was then hypothesized that this upregulation may be mediated by STAT3, a transcription factor known to drive transcription of several pro-inflammatory factors. Therefore, a promoter and enhancer assay was devised with a luciferase reporter to test if alternate fragments of Spp1 would be transcriptionally targeted by STAT3 (FIG. 7A). Indeed, the Spp1 P2.6K reporter was observed to be transcriptionally regulated by STAT3 (FIG. 7B), and was further found to be upregulated in a dose-dependent fashion (FIG. 7C).

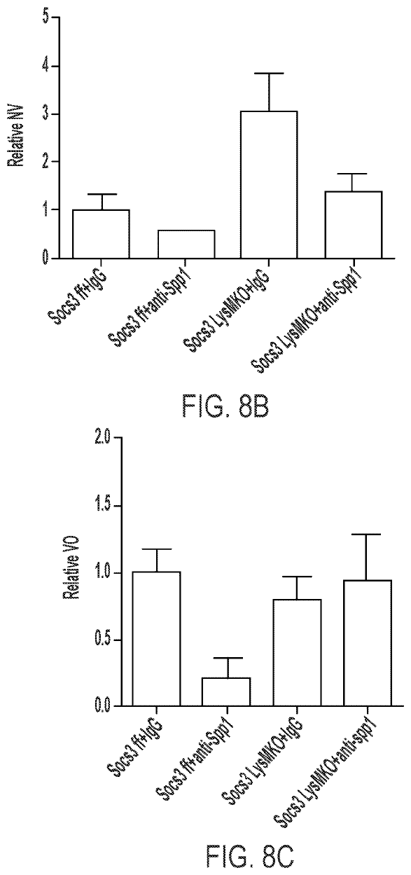

To confirm if Spp1 drives pathogenic neovascularization as a result of STAT3-mediated transcriptional regulation, OIR was induced in mice, however mice were treated with an antibody against Spp1 after removal from a high oxygen environment (FIG. 8A). As expected, treatment with an anti-Spp1 antibody substantially reduced retinal neovascularization during OIR, as compared to an IgG antibody control (FIG. 8B). Moreover, treatment with an anti-Spp1 antibody also reduced blood vessel loss in mice with functional SOCS3 during the initial phase of OIR (FIG. 8C). These results confirm that Spp1 promotes neovascularization during retinopathy and provide a promising proof-of-concept for the therapeutic ablation of Spp1 function in patients with ROP or another retinopathy.

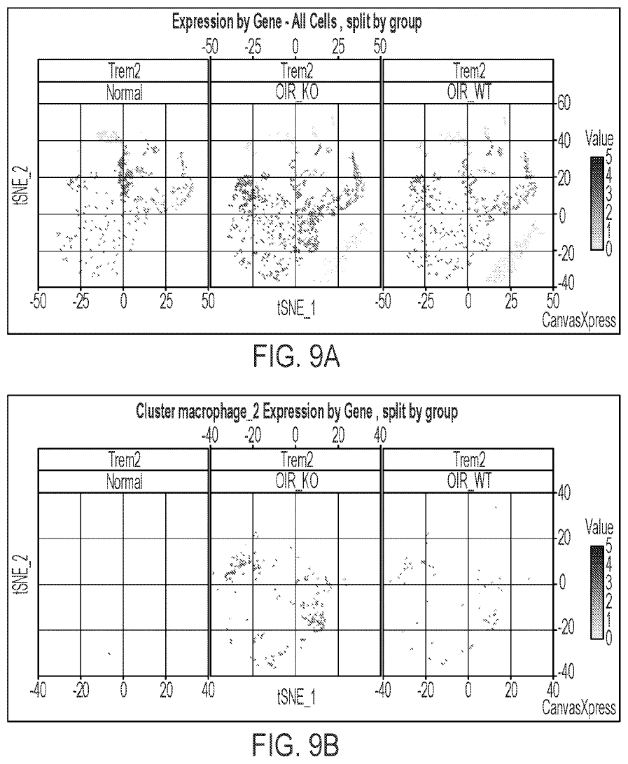

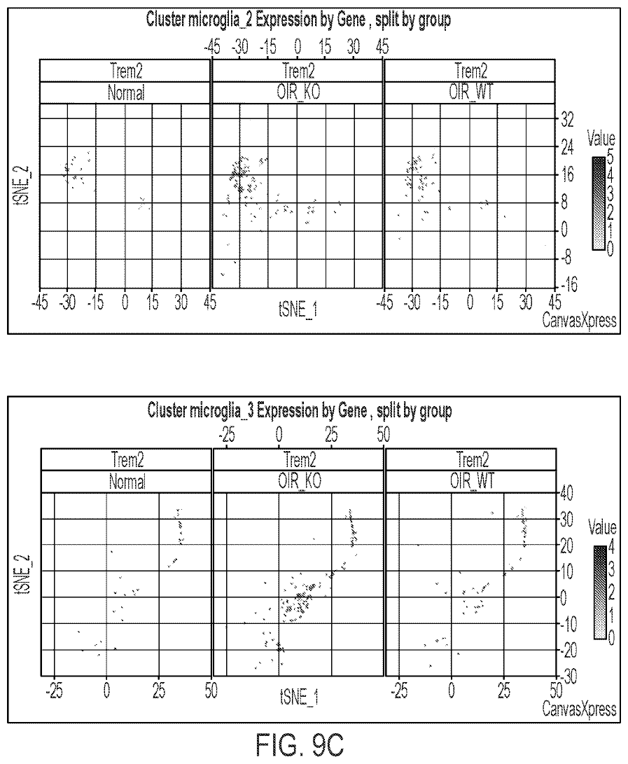

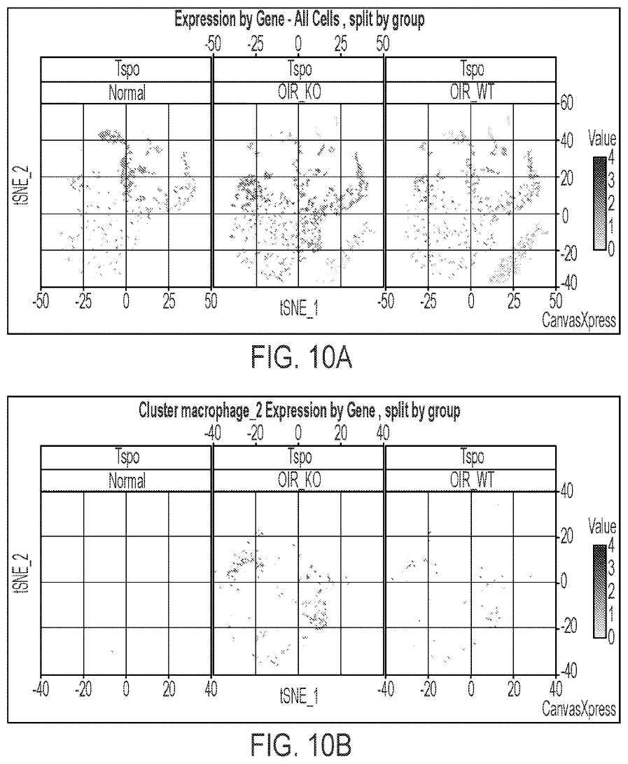

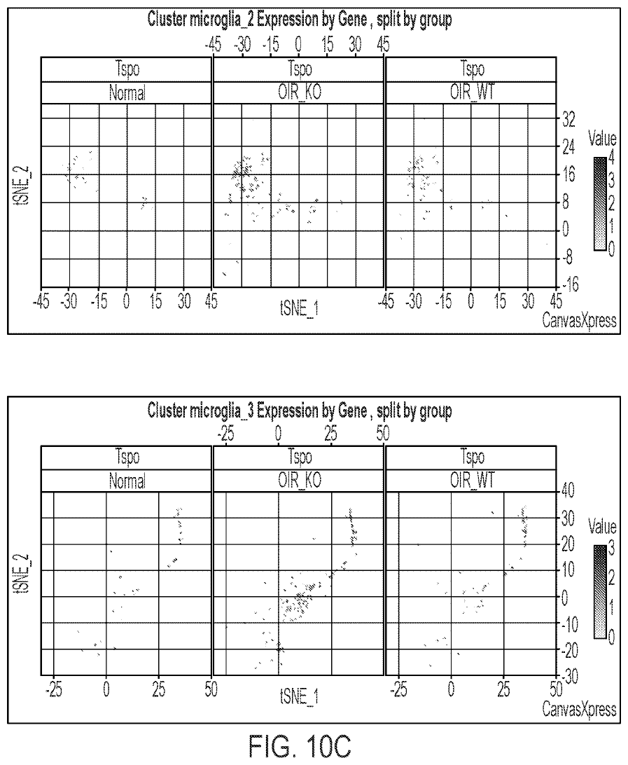

Given these promising results, further factors involved in the activation of retinal myeloid lineage cells were identified. Like Spp1, both triggering receptor expressed on myeloid cells 2 (Trem2) (FIGS. 9A-9C) and translocator protein (Tspo) (FIGS. 10A-10C) were further observed to be upregulated in the retinas of mice during OIR in the absence of SOCS3 function, suggesting similar roles in the promotion of pathogenic neovascularization. Transcription of these factors are likely to be further regulated by STAT3. Like Spp1, Trem2 and Tspo activity could be modulated by either agents that directly ablate the function of these factors, or by agents that ablate the function of STAT3.

### Methods

### Animals

All mouse studies were reviewed and approved by the Institutional Animal Care and Use Committee (IACUC) at the Boston Children's Hospital and all mouse experiments were performed following the guidance of The Association for Research in Vision and Ophthalmology (ARVO) for the ethical use of animals in ophthalmic and vision research. Socs3 flox (Socs3 f/f) mice (Yasukawa et al., 2003) were crossed with myeloid-specific LysM-Cre mice (Jackson Laboratory, stock #004781) to generate myeloid-specific Socs3 knockout mice (Socs3 cKO). Socs3 OE flox (Socs3 OE/OE) mice (Reed et al., 2010) were crossed with myeloid-specific LysM-Cre mice to generate Socs3 myeloid-specific overexpression mice (Socs3 cOE). Their littermate flox/flox mice were used as control mice. Both male and female mice were used for all experiments. C57BL/6J mice (stock #000664) and mTmG (membrane-associated tomato red and membrane-associated green fluorescent protein) reporter mice (stock #007576) were obtained from the Jackson Laboratory.

### Oxygen-Induced Retinopathy (OIR) and Vessel Quantification

OIR was carried out using neonatal mice as described previously (Smith et al., 1994). Briefly, mouse pups with nursing female mice were exposed to 75% oxygen from postnatal day (P) 7 to 12, then returned to room air until to P17 for phenotypical analysis and gene expression at indicated time. For phenotypical analysis, the retinas were collected at P17, dissected, and stained with fluorescent dye conjugated-Griffonia Simplicifolia Isolectin IB4 (Invitrogen) overnight and flat-mounted. The avascular (vasoobliteration, VO) and pathological neovascularization (NV) areas were quantified (Connor et al., 2009) by masked researchers using Image J (National Institutes of Health, imagej.nih.gov/ij/) and Adobe Photoshop (Adobe Systems). The exclusion criteria for the OIR mouse model was used as described previously. Mice with bodyweight less than 5 grams at P17 were excluded from the study (Stahl et al., 2010).

### Rna Isolation and Quantitative RT-PCR

Total RNA was extracted from mouse retina using Quick-RNA™ Miniprep Kit (Zymo Research, R1054). cDNA was synthesized using iScript™ cDNA Synthesis Kit (Bio-Rad, 1708890). Quantitative PCR (qPCR) was performed using SYBR Green qPCR Master Mix (Apex Bio, K1070).

### Immunohistochemistry

Immunostaining in retinas was performed as described (Sun et al., 2015a). Briefly, eyes were isolated from P17 mice with OIR, fixed and permeabilized. The flat-mounted retinas or cross sections were stained with antibodies, and imaged using a confocal laser scanning microscope (FV1000; Olympus and Zeiss 700).

### Immunoblot

A standard immunoblotting protocol was used. Briefly, 300 mM NaCl, 0.5% NP-40, 50 mM Tris-HCl pH7.4, 0.5 mM EDTA was used to lyse the retinas. Proteinase and phosphatase inhibitor cocktails were added.

Aortic Ring Sprouting Assay and Coculture with BMDM

Aortae from 2-month-old mice were dissected and cut into 1-mm-long pieces. Aortic rings were placed in growth factor-reduced Matrigel (BD Biosciences) and cultured for 5 days in EBM-2 medium (Lonza) (Li et al., 2014; Sapieha et al., 2011). Images of individual aortic explants were taken and the microvascular sprouting areas were quantified by measuring the area covered by outgrowth of the vascular sprouts with Image J. Bone marrow-derived macrophages were prepared as previously described (Weischenfeldt and Porse, 2008), and the culture medium was collected as a conditioned medium. On Day two, the conditioned medium from Socs3 cOE or Socs3 OE bone marrow-derived macrophage was added into aortic ring explants and replaced daily. Aortic ring explants were imaged using a Zeiss AxioOberver.Z1 microscope. ImageJ (National Institutes of Health) with a designed macro for SWIFT-Choroid quantification was used to quantify the sprouting area (Shao et al., 2013).

### Statistical Analysis

Statistical analyses were performed with GraphPad Prism (v5.0) (GraphPad Software, Inc., San Diego, CA). Results are presented as means+SEM. All data are representative of at least three independent experiments. Unpaired nonparametric Mann-Whitney Test was used for comparison. P values<0.05 were considered statistically significant.

### REFERENCES

1. Abcouwer, S. F. 2011. Neural inflammation and the microglial response in diabetic retinopathy. J Ocul Biol Dis Infor 4:25-33.
2. Aranda, J. V., F. Salomone, G. B. Valencia, and K. D. Beharry. 2017. Non-steroidal Anti-inflammatory Drugs in Newborns and Infants. Pediatr Clin North Am 64:1327-1340.
3. Arnold, C. E., C. S. Whyte, P. Gordon, R. N. Barker, A. J. Rees, and H. M. Wilson. 2013. A critical role for SOCS3 in promoting M1 macrophage activation and function in vitro and in vivo. Immunology
4. Binet, F., G. Mawambo, N. Sitaras, N. Tetreault, E. Lapalme, S. Favret, A. Cerani, D. Leboeuf, S. Tremblay, F. Rezende, A. M. Juan, A. Stahl, J. S. Joyal, E. Milot, R. J. Kaufman, M. Guimond, T. E. Kennedy, and P. Sapieha. 2013. Neuronal ER stress impedes myeloid-cell-induced vascular regeneration through IRE1alpha degradation of netrin-1. Cell metabolism 17:353-371.
5. Blencowe, H., S. Cousens, M. Z. Oestergaard, D. Chou, A. B. Moller, R. Narwal, A. Adler, C. Vera Garcia, S. Rohde, L. Say, and J. E. Lawn. 2012. National, regional, and worldwide estimates of preterm birth rates in the year 2010 with time trends since 1990 for selected countries: a systematic analysis and implications. Lancet 379:2162-2172.
6. Chambers, S. E., C. L. O'Neill, T. M. O'Doherty, R. J. Medina, and A. W. Stitt. 2013. The role of immune-related myeloid cells in angiogenesis. Immunobiology 218:1370-1375.
7. Checchin, D., F. Sennlaub, E. Levavasseur, M. Leduc, and S. Chemtob. 2006. Potential role of microglia in retinal blood vessel formation. Investigative ophthalmology & visual science 47:3595-3602.
8. Chen, L., P. Yang, and A. Kijlstra. 2002. Distribution, markers, and functions of retinal microglia. Ocul Immunol Inflamm 10:27-39.
9. Chen, M., A. Citil, F. McCabe, K. M. Leicht, J. Fiascone, C. E. Dammann, and O. Dammann. 2011. Infection, oxygen, and immaturity: interacting risk factors for retinopathy of prematurity. Neonatology 99:125-132.
10. Connor, K. M., N. M. Krah, R. J. Dennison, C. M. Aderman, J. Chen, K. I. Guerin, P. Sapicha, A. Stahl, K. L. Willett, and L. E. Smith. 2009. Quantification of oxygen-induced retinopathy in the mouse: a model of vessel loss, vessel regrowth and pathological angiogenesis. Nat Protoc 4:1565-1573.
11. Dammann, O. 2010. Inflammation and retinopathy of prematurity. Acta paediatrica 99:975-977.
12. Dammann, O., M. J. Brinkhaus, D. B. Bartels, M. Dordelmann, F. Dressler, J. Kerk, T. Dork, and C. E. Dammann. 2009. Immaturity, perinatal inflammation, and retinopathy of prematurity: a multi-hit hypothesis. Early Hum Dev 85:325-329.
13. Davies, M. H., J. P. Eubanks, and M. R. Powers. 2006. Microglia and macrophages are increased in response to ischemia-induced retinopathy in the mouse retina. Molecular vision 12:467-477.
14. Forrester, J. V., H. Xu, L. Kuffova, A. D. Dick, and P. G. McMenamin. 2010. Dendritic cell physiology and function in the eye. Immunological reviews 234:282-304.
15. Gao, X., Y. S. Wang, X. Q. Li, H. Y. Hou, J. B. Su, L. B. Yao, and J. Zhang. 2016. Macrophages promote vasculogenesis of retinal neovascularization in an oxygen-induced retinopathy model in mice. Cell and tissue research 364:599-610.
16. Grunin, M., S. Hagbi-Levi, and I. Chowers. 2014. The role of monocytes and macrophages in age-related macular degeneration. Advances in experimental medicine and biology 801:199-205.
17. Hartnett, M. E., and J. S. Penn. 2013. Mechanisms and management of retinopathy of prematurity. N Engl J Med 368:1162-1163.
18. Hong, H. K., H. J. Lee, J. H. Ko, J. H. Park, J. Y. Park, C. W. Choi, C. H. Yoon, S. J. Ahn, K. H. Park, S. J. Woo, and J. Y. Oh. 2014. Neonatal systemic inflammation in rats alters retinal vessel development and simulates pathologic features of retinopathy of prematurity. J Neuroinflammation 11:87.
19. Irmesi, R., M. A. Marcialis, J. V. Anker, and V. Fanos. 2014. Non-steroidal anti-inflammatory drugs (NSAIDs) in the management of patent ductus arteriosus (PDA) in preterm infants and variations in attitude in clinical practice: a flight around the world. Curr Med Chem 21:3132-3152.
20. Kambhampati, S. P., A. J. Clunies-Ross, I. Bhutto, M. K. Mishra, M. Edwards, D. S. McLeod. R. M. Kannan, and G. Lutty. 2015. Systemic and Intravitreal Delivery of Dendrimers to Activated Microglia/Macrophage in Ischemia/Reperfusion Mouse Retina. Investigative ophthalmology & visual science 56:4413-4424.
21. Kataoka, K., K. M. Nishiguchi, H. Kaneko, N. van Rooijen, S. Kachi, and H. Terasaki. 2011. The roles of vitreal macrophages and circulating leukocytes in retinal neovascularization. Investigative ophthalmology & visual science 52:1431-1438.
22. Klinger, G., I. Levy, L. Sirota, V. Boyko, L. Lerner-Geva, B. Reichman, and N. Israel Neonatal. 2010. Outcome of early-onset sepsis in a national cohort of very low birth weight infants. Pediatrics 125:0736-740.
23. Lang, R. A., and J. M. Bishop. 1993. Macrophages are required for cell death and tissue remodeling in the developing mouse eye. Cell 74:453-462.
24. Lee, J., and O. Dammann. 2012. Perinatal infection, inflammation, and retinopathy of prematurity. Semin Fetal Neonatal Med 17:26-29.
25. Li, J., C. H. Liu, Y. Sun, Y. Gong, Z. Fu, L. P. Evans, K. T. Tian, A. M. Juan, C. G. Hurst, A. Mammoto, and J. Chen. 2014. Endothelial TWIST1 Promotes Pathological Ocular Angiogenesis. Invest Ophthalmol Vis Sci 55:8267-8277.
26. Licbowitz, M., and R. I. Clyman. 2017. Prophylactic Indomethacin Compared with Delayed Conservative Management of the Patent Ductus Arteriosus in Extremely Preterm Infants: Effects on Neonatal Outcomes. J Pediatr 187:119-126 e111.
27. Lutty, G. A., T. Chan-Ling, D. L. Phelps, A. P. Adamis, K. I. Berns, C. K. Chan, C. H. Cole, P. A. D'Amore, A. Das, W. T. Deng. V. Dobson, J. T. Flynn, M. Friedlander, A. Fulton, W. V. Good, M. B. Grant, R. Hansen, W. W. Hauswirth, R. J. Hardy, D. R. Hinton, S. Hughes, D. S. McLeod, E. A. Palmer, A. Patz, J. S. Penn, B. J. Raisler, M. X. Repka, M. Saint-Genicz, L. C. Shaw, D. T. Shima, B. T. Smith, L. E. Smith, S. G. Tahija, W. Tasman, and M. T. Trese. 2006. Proceedings of the Third International Symposium on Retinopathy of Prematurity: an update on ROP from the lab to the nursery (November 2003, Anaheim, California). Molecular vision 12:532-580.
28. McMenamin, P. G. 1999. Dendritic cells and macrophages in the uveal tract of the normal mouse eye. Br J Ophthalmol 83:598-604.
29. Melamed, N., J. Shah, A. Soraisham, E. W. Yoon, S. K. Lee, P. S. Shah, and K. E. Murphy. 2015. Association Between Antenatal Corticosteroid Administration-to-Birth Interval and Outcomes of Preterm Neonates. Obstet Gynecol 125:1377-1384.
30. Mitamura, Y., C. Harada, and T. Harada. 2005. Role of cytokines and trophic factors in the pathogenesis of diabetic retinopathy. Curr Diabetes Rev 1:73-81.
31. Moscuzza, F., F. Belcari, V. Nardini, A. Bartoli, C. Domenici, A. Cuttano, P. Ghirri, and A. Boldrini. 2011. Correlation between placental histopathology and fetal/neonatal outcome: chorioamnionitis and funisitis are associated to intraventricular haemorrage and retinopathy of prematurity in preterm newborns. Gynecol Endocrinol 27:319-323.
32. Network, S. S. G.o.t.E. K. S. N. N. R., W. A. Carlo, N. N. Finer, M. C. Walsh, W. Rich, M. G. Gantz, A. R. Laptook, B. A. Yoder, R. G. Faix, A. Das, W. K. Poole, K. Schibler, N. S.

Newman, N. Ambalavanan, I. D. Frantz, 3rd, A. J. Piazza, P. J. Sanchez, B. H. Morris, N. Laroia, D. L. Phelps, B. B. Poindexter, C. M. Cotten, K. P. Van Meurs, S. Duara, V. Narendran, B. G. Sood, T. M. O'Shea, E. F. Bell, R. A. Ehrenkranz, K. L. Watterberg, and R. D. Higgins. 2010. Target ranges of oxygen saturation in extremely preterm infants. N Engl J Med 362:1959-1969.
33. Polam, S., A. Koons, M. Anwar, S. Shen-Schwarz, and T. Hegyi. 2005. Effect of chorioamnionitis on neurodevelopmental outcome in preterm infants. Arch Pediatr Adolesc Med 159:1032-1035.
34. Qin, H., A. T. Holdbrooks, Y. Liu, S. L. Reynolds, L. L. Yanagisawa, and E. N. Benveniste. 2012. SOCS3 deficiency promotes M1 macrophage polarization and inflammation. Journal of immunology 189:3439-3448.
35. Reed, A. S., E. K. Unger, L. E. Olofsson, M. L. Piper, M. G. Myers, Jr., and A. W. Xu. 2010. Functional role of suppressor of cytokine signaling 3 upregulation in hypothalamic leptin resistance and long-term energy homeostasis. Diabetes 59:894-906.
36. Ritter, M. R., E. Banin, S. K. Moreno, E. Aguilar, M. I. Dorrell, and M. Friedlander. 2006. Myeloid progenitors differentiate into microglia and promote vascular repair in a model of ischemic retinopathy. The Journal of clinical investigation 116:3266-3276.
37. Santos, A. M., R. Calvente, M. Tassi, M. C. Carrasco, D. Martin-Oliva, J. L. Marin-Teva, J. Navascucs, and M. A. Cuadros. 2008. Embryonic and postnatal development of microglial cells in the mouse retina. The Journal of comparative neurology 506:224-239. Sapicha, P., A. Stahl, J. Chen, M. R. Seaward, K. L. Willett, N. M. Krah, R. J. Dennison, 38. K. M. Connor, C. M. Aderman, E. Liclican, A. Carughi, D. Perelman, Y. Kanaoka, J. P. Sangiovanni, K. Gronert, and L. E. Smith. 2011. 5-Lipoxygenase Metabolite 4-HDHA Is a Mediator of the Antiangiogenic Effect of {omega}-3 Polyunsaturated Fatty Acids. Sci Transl Med 3: 69ra12.
39. Shao, Z., M. Friedlander, C. G. Hurst, Z. Cui, D. T. Pei, L. P. Evans, A. M. Juan, H. Tahiri, F. Duhamel, J. Chen, P. Sapicha, S. Chemtob, J. S. Joyal, and L. E. Smith. 2013. Choroid sprouting assay: an ex vivo model of microvascular angiogenesis. PloS one 8: e69552.
40. Smith, L. E., E. Wesolowski, A. Mclellan, S. K. Kostyk, R. D'Amato, R. Sullivan, and P. A. D'Amore. 1994. Oxygen-induced retinopathy in the mouse. Investigative ophthalmology & visual science 35:101-111.
41. Spence, S., A. Fitzsimons, C. R. Boyd, J. Kessler, D. Fitzgerald, J. Elliott, J. N. Gabhann, S. Smith, A. Sica, E. Hams, S. P. Saunders, C. A. Jefferies, P. G. Fallon, D. F. McAuley, A. Kissenpfennig, and J. A. Johnston. 2013. Suppressors of cytokine signaling 2 and 3 diametrically control macrophage polarization. Immunity 38:66-78.
42. Stahl, A., J. Chen, P. Sapicha, M. R. Scaward, N. M. Krah, R. J. Dennison, T. Favazza, F. Bucher, C. Lofqvist, H. Ong, A. Hellstrom, S. Chemtob, J. D. Akula, and L. E. Smith. 2010. Postnatal weight gain modifies severity and functional outcome of oxygen-induced proliferative retinopathy. The American journal of pathology 177:2715-2723.
43. Stahl, A., J. S. Joyal, J. Chen, P. Sapicha, A. M. Juan, C. J. Hatton, D. T. Pei, C. G. Hurst, M. R. Scaward, N. M. Krah, R. J. Dennison, E. R. Greene, E. Boscolo, D. Panigrahy, and L. E. Smith. 2012. SOCS3 is an endogenous inhibitor of pathologic angiogenesis. Blood 120:2925-2929.
44. Stefater, J. A., 3rd, I. Lewkowich, S. Rao, G. Mariggi, A. C. Carpenter, A. R. Burr, J. Fan, R. Ajima, J. D. Molkentin, B. O. Williams, M. Wills-Karp, J. W. Pollard, T. Yamaguchi, N. Ferrara, H. Gerhardt, and R. A. Lang. 2011a. Regulation of angiogenesis by a non-canonical Wnt-Flt1 pathway in myeloid cells. Nature 474:511-515.
45. Stefater, J. A., 3rd, S. Ren, R. A. Lang, and J. S. Duffield. 2011b. Metchnikoff's policemen: macrophages in development, homeostasis and regeneration. Trends Mol Med 17:743-752.
46. Sun, Y., M. Ju. Z. Lin, T. W. Fredrick, L. P. Evans, K. T. Tian, N. J. Saba, P. C. Morss, W. T. Pu. J. Chen, A. Stahl, J. S. Joyal, and L. E. Smith. 2015a. SOCS3 in retinal neurons and glial cells suppresses VEGF signaling to prevent pathological neovascular growth. Sci Signal 8: ra94.
47. Sun, Y., C. H. Liu, J. P. SanGiovanni, L. P. Evans, K. T. Tian, B. Zhang, A. Stahl, W. T. Pu, T. M. Kamenecka, L. A. Solt, and J. Chen. 2015b. Nuclear receptor RORalpha regulates pathologic retinal angiogenesis by modulating SOCS3-dependent inflammation. Proceedings of the National Academy of Sciences of the United States of America 112:10401-10406.
48. Tasman, W., A. Patz, J. A. McNamara, R. S. Kaiser, M. T. Trese, and B. T. Smith. 2006. Retinopathy of prematurity: the life of a lifetime disease. Am J Ophthalmol 141:167-174.
49. Tolsma, K. W., E. N. Allred, M. L. Chen, J. Duker, A. Leviton, and O. Dammann. 2011. Neonatal bacteremia and retinopathy of prematurity: the ELGAN study. Archives of ophthalmology 129:1555-1563.
50. Tsutsumi, C., K. H. Sonoda, K. Egashira, H. Qiao, T. Hisatomi, S. Nakao, M. Ishibashi, I. F. Charo. T. Sakamoto, T. Murata, and T. Ishibashi. 2003. The critical role of ocular-infiltrating macrophages in the development of choroidal neovascularization. Journal of leukocyte biology 74:25-32.
51. Weischenfeldt, J., and B. Porse. 2008. Bone Marrow-Derived Macrophages (BMM): Isolation and Applications. CSH Protoc 2008: pdb prot5080.
52. Yang, P., A. F. de Vos, and A. Kijlstra. 1997. Macrophages and MHC class II positive cells in the choroid during endotoxin induced uveitis. Br J Ophthalmol 81:396-401.
53. Yasukawa, H., M. Ohishi, H. Mori, M. Murakami, T. Chinen, D. Aki, T. Hanada, K. Takeda, S. Akira, M. Hoshijima, T. Hirano, K. R. Chien, and A. Yoshimura. 2003. IL-6 induces an anti-inflammatory response in the absence of SOCS3 in macrophages. Nature immunology 4:551-556.
54. Zhou, Y., S. Yoshida, S. Nakao, T. Yoshimura, Y. Kobayashi, T. Nakama, Y. Kubo, K. Miyawaki, M. Yamaguchi, K. Ishikawa, Y. Oshima, K. Akashi, and T. Ishibashi. 2015. M2 Macrophages Enhance Pathological Neovascularization in the Mouse Model of Oxygen-Induced Retinopathy. Investigative ophthalmology & visual science 56:4767-4777.

### Example 2: Neovascularization Associated Microglia Controls Retinal Angiogenesis Via SOCS3/SPP1

Pathological ocular angiogenesis has been associated with inflammatory changes. Yet, the cellular and molecular mechanisms governing immune-vascular crosstalk during ocular neovascularization (NV) formation remain largely unknow. The present disclosure identified NV-associated microglia and macrophages (NVAMs) during retinopathy in mouse models of retinal angiogenesis. NVAMs were controlled by suppressor of cytokine signaling 3 (SOCS3) and secreted phosphoprotein 1 (Spp1) expression was highly upregulated in NVAMs from myeloid SOCS3 deficient retinas compared to control mice. Spp1 expression was colocalized with microglia and macrophage marker genes in the NV areas in angiogenesis mouse models. In addition, Spp1 was a direct target gene of signal transducer and activator of transcription 3. Moreover, double knockout of Spp1 and myeloid Socs3 reversed myeloid Socs3 deletion-induced NV and blocking SPP1 reduced myeloid SOCS3 deficient-induced NV. Thus, the SOCS3/SPP1 pathway tightly controls inflammation and pathological angiogenesis by regulating NVAMs during NV development in retinopathies.

Ocular neovascularization (NV) is seen in vascular eye diseases, such as retinopathy of prematurity (ROP) and neovascular age-related macular degeneration (AMD) and is a major cause of blindness¹⁻⁴. All neonates have immature immune systems, particularly premature neonates with low birth weight, who are particularly susceptible to infection. Preterm newborns exposed to infection have a high risk for ROP⁵⁻⁸. Infectious organisms and/or their microbial products stimulate the production of proinflammatory cytokines, which supports an association of increased systemic cytokines with an increased risk for ROP⁹. In a large cohort study, infants with early-onset sepsis showed a higher risk for severe ROP¹⁰. In the Extremely Low Gestational Age Newborns Study, late-onset neonatal sepsis is associated with an increased risk for ROP⁵.

Infection and inflammation are more frequently observed clinically, promoting the need for a deeper understanding of the pathogenesis of ROP⁹. Inflammation and immune dysfunction are clearly associated with ROP^{5, 6, 9, 11-13}, but the link has not been well studied and approaches for controlling such inflammation are not yet clear. Current anti-inflammatory therapies, such as corticosteroids, may benefit ROP¹⁴. Non-steroidal anti-inflammatory drugs are commonly used in newborns for pharmacologic closure of the patent ductus arteriosus¹⁵, but whether or not these drugs prevent ROP is unclear¹⁶. Moreover, immune cells have been reported to modulate retinal inflammation and pathological NV¹⁷⁻¹⁹.

Hypoxia attracts macrophages into hypoxic areas^{20,21}, where the hypoxia-activated macrophages and microglia release not only proinflammatory but also angiogenic cytokines. Myeloid cells, particularly infiltrating macrophages and local microglia have been implicated in the pathogenesis of vascular eye diseases²²⁻²⁴. Both recruited and blood-derived macrophages have been shown to participate in the regulation of retinal angiogenesis in the classical mouse model of ocular NV, the oxygen-induced retinopathy (OIR) model²⁵⁻²⁸. Proinflammatory cytokines and growth factors are released by myeloid cells to regulate retinal vessel development^{29, 30}, remodeling³¹, and repair^{25, 32-34} Microglia, the primary resident retina immune cells, are rapidly activated after an insult^{35, 36} and directly modulate angiogenesis^{25, 30}. Myeloid cells in circulating blood³⁷⁻³⁹ have been characterized in mouse retinal flat-mounts and sections³⁸. However, both immune-vascular crosstalk and the underlying molecular mechanism in ocular NV remain largely unknown.

Suppressor of cytokine signaling 3 (SOCS3) can regulate tissue inflammation and cytokine secretion⁴⁰⁻⁴², as well as control pathological ocular angiogenesis⁴³⁻⁴⁶. Endothelial SOCS3 deficiency increases retinal NV by inhibiting the signal transducer and activator of transcript 3 (STAT3) and mammalian target of rapamycin (mTOR) pathways in OIR mice⁴³. It has previously been reported that neuronal and glial SOCS3 inhibits pathological retinal angiogenesis in OIR mice by suppressing vascular endothelial growth factor A (VEGFA)++, and that suppression of SOCS3 abolishes the anti-inflammatory and vaso-protective effects of retinoic-acid-receptor-related orphan receptor alpha deficiency in OIR mice⁴⁵. Hombrebueno et al. reported that myeloid SOCS3 deficiency enhanced NV in OIR mice⁴⁶. The present disclosure demonstrates the critical role of myeloid SOCS3 in the OIR model by both overexpressing and deletion of SOCS3 in mouse immune cells of myeloid origin and further investigated the downstream soluble proteins of the SOCS3/STAT3 pathway. NV-associated microglia and macrophages (NVAMs) were identified during OIR using single-cell RNA sequencing (scRNA-seq) on immune cells isolated from myeloid SOCS3-deficient retinas and control retinas, both with or without OIR. The data showed that secreted phosphoprotein 1 (SPP1), also known as Osteopontin (OPN), was highly induced in NVAMs in myeloid SOCS3 deficient OIR retinas. The data also showed that Spp1 transcription was directly regulated by STAT3.

SPP1 is a cytokine that upregulates expression of interferon-gamma and interleukin-12⁴⁷, and is expressed by microglia and macrophage subsets, T cells, and other immune cells to modulate various macrophage functions, including phagocytosis. In choroidal neovascular membranes surgically extracted from human AMD patients, SPP1 expression is increased and numerous SPP1 positive myeloid cells are observed48. The data showed that Spp1 was highly induced in the NV areas in OIR mice, particularly the OIR mice lacking myeloid SOCS3. NVAMs were identified in mice with OIR and myeloid SOCS3 deficient OIR mice using scRNA-seq analysis. Spp1 expression was markedly increased in these NVAMs in OIR retinas lacking myeloid SOCS3. Importantly, double knockout of Spp1 and myeloid Socs3 suppressed myeloid SOCS3 deficiency-induced NV. These findings suggest that SPP1-expressing NVAMs may contribute to the development of ocular NV.

The results show that myeloid SOCS3 controls pathological retinal NV in OIR mice by modulating SPP1-expressing NVAMs, demonstrating the important role of NVAMs in controlling immune-vascular crosstalk during the development of retinal angiogenesis via the SOCS3/SPP1 pathway.

### Myeloid Lineage Cell Accumulation During the Development of NV

In OIR retinas, more ionized calcium-binding adaptor molecule 1 positive (IBA1⁺) activated microglia and macrophages were observed around NV (labeled by lectin) compared to normal retinas (FIG. 1H). To visualize the involvement of myeloid lineage cells during the development of OIR, myeloid-specific reporter mice—membrane-targeted two-color (membrane-localized tdTomato and membrane-localized enhanced green fluorescent protein, GFP) fluorescent mice driven by myeloid-specific Cre mice (Cre^{LysM}::mTmG)—were generated to identify myeloid lineage cells⁴⁹. The mice were subjected to OIR, and the whole retinas were examined using two-photon microscopy (FIG. 1B). Representative images of the 3D reconstruction of retinal whole mounts with OIR at P17 are shown in FIG. 1C. The location and morphology of myeloid lineage cells are shown in FIGS. 1D-1E. These data indicate that myeloid cells may accumulate at sites of retinal NV formation in OIR. To further examine the interaction between myeloid lineage cells and NV, confocal images of OIR retinas were taken with high magnification (FIGS. 1F-1G). Myeloid lineage cells were recruited to the NV areas labeled by endothelial cell marker CD31 (FIG. 1F). Some of these myeloid lineage cells were IBA1⁺ microglia and macrophages (FIG. 1G). These data highlight the activation and recruitment of myeloid lineage cells, specifically activated microglia and macrophages at sites of retinal NV formation and suggest a potential role in the OIR retinal phenotype.

Myeloid SOCS3-Regulated NV in Mice with OIR

It had previously been reported that myeloid SOCS3 may contribute to the formation of choroidal NV (CNV) 49; however, the present disclosure examined whether myeloid SOCS3 also contributes to the formation of retinal NV in the OIR mouse model. SOCS3 expression was induced in CD11b-expressing myeloid cells (arrows) compared to normal retina at P14 (FIG. 4B). To further visualize SOCS3 expression during OIR, myeloid-specific Cre^{LysM}-driven Ai9 tandem dimer Tomato (tdTomato) reporter mice (Cre^{LysM}::Ai9) were generated and subsequently examined to determine the expression of SOCS3 in the Cre^{LysM}::Ai9 mice with or without OIR (FIG. 4K). More myeloid lineage cells labelled by tdTomato were observed in OIR retina compared to normal controls, and SOCS3 was induced in myeloid cells. The morphology of myeloid lineage cells was changed from ramified shape to activated or amoeboid shapes in OIR mice. These results demonstrate that SOCS3 was induced in myeloid cells, specifically in those activated microglia and macrophages that accumulate in the OIR retinas during NV development.

Next, myeloid-specific Socs3 knockout mice (Socs3 cKO) driven by a myeloid-specific Cre^{LysM} were generated and showed that loss of myeloid Socs3 significantly increased NV (FIGS. 4C-4D), consistent with the results reported from Xu's group⁴⁶. Socs3 overexpression mice (Socs3 cOE) were also generated and, conversely, overexpression of Socs3 dramatically suppressed retinal NV without influencing retinal vaso-obliteration (VO), further confirming the critical role of SOCS3 in directly affecting retinal NV development (FIGS. 4E-4F). In addition, the data represented that retinal vascular tortuosity in OIR mice was worsened by myeloid SOCS3 deficiency (FIG. 4G). Retinal vascular tortuosity has been reported in preterm infants, especially in infants with severe ROP⁵⁰, suggesting that a reduced level of myeloid SOCS3 expression may increase the risk of serve ROP. These findings suggest that myeloid SOCS3 deficiency led to increased NV and worsened tortuosity in OIR mice. These mice could be used to model more advanced stage ROP requiring treatment for mechanistic studies.

### Distinct Immune Cell Types of the Mouse Retina

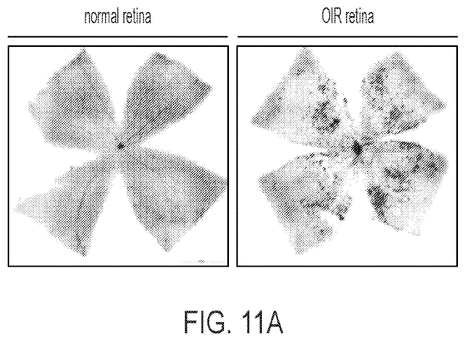

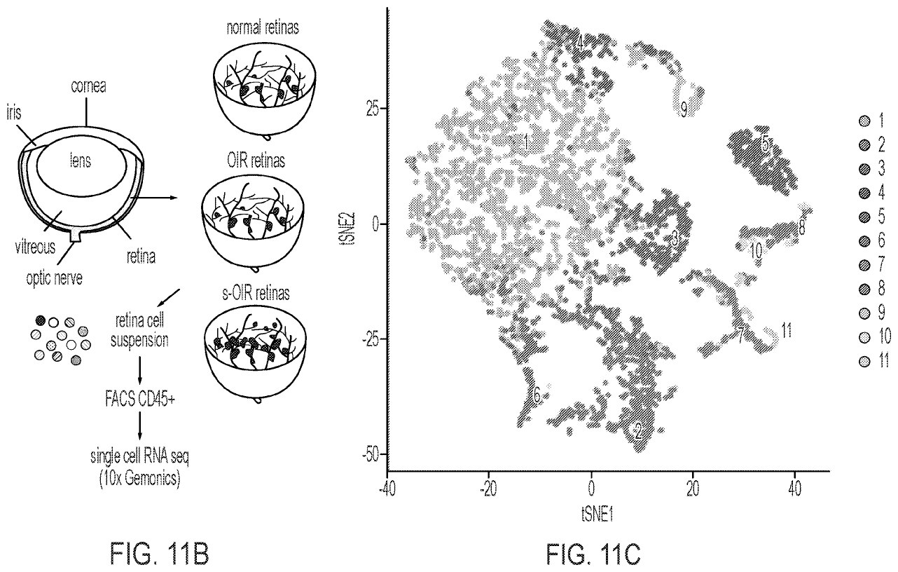

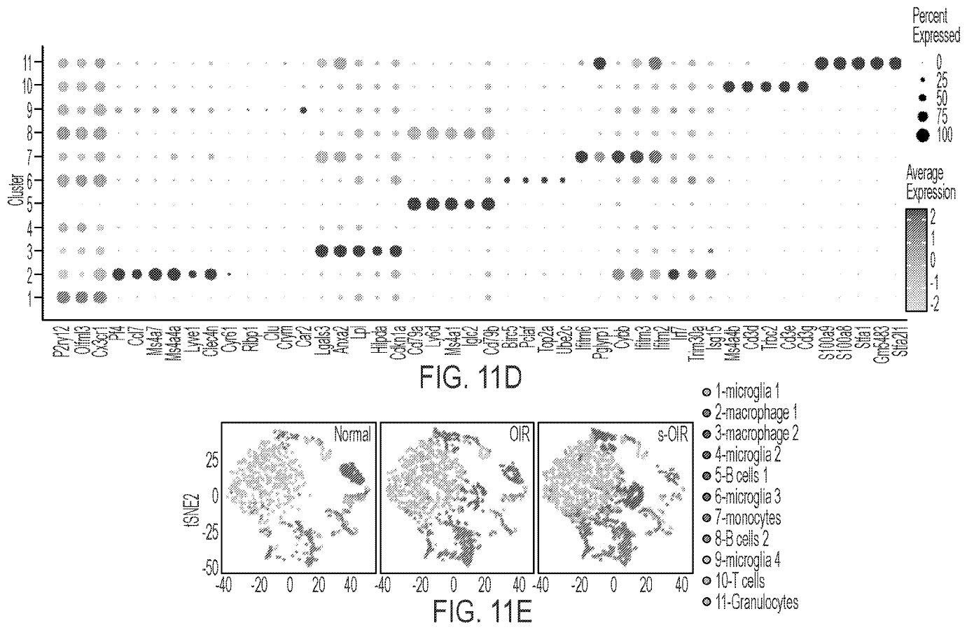

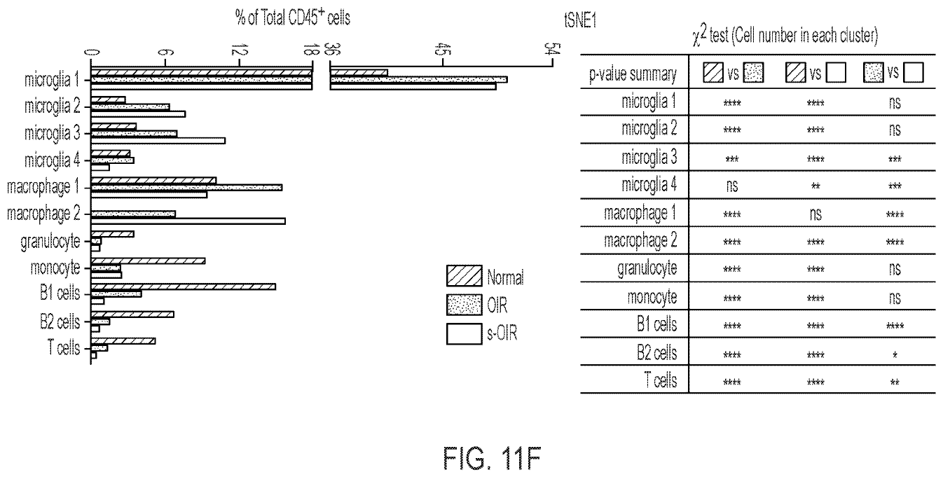

To investigate the specific populations of myeloid lineage cells that contribute to the NV development, the scRNA-seq was performed on normal retinas, OIR retinas, and retinas with severe OIR phenotype from mice lacking myeloid SOCS3. Based on a previous report51, less than 1% of the total cells in the retina are immune cells. Therefore, retinal immune cells pooled from multiple retinas were enriched by cell sorting with CD45 antibody for downstream scRNA-seq (FIGS. 11A-11B). Based on the scRNA-seq data, 11 distinct clusters were detected as shown in the t-distributed stochastic neighbor embedding (t-SNE) plots (FIG. 11C). The clusters were distinguished by differential expression of immune gene markers (FIG. 11D), which were mapped into different immune cell types using ImmGen ULI RNA-Seq database (FIG. 2E). Ten out of 11 clusters expressed different levels of microglia marker genes, such as Cx3cr1, P2ry12, and Olfm13. Two clusters (2 and 3) with reduced levels of microglia markers were identified as macrophage-like microglia and named as macrophage 1 and 2 based on marker genes, such as Pf4, Cc17, Ms4a7, Ms4a4a, Lyve1, Clec4n, Isg15, Trim30a, and Irf7 for macrophage 1, and Lgals3, Anxa2, Lpl, Hilpda, and Cdknla for macrophage 2. Four clusters (1, 4, 6, and 9) were identified as microglia 1, 2, 3, and 4, respectively based on marker genes such as Cx3cr1, P2ryl2, and Olfml3. In addition, Cluster 7 was identified as monocytes using markers including Ifitm2, Ifitm3, Ifitm6P, Glyrp1, and Cybb. Clusters 5 and 8 were identified as B cells (B cells 1 and 2) using markers including Cd79a, Cd79b, Cy6d, Ms4al, and Iglc2. Cluster 8 has reduced level of gene markers for B cells and microglia markers, which was annotated as B cell 2. Cluster 10 was identified as T cells using marker genes Cd3d, Cd3e, Cd3g, and Trbc2. Cluster 11 was identified as granulocytes using marker genes S100a9, S100a8, Stfal, Gm5483, and Stf211. Altogether, a total of 11 distinct clusters of CD45⁺ cells were identified in the mouse retina at P13 (FIG. 11E).

### Nvams Accumulation During OIR Via SOCS3 Control

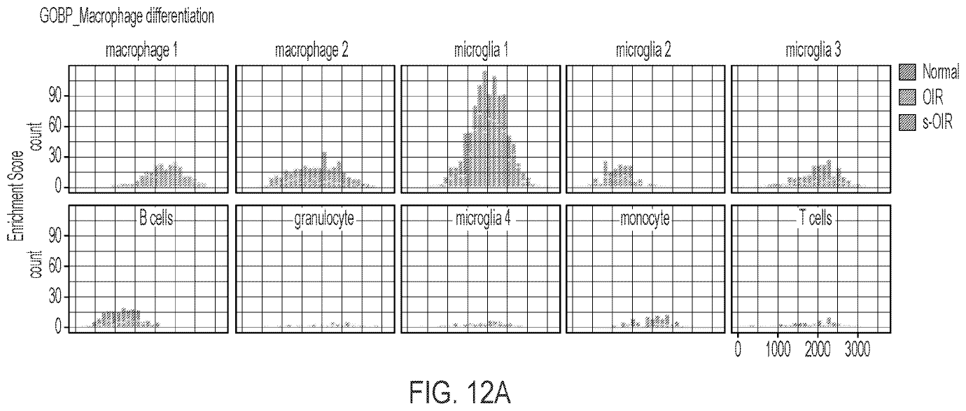

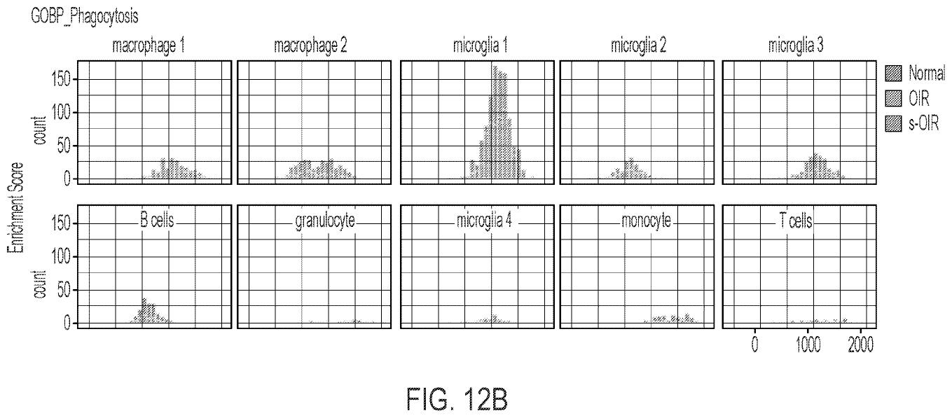

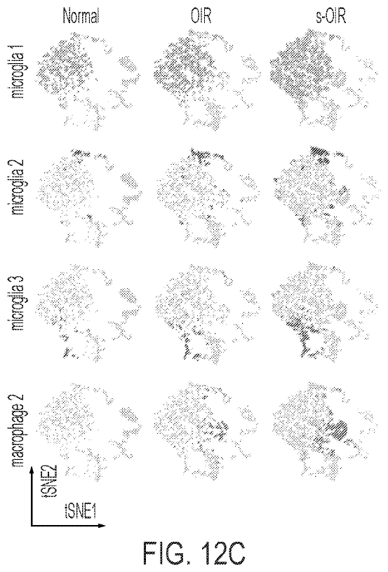

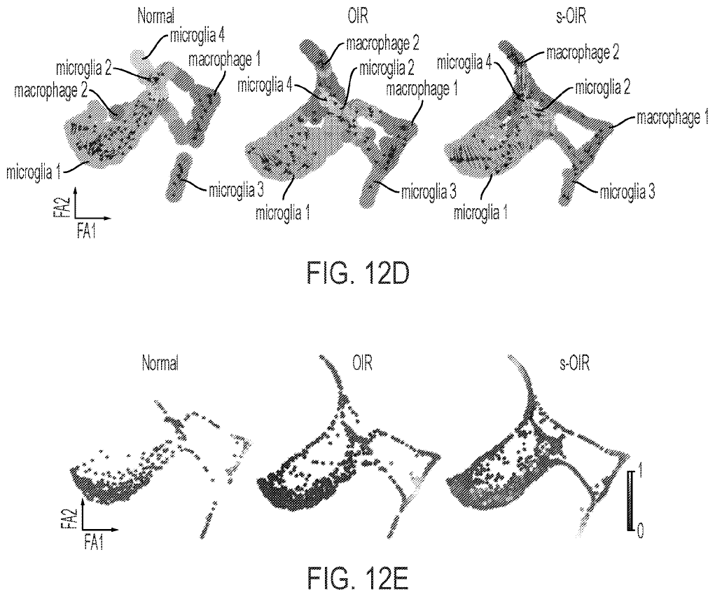

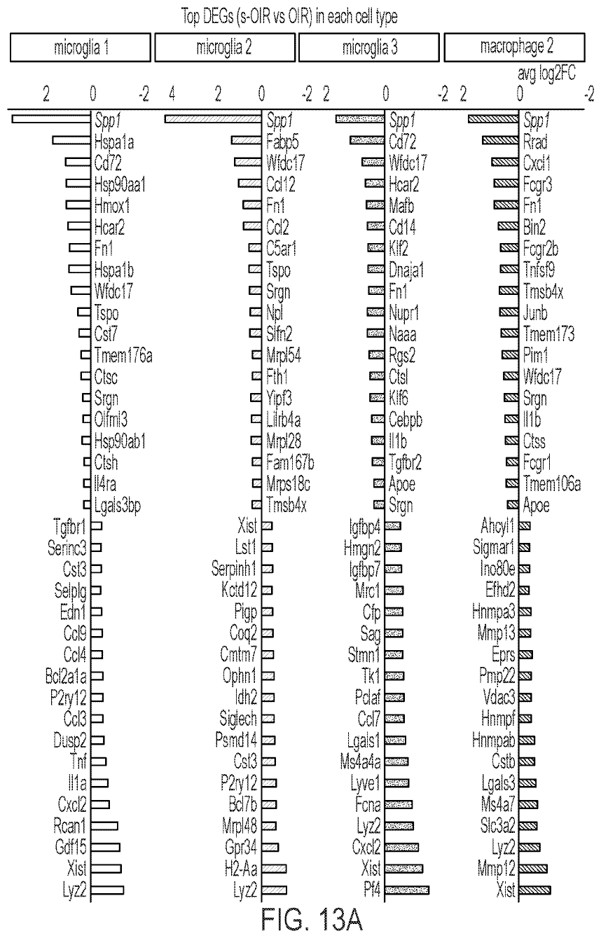

The cell number and the percentage of each cluster of CD45⁺ cells were compared in the following groups: normal, OIR, and the OIR mice lacking myeloid SOCS3. The cell number in each cell type showed significant difference in OIR retinas (except microglia 4) and in myeloid SOCS3 deficient OIR retinas (except macrophage 1) compared to normal retinas (FIG. 11F). In OIR retinas compared to normal retinas, the percentages of total CD45⁺ cells of microglia 1, 2, and 3 were increased by 0.24-, 1.25-, and 0.91-folds, respectively, but microglia 4 only increased by 0.08-fold, and macrophages 1 and 2 were increased by 0.53- and 82.12-folds, respectively. In myeloid SOCS3 deficient OIR retina versus normal retinas, microglia 1, 2, and 3 were increased by 0.22-, 1.72-, and 1.97-folds, respectively, but not microglia 4 (reduced by 0.53-fold) and macrophages 1 (reduced by 0.07-fold). The macrophage 2 cluster was increased by 187.37-fold (FIG. 11F). The data showed that the populations of B cells, T cells, monocytes, and granulocytes were reduced in both OIR retinas and myeloid SOCS3 deficient OIR retinas compared to normal retinas at P13 (FIG. 11F). To further investigate whether the function of these microglia and macrophages are altered during OIR, Gene Ontology Biological Process (GOBP) analysis was performed (FIGS. 12A-12B). Pathways involved in macrophage differentiation and phagocytosis were more robustly activated in OIR retinas and the OIR retinas lacking myeloid SOCS3 compared to normal retinas, particularly in macrophage 2, microglia 1, 2, and 3 (FIG. 12C). These microglia and macrophages are very likely associated with increased NV in OIR retinas, particularly microglia 2, 3, and macrophage 2, which were all highly increased and activated in the OIR retina lacking myeloid SOCS3 at P13, which were named as NVAMs.

RNA Velocity analysis was performed on scRNAs-seq data and projected on ForceAtlas 2 (FA) embedding (FIG. 12D) to analyze developmental lineages and cellular dynamics based on the relative abundance of nascent (unspliced) and mature (spliced) mRNA52. The direction of cell state transition and RNA velocities (indicated as arrows) showed that a differentiating trajectory from microglia 1 (root microglia) to macrophage 2 through bridge states (microglia 2 and 4) in OIR retinas and the OIR retinas lacking myeloid SOCS3. These data suggest that macrophage 2 is the most activated subset of “macrophage-like” microglia, particularly in the OIR mice lacking myeloid SOCS3. In addition, pseudotime analysis projected on ForceAtlas 2 plots (FIG. 12E) that root microglia (microglia 1) in OIR retinas is more conservative, progenitor-like, and the same cluster in the OIR retinas lacking myeloid SOCS3 included some transcriptionally different cells, closer to macrophage-like state. “Macrophage-like microglia” have longer pseudotime in OIR retinas and the OIR retinas lacking myeloid SOCS3, which suggests that it is more different from the microglia root cluster (microglia 1) than other cells. The population of macrophage 1 is similar to the root microglia in OIR, but it is much closer to the macrophage-like state in OIR retinas lacking myeloid SOCS3. Altogether, NVAMs may play important roles in retinal NV and contribute to the NV development via SOCS3.

### Control of SPP1 in NVAMs in OIR and CNV Models

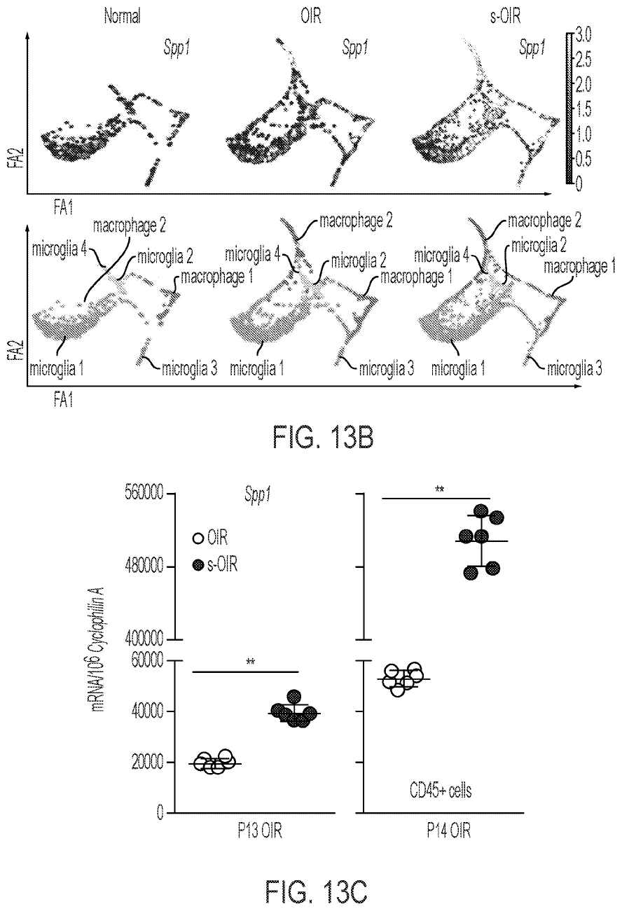

To investigate the molecular mechanism of how NVAMs contribute to the development of NV, differentially expressed genes (DEGs) in OIR retinas and myeloid SOCS3 deficient OIR retinas were analyzed using scRNA-seq data (FIG. 13A). Among the top DEGs, Spp1 was ranked as the most highly upregulated DEG in NVAMs including microglia 1, 2, 3, and macrophage 2, indicating the potential role of Spp1 in controlling the contribution of NVAMs in NV development. Spp1-expressing cells were mainly increased in NVAMs in OIR retinas, particularly the OIR retinas lacking myeloid SOCS3 showed by tSNE plots (FIG. 6A) and ForceAtlas 2 plots (FIG. 13B). The location of SPP1 expression in OIR retinas was examined on retinal cross sections (FIG. 6E). SPP1 was robustly induced in IBA1⁺ microglia and macrophages in the OIR retinas compared to weak to no detectable expression in normal retinas. SPP1 expression was further increased in the OIR retinas lacking myeloid SOCS3 at P13, and the increase was further elevated at P14. Importantly, SPP1 expression localized in IBA1⁺ cells with round shapes consistent with NVAMs (FIG. 6E, white box). The mRNA level of Spp1 in CD45⁺ cells isolated from retinas using magnetic-activated cell sorting (MACS) with CD45 antibody was significantly higher in the OIR retina lacking myeloid SOCS3 at P13 and was further increased at P14 (FIG. 13C).

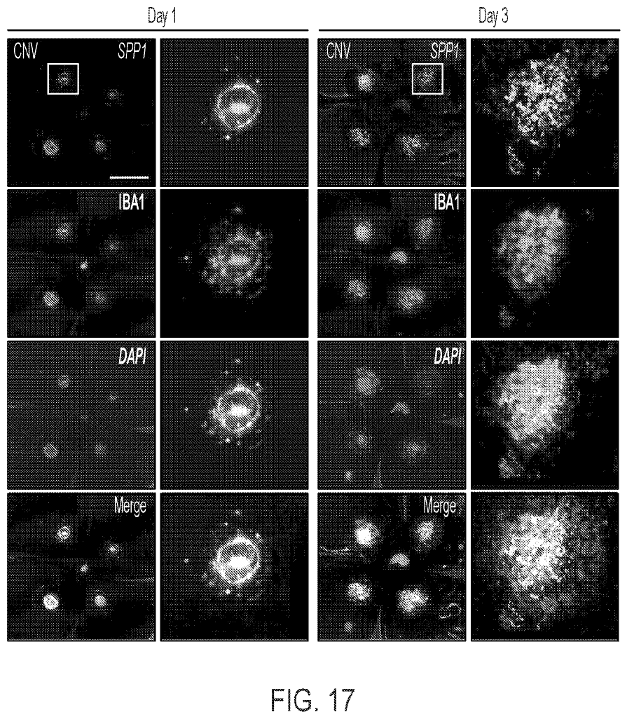

It has previously been reported that myeloid SOCS3 deficiency highly increased laser-induced CNV⁴⁹. Therefore, the expression of SPP1 in a laser-induced CNV mouse model was examined. SPP1 expression was induced at day 1 post laser exposure and continued to increase at day 3 post laser exposure in a laser-induced CNV model (FIG. 17). SPP1 expression was highly induced in IBA1⁺ cells around the CNV lesion, particularly in myeloid SOCS3 deficient mice with the larger CNV lesions (s-CNV) at day 3 (FIG. 13D). These findings suggest that SPP1-expressing NVAMs contribute to both OIR NV and laser-induced CNV development, particularly in the OIR mice lacking myeloid SOCS3 and in the CNV mice lacking myeloid SOCS3.

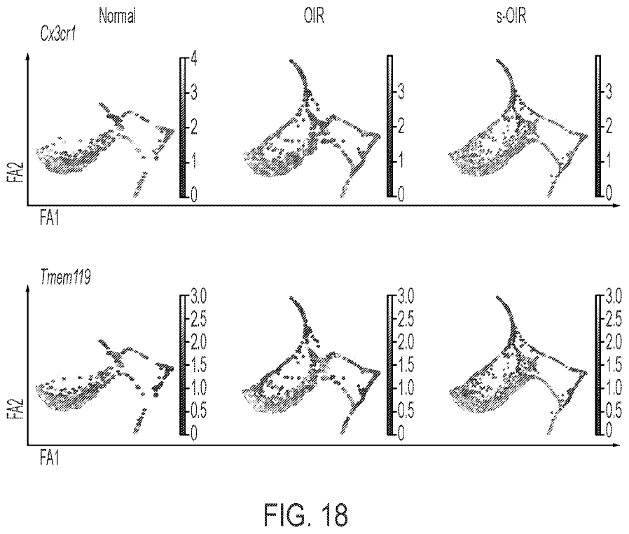

In addition, during the NVAM transition from microglia 1 to macrophage 2 (FIG. 12E), the expression of microglia markers, such as Cx3cr1 and Tmem119 was decreased (FIG. 18), and Spp1 expression was increased (FIG. 13B) suggesting a role of SPP1 during the transition of NVAM. Thus, these results indicate that SPP1 is induced in eyes with OIR or CNV and may be important in controlling pathological NV and CNV formation.

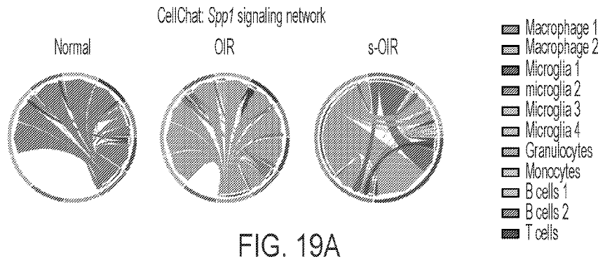

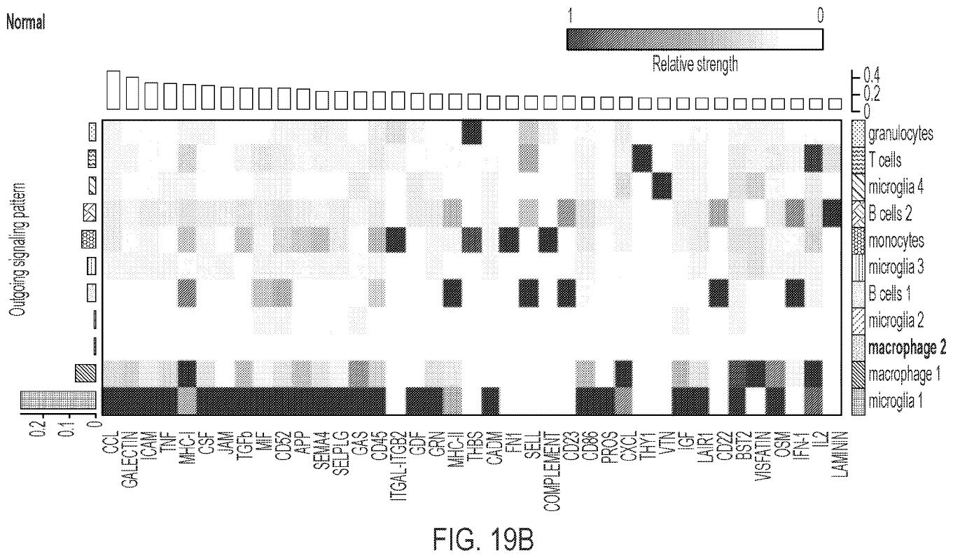

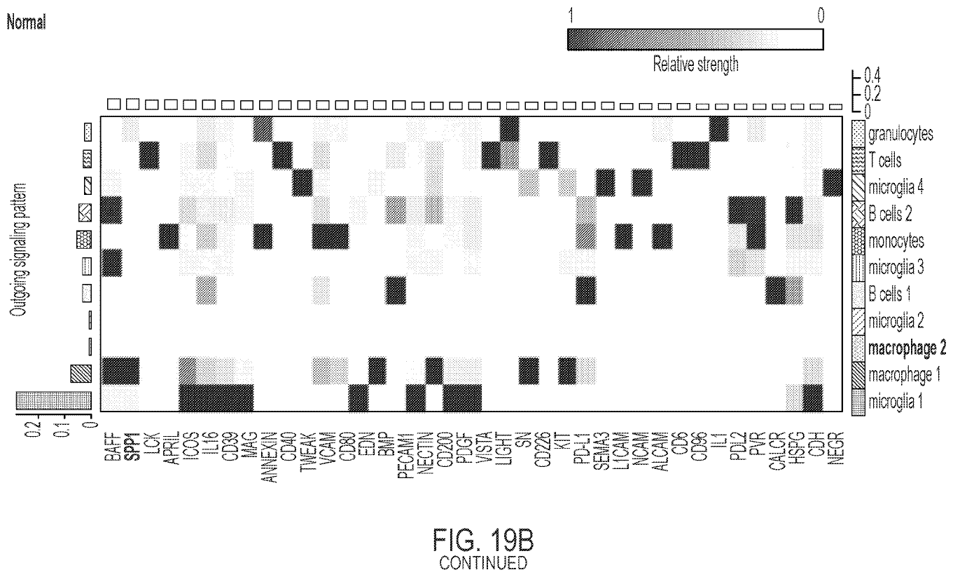

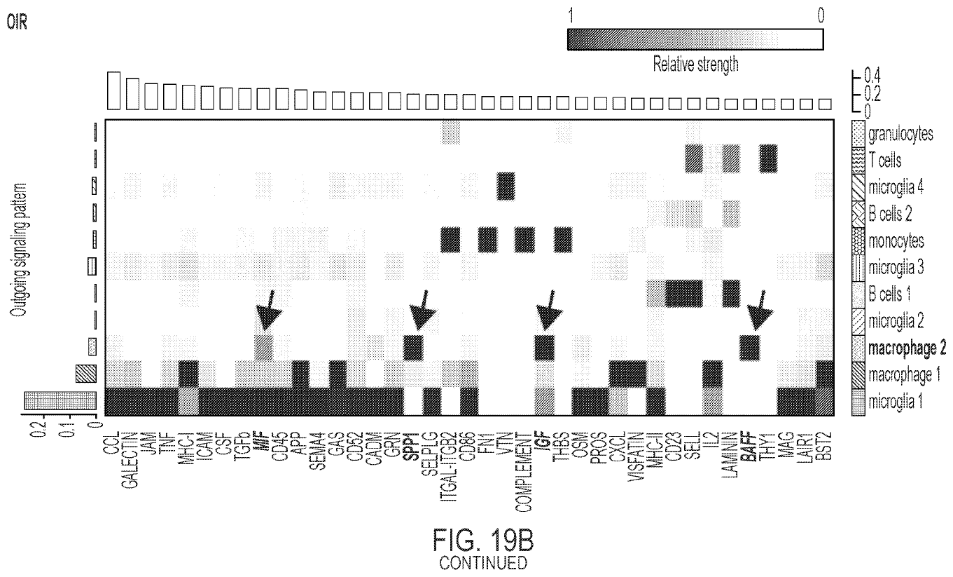

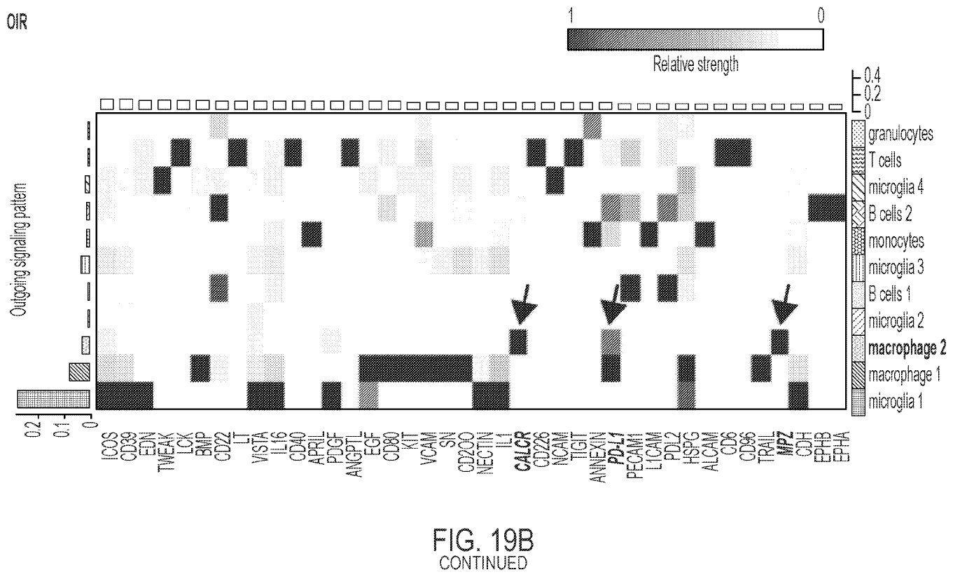

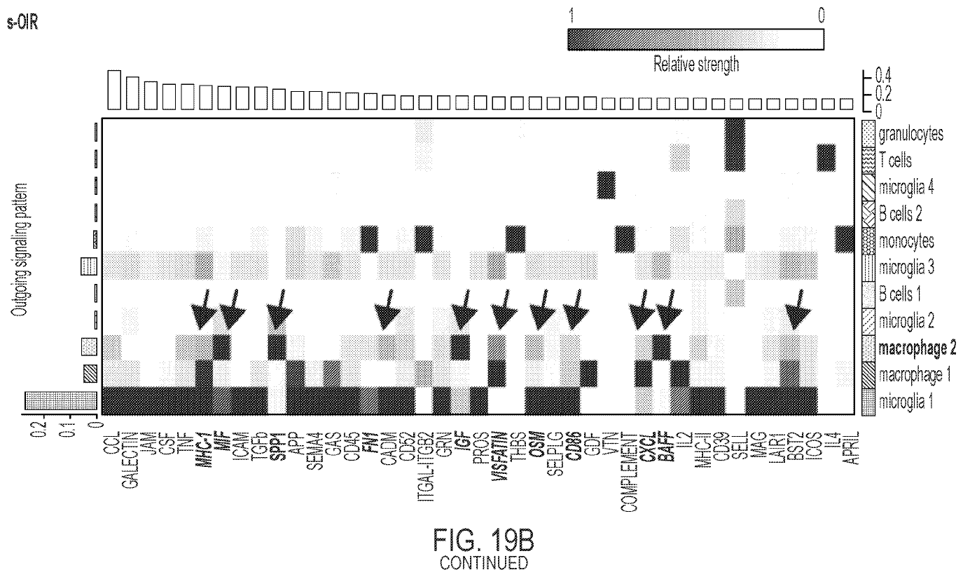

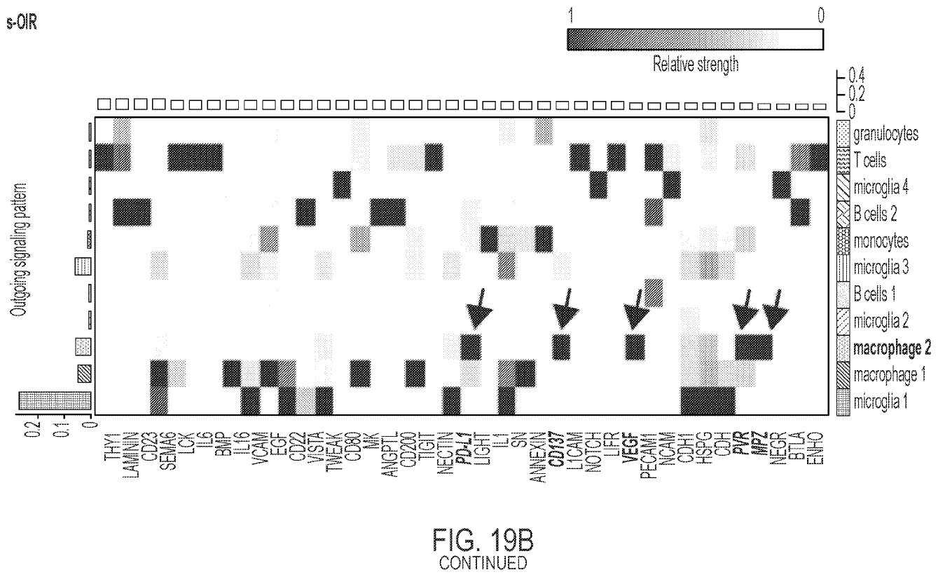

The present disclosure used CellChat analysis⁵³ to quantitatively measure the SPP1 network (FIGS. 19A-19B) and predict key incoming and outgoing signals of SPP1 to specific immune cell types and facilitate the interpretation of the intercellular communication of SPP1. Under normal conditions, SPP1 is mainly released from macrophage 1, microglia 1, and granulocytes, but during OIR, especially in the OIR mice lacking myeloid SOCS3, macrophage 2 became the most prominent source of SPP1, which is a cell type that was mainly found in OIR retinas but was only very scarcely present under normal conditions (FIGS. 11E-11F and 12C). These findings are consistent with the key role of SPP1 during NV development. CellChat analysis for outgoing signaling patterns also predicted top CellChat pathways in NVAMs (arrows in FIG. 19B). The pathways from NVAMs were activated in OIR retina and the OIR retinas lacking myeloid SOCS3, particularly in macrophage 2 mainly found in OIR retinas and OIR retinas lacking myeloid SOCS3. The top pathways in OIR were SPP1, MIF (macrophage Migration Inhibitory Factor), IGF (Insulin-like Growth Factor), BAFF (B cell Activating Factor), PD-L1 (Programmed Death Ligand 1), and MPZ (Myelin Protein Zero). There were additional activated pathways in the OIR mice lacking myeloid SOCS3: MHC-I (Major Histocompatibility Complex I), FN1 (Fibronectin 1), OSM (Oncostatin M), CD68, CXCL, CD137, VEGF, and PVR/CD155. SOCS3 knockout induced changes to these pathways in NVAMs (besides SPP1) need to be further validated.

### Socs3/STAT3 Directly Regulation of Spp1 Expression

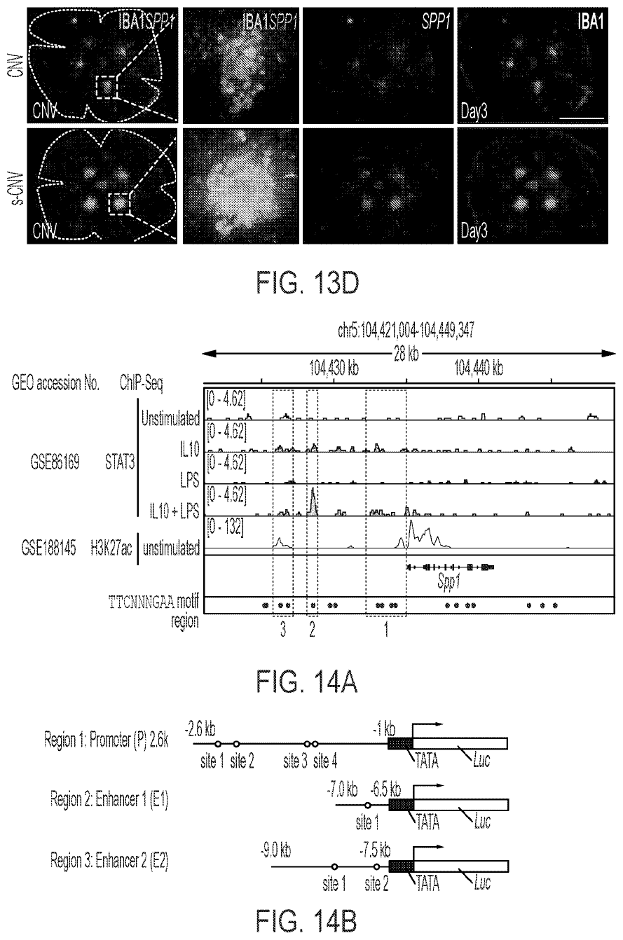

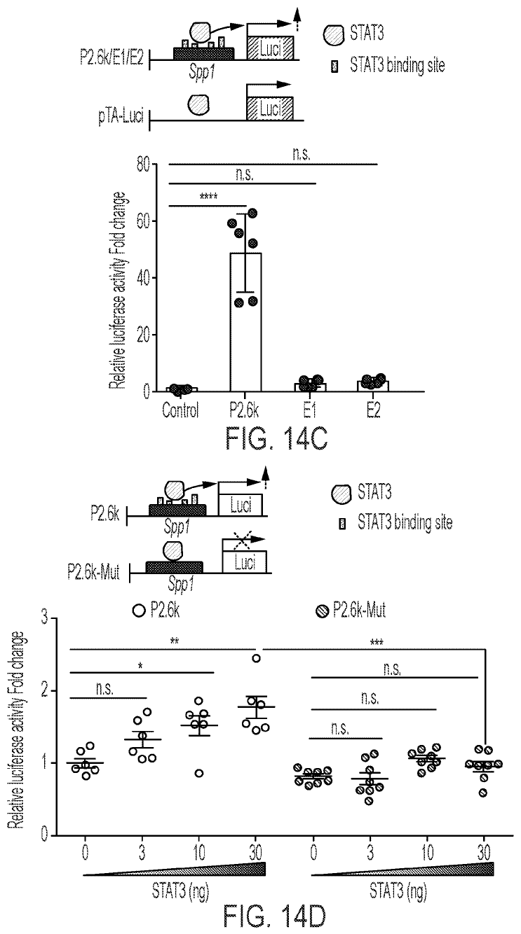

Since myeloid SOCS3 deficiency induced NV and increased Spp1^{high}-NVAMs in the myeloid SOCS3 knockout mice with OIR or laser-induced CNV, we examined whether SOCS3 can regulate Spp1 expression through the transcription factor, STAT3. To locate the potential binding sites of STAT3 on Spp1 promoter and enhancer regions, the present disclosure examined the Gene Expression Omnibus (GEO) database of STAT3 ChIP-sequencing (ChIP-seq) data in murine bone marrow-derived macrophage (BMDM) cells stimulated with IL-10 and LPS (GSE86169)⁵⁴ and the GEO database of enhancer maker H3K27AC ChIP-seq data in BMDM cells with no stimulation (GSE188145)⁵⁵ for the Spp1 gene through the Integrative Genomics Viewer. Three potential regions were identified and used for constructing luciferase reporters. The dots showed the location of the STAT3 consensus binding motif identified as TTCNNNGAA54.55 (FIG. 14A). Three luciferase reporters were generated to include the three regions: promoter (−1 kb˜−2.6 kb containing 4 potential binding motifs) (P2.6k), Enhancer 1 (−6.5 kb˜−7.0 kb containing 1 potential binding motif) (E1), and Enhancer 2 (−7.5 kb˜−9.0 kb containing 2 potential binding motif) (E2) (FIG. 14B). Dual-Luciferase reporter assay performed with these reporters showed that P2.6k promoter of Spp1 was highly active but not enhancers E1 and E2 of Spp1, suggesting the active binding potential of STAT3 on the promoter of Spp1 (FIG. 14C). To confirm the binding of STAT3 in the promoter region of Spp1, different doses of STAT3 were added into the Dual-Luciferase reporter assay and the reporter activity was increased in a STAT3 dose-dependent manner (FIG. 14D). Furthermore, the 4 potential binding motifs were mutated and the STAT3 dose-dependent manner of luciferase reporter activity for P2.6k reporter was attenuated (FIG. 14D) suggesting that Spp1 was the direct target of STAT3, which in turn was regulated by SOCS3.

### Inhibition of Myeloid SOCS3-Induced NV by SPP1 Deficiency

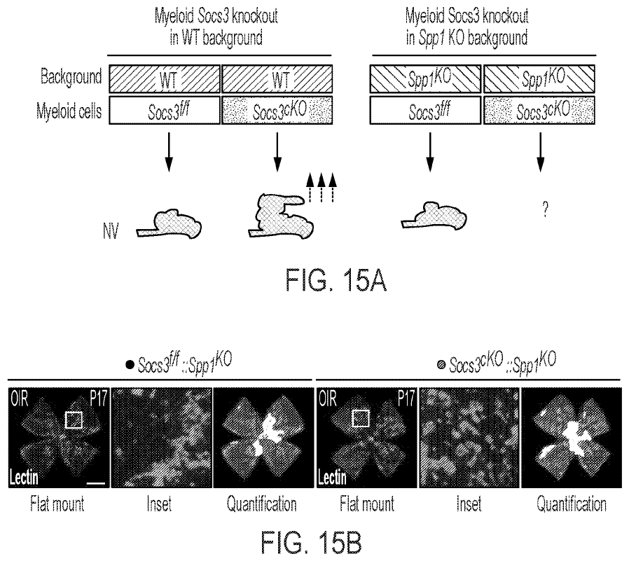

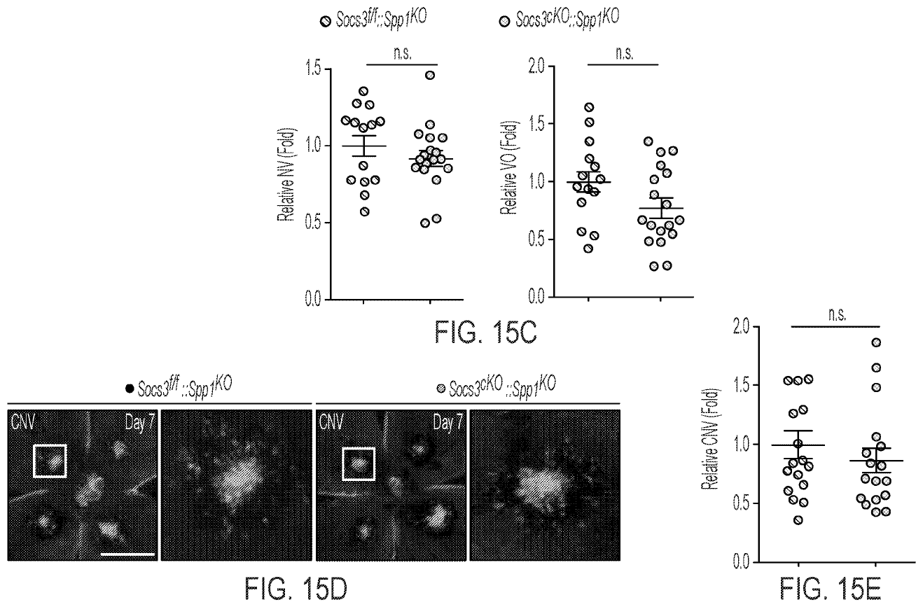

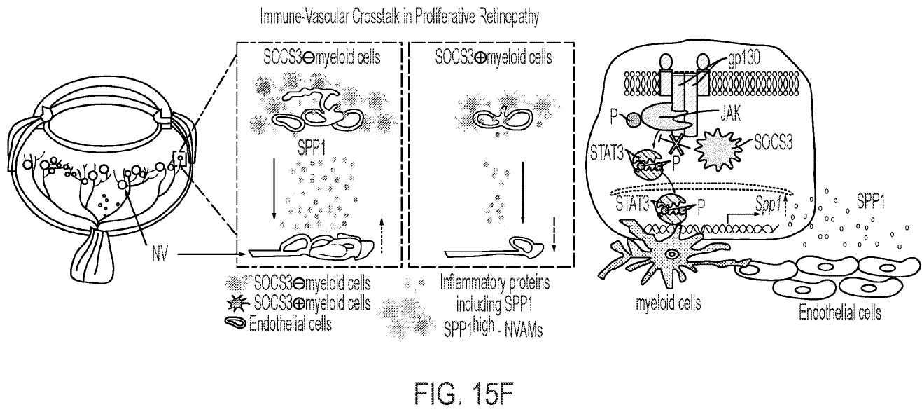

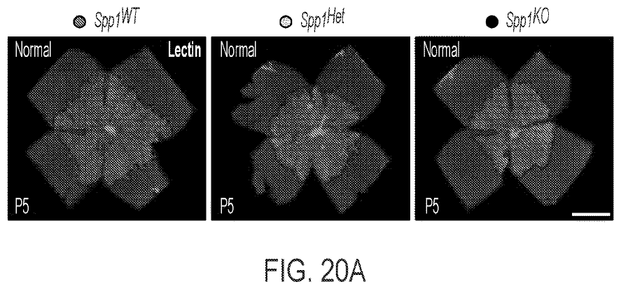

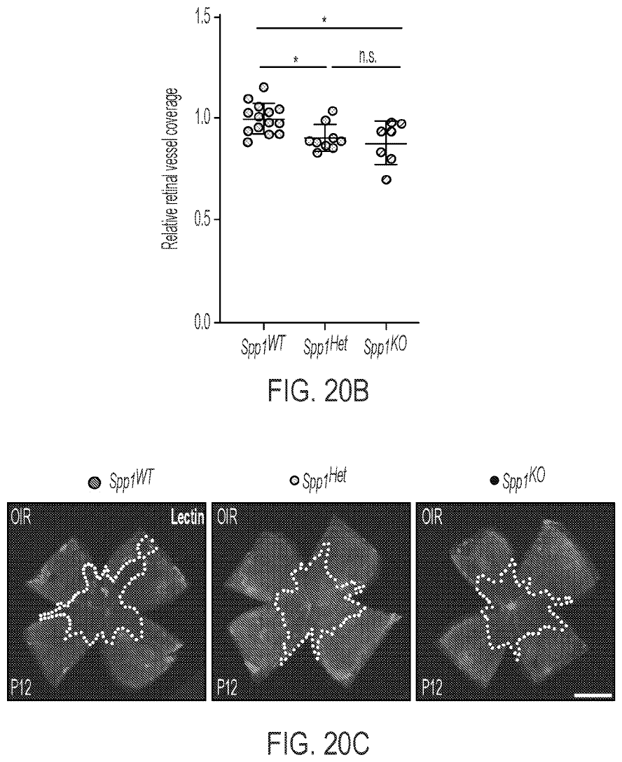

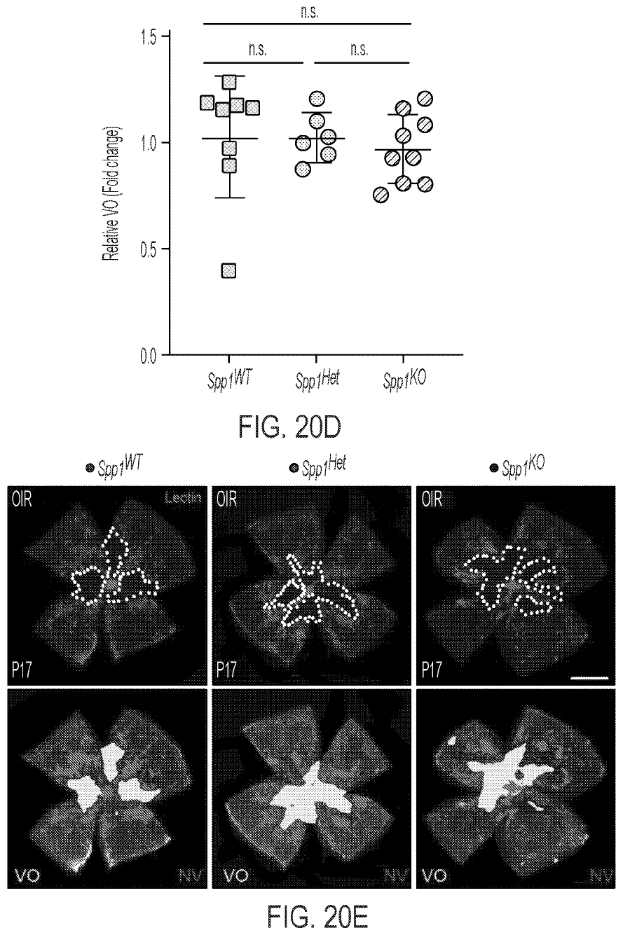

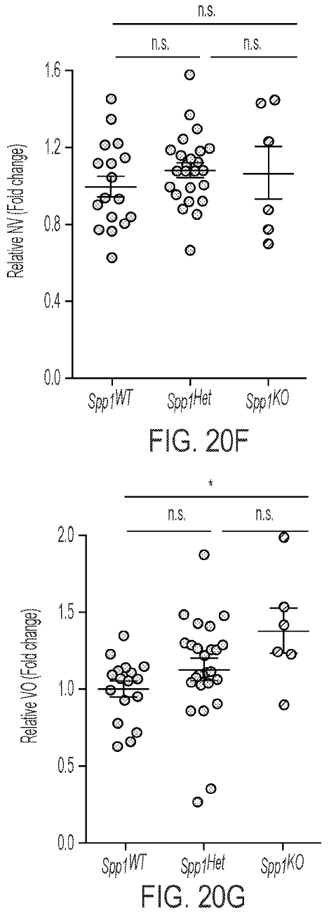

To investigate whether myeloid SOCS3-induced NV is controlled via Spp1-expressing NVAMs, Socs3 cKO were generated in Spp1 knockout (Spp1^{KO}) background (Socs3^{cKO}::Spp1^{KO}) and compared to Socs3 f/f in Spp1^{KO} background (Socs3^{f/f}::Spp1^{KO}). There was no significant difference in NV between Spp1^{KO} and Spp1^{wt} in OIR retinas at P17 (FIGS. 20A-20G). Since loss of myeloid SOCS3 in a wild type background significantly increased NV in OIR (FIGS. 4B-4G and 4K), the present disclosure assessed whether loss of myeloid SOCS3 in Spp1^{KO} background (FIG. 15A) can attenuate the increased NV. The results showed that Spp1 knockout abolished myeloid SOCS3-induced NV in Socs3^{cKO}::Spp1^{KO} OIR retinas, and NV returned to levels in line with littermate controls (Socs3^{f/f}/::Spp1^{KO}) (FIGS. 15B-15C). In addition, it has been reported that myeloid SOCS3 deficiency increased laser-induced CNV⁴⁹. The attenuation of laser-induced CNV in myeloid SOCS3 deficient mice in Spp1^{KO} background was observed (FIGS. 15D-15E). These results confirmed a critical role of the SOCS3/SPP1 axis in NV development. Taken together, these data suggest that NVAMs play an important role in controlling pathological angiogenesis through myeloid SOCS3/SPP1 signaling (FIG. 15F).

### Suppression of NV in OIR by Targeting the SOCS3/SPP1 Pathway

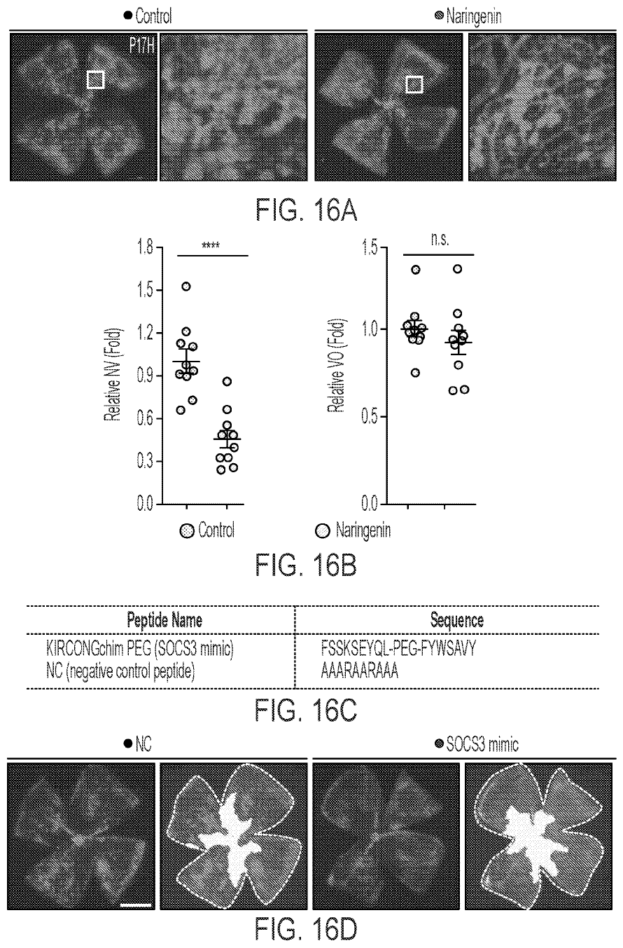

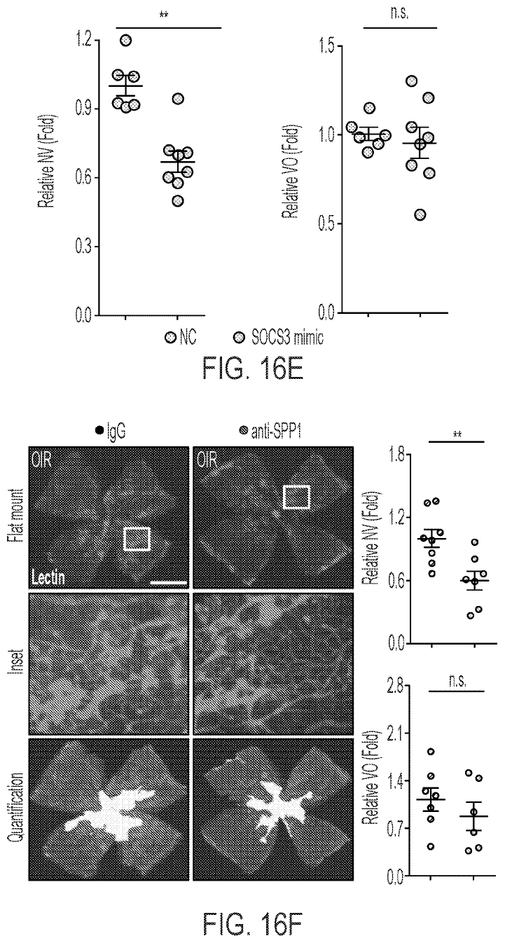

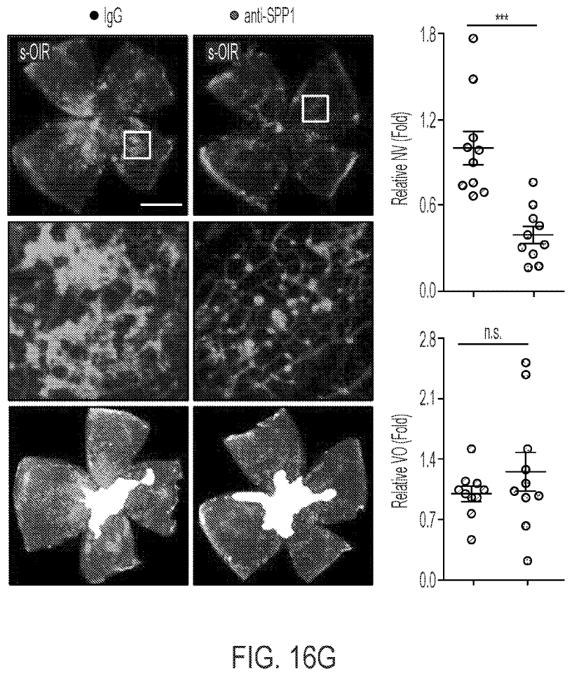

It has been reported that naringenin, a SOCS3 agonist^{56,57}, induces SOCS3 expression and significantly suppresses CNV compared to control treatment⁴⁹. Here naringenin treatment from P12 to P16 significantly reduced NV by about 50% at P17 compared to controls (FIGS. 16A-16B). The present disclosure also examined the effects of a new SOCS3 mimetic peptide, KIRCONG chim PEG (FIGS. 16C-16E). The PEGylation of the KIRCONG chim peptide aims to enhance the therapeutic properties of the mimetic by increasing solubility, reducing renal clearance, and minimizing host immune response in the host58. The data showed that KIRCONG chim PEG treatment from P12 to P16 significantly suppressed NV at P17 by about 40% (FIGS. 16D-16E). In addition, neonatal WT OIR mice were intravitreally injected as previously described⁴⁵ with a SPP1 neutralizing antibody or control antibody (IgG) at P14. Spp1 antibody treatment reduced NV by 41% in OIR (FIG. 16F) and 67% in myeloid SOCS3 deficient OIR mice (FIG. 16G) at P17 compared to control IgG treatment.

### Discussion

The present disclosure identified an important role for NV-associated myeloid cells in the development of pathological neovascularization via SOCS3/STAT3/SPP1 pathway in the OIR model, a mouse model of neovascular ROP. This study first demonstrated the accumulation of myeloid lineage cells in NV formation in the ischemic retina via SOCS3 by using myeloid-specific reporter mice with two-photon and confocal imaging techniques. Second, the study identified NV-associated myeloid lineage cells, particularly NVAMs in OIR retina using scRNA-seq technique and demonstrated the NVAM status transition during NV. Third, the study identified a key pathway, SOCS3/STAT3/SPP1, that controls NVAMs during the development of NV in OIR. Last, the study examined the therapeutic potential of targeting the SOCS3/SPP1 pathway using either SOCS3 mimic peptide, SOCS3 agonist (naringenin), or SPP1 inhibitor (neutralizing SPP1 antibody) for ocular NV treatment.

Microglia are the primary local retinal immune cells^{35,36} and are strongly associated with retinal angiogenesis^{25,30}. Recently, Liu's group performed scRNA-seq on CD11b⁺-myeloid lineage cells in OIR retinas and normal controls and identified a subpopulation of microglia that highly expressed necroptosis-related genes Rip3 and Mlkl. They provided evidence for targeting the microglia necroptosis axis as a therapy for retinal neovascular diseases including ROP⁵⁹. Huo's group performed scRNA-seq on CD11b⁺F4/80⁺ microglia/monocytes/macrophages to map these cells in retinas with or without OIR and found several unique types of microglia associated with proliferative retinopathy, such as microglia subsets with high self-renewal capacity and a hyper-metabolic microglia subset⁶⁰. These findings support the critical roles of microglia in the development of ROP. However, not all ROP causes blindness. Some ROP infants are mild and can recover without treatment, but others with severe ROP need treatment to prevent blindness. Understanding the mechanisms underlying the development of severe ROP is essential for improving patient outcomes for these infants. Xu's group reported that loss of myeloid SOCS3 enhances NV in OIR46. The data also showed that depletion of myeloid Socs3 (Socs3 cKO) significantly increased retinal NV and worsened tortuosity of posterior retinal vessels in OIR retinas. Retinal vascular tortuosity is associated with severe form of ROP⁵⁰, and is a feature of ROP plus disease, which is characterized by the most severe vascular changes of dilation and tortuosity⁶¹. Socs3 cKO OIR mice were used as an OIR of a more advanced severe ROP to investigate the role of myeloid lineage cells during severe ROP pathogenesis. The scRNA-seq data analysis from retinas with or without OIR showed that the relative abundance of several subpopulations of microglia (microglia 1, 2, 3) and macrophage 2 were increased in OIR retinas. These populations of cells were further increased in the OIR retinas lacking myeloid SOCS3, modeling more advanced severe ROP. This is consistent with the increased NV and the activation of pathways in macrophage differentiation and phagocytosis. Accordingly, these populations of cells were associated with increased NV and were labeled as NVAMs. Recently an important subset of disease-associated microglia (DAM) was identified at sites of brain lesions in Alzheimer's disease (AD), amyotrophic lateral sclerosis, and aging^{62, 63}. The gene signature in these newly identified clusters of NVAMs is very similar to that of reported DAMs in brain with AD^{62, 63}, which is characterized by immune cells expressing typical microglial markers (Iba1, Cst3, and Hexb), coincident with downregulation of “homeostatic” microglia genes (P2ry12, Cx3cr1, and Teme119), and upregulation of genes involved in lysosomal phagocytic and lipid metabolism pathways (Apoe, Lp1, and Trem2).

The present disclosure demonstrated that there was no statistical difference in NV in Spp1 knockout (Spp1^{KO} OIR) retinas compared to NV in wild type OIR retinas and significantly increased NV in OIR mice lacking myeloid SOCS3 compared to NV in Socs3^{f/f} OIR. However, the significantly increased NV was attenuated in Spp1^{KO}Socs3^{cKO} double knockout mice with OIR. This is in agreement with slightly increased level of Spp1 in OIR and highly increased levels in the OIR retinas lacking myeloid SOCS3 compared to normal retina in FIG. 13A. These data support the conclusion that Spp1 controls ROP development, particularly in severe ROP.

SPP1 is expressed by multiple other cell types in the mouse retinas under different physiological and pathological conditions, such as alpha retinal ganglion cells⁶⁴ and optic nerve astrocytes after injury⁶⁵. To rule out the contribution of Spp1 from other cell types to severe NV development in the OIR retina lacking myeloid SOCS3, NV in OIR retinas or OIR retinas lacking myeloid SOCS3 were compared to the same genetic background with similar levels of SPP1. In the Spp1^{KO} background, this study compared NV between OIR mice lacking myeloid SOCS3 to control OIR mice (Socs3 flox control). Increased NV caused by myeloid SOCS3 deficiency in a wild-type background was not seen in OIR retinas lacking myeloid SOCS3 in a Spp1 KO background. Thus, significantly increased NV in OIR mice lacking myeloid SOCS3 was controlled by Spp1. Alternatively. Spp1 flox mice⁶⁵ can be used to specifically knockout Spp1 in myeloid cells by breeding them with Socs3 cKO driven by LysM-Cre mice.

SOCS3/SPP1 could control NV in OIR retinas indirectly by regulating microglia/macrophage function to modulate other NVMAs-expressing proangiogenic factors, such as the VEGF pathway, or directly control of endothelial cell growth as previously reported^{66, 67} by activating endothelial Kit/Akt/mTOR pathway⁶⁷.

Multiple transcriptional factors that bind to the SPP1 locus have been identified in different disease contexts. It has been reported that c-Myc and OCT-1 bind to the proximal promoter of Spp1 gene in U251MG and U87MG human glioma cells⁶⁸. Transcription factors ETS-1 and RUNX2 regulate Spp1 expression in colorectal cancer cells⁶⁹. In melanoma cells transcription factors c-Myb⁷⁰, AML-1a, and C/EBPa bind to the Spp1 gene promoter⁷¹. The present disclosure identified a direct transcriptional control of Spp1 by STAT3 in retinas of OIR mice lacking myeloid SOCS3. However, whether Spp1 is synergistically controlled by other transcriptional factors in these mice, such as HIFIa72, needs to be further explored.

The present disclosure demonstrates the important translational potential of targeting the SOCS3/SPP1 pathway to treat vascular eye diseases including AMD and ROP. It was previously reported that naringenin, a SOCS3 agonist^{56,57}, significantly suppresses laser-induced CNV⁴⁹ in a mouse model of neovascular AMD. This study demonstrated that naringenin reduced retinal NV by about 50% compared to control treatment in OIR. It was previously reported that a SOCS3 mimic peptide, KIRESS peptide (KIRESS) reduced the laser-induced CNV by 24% compared to control treatment⁴⁹. This study analyzed the effects of a new chimeric water-soluble SOCS3 mimetic, KIRCONG chim PEG, in the OIR mouse model. An initial compound, KIRESS, includes the KIR and ESS regions of SOCS3, which are bound to JAK2, and activates STAT3 levels both in athymic nude mice bearing SCC xenografts⁷³ and in triple negative breast cancer mouse models to reduce tumor growth⁷⁴. Another SOCS3 mimetic, KIRCONG chim, a chimeric peptidomimetic with two non-contiguous protein fragments (KIR and CONG) linked by non-native β-Alanine molecules, is also able to mimic SOCS3 biology in vascular smooth muscle cells⁷⁵, but has a limited water-solubility. The present disclosure analyzed the effect of a new analogue of SOCS3, KIRCONG chim PEG compound⁷⁶ in OIR. KIRCONG chim PEG contains a polyethylene glycol (PEG) moiety as a spacer instead of the two B-Alanine molecules of KIRCONG chim. KIRCONG chim PEG treatment from P12 to P16 suppressed NV at P17 by about 40% without influencing VO. Additionally, the data showed that SPP1 neutralizing antibodies delivered by intravitreal injection prevented NV in OIR mice lacking myeloid SOCS3, demonstrating the translational value of targeting SPP1 for severe ROP. Beguier et al reported that SPP1 was robustly induced by high-temperature requirement A serine peptidase 1 (HTRA1) which is strongly increased in AMD retinas and HTRA1-induced increase in CNV formation was significantly reduced by SPP1 neutralizing antibody⁷⁷ suggesting the therapeutic potential of SPP1 inhibitors for the treatment of AMD. The NV in two models (OIR and CNV) were affected by varying degrees in Spp1^{KO} mice or anti-SPP1 treatment^{48, 77, 78} under different contexts. Systemic application and local intravitreal injection of anti-SPP1 may also function differently on NV development⁴⁸. The underlying mechanisms need to be explored.

These findings provide the first proof of concept that NVAMs determine pathological retinal blood vessel growth in vascular eye diseases and demonstrate that an important pathway, the SOCS3/STAT3/SPP1 pathway, controls NVAMs during the development of pathological ocular NV. Targeting NVAMs via the SOCS3/STAT3/SPP1 pathway may thus serve as a completely new direction to potentially treat ocular neovascularization in vascular eye diseases and may provide evidence to treat other inflammation-mediated vascular diseases for broad therapeutic value.

### Materials and Methods

### Study Design

The objective of this study was to investigate the population of immune cells, specifically myeloid lineage cells, that contribute to ocular NV development and investigate the underlying molecular mechanisms of immune-vascular crosstalk in vascular eye diseases, such as neovascular AMD and ROP. First, an NV mouse model was generated using myeloid-specific reporter mice labeled with fluorescent proteins to visualize myeloid lineage cells during NV development with confocal imaging and two-photon microscopy. The study examined the role of a key immune regulator, SOCS3, in controlling NV by generating loss-of-function and gain-of function of myeloid SOCS3 mice with OIR. Next, scRNA-seq was performed on CD45⁺ immune cells isolated from normal retinas, OIR retinas, and SOCS3 deficient OIR retinas to analyze the changes of immune cell distribution during NV. To identify the cell types that contribute to NV formation and the underlying molecular changes, single-cell transcriptomic data analysis was performed and DEG, GOBP, RNA Velocity, and CellChat analysis for scRNA-seq data were also performed. The top ranked DEG expression from scRNA-seq data analysis was confirmed using qPCR and immunostaining on mouse cross sections and retinal flat mounts from normal mice, OIR mice, and OIR mice lacking myeloid SOCS3. The direct regulation of SPP1, a downstream gene of SOCS3/STAT3, was further validated using luciferase reporter assay. The role of SPP1 in myeloid SOCS3 ablation-induced NV formation was confirmed using Spp1 and myeloid Socs3 double knockout mice. The conclusion was further confirmed in a second NV mouse model, a laser-induced CNV model mimicking some features of neovascular AMD. Lastly, the study tested the potential therapeutic value of targeting the SOCS3/SPP1 pathway by assessing the effects of SOCS3 agonists and SPP1 neutralizing antibodies during NV formation. All mouse studies were approved by Boston Children's Hospital Institutional Animal Care and Use Committee. The ethical use of animals was according to the guidance of The Association for Research in Vision and Ophthalmology. Male and female mice were all included for experiments and randomly grouped. Age-matched flox/flox mice, Cre mice, or wild type mice were used as controls. The images were quantified by masked researchers. All experiments were repeated independently at least three times. The sample size for each experiment was included in the Brief Description of the Figures.

### Animals

A floxed Socs3 (Socs3 f/f) mouse line is a gift from Dr. A. Yoshimura⁷⁹. A floxed mouse line for Socs3 overexpression (Socs3OE) is a gift from Dr. Allison W. Xu⁸⁰. Cre^{LysM} mice (stock #004781), C57BL/6J mice (stock #000664), mTmG reporter mice (stock #007576), Ai9 reporter mice (stock #007909), and Spp1 knockout mice (stock #004936) were obtained from the Jackson Laboratory. Cre^{LysM} mice were used to generate myeloid-specific Socs3 knockout mice (Socs3 cKO) and myeloid-specific Socs3 overexpression mice (Socs3 cOE). The flox/flox mice and Cre^{LysM} mice were used as controls.

### Oxygen-Induced Retinopathy (OIR) Model

OIR was carried out using neonatal mice as described previously⁸¹. Briefly, mouse pups with nursing female mice were exposed to 75% oxygen from postnatal day (P) 7 to 12, then returned to room air until P17 for phenotypical analysis and gene expression analysis at indicated times. For phenotypical analysis, the retinas were collected at P17, dissected, and stained with fluorescent dye conjugated with Griffonia Simplicifolia Isolectin IB4 (Invitrogen) overnight and flat-mounted. The avascular (vaso-obliteration, VO) and pathological NV areas were quantified⁸² by masked researchers using Image J (National Institutes of Health) and fully automated deep learning segmentation method⁸³. The exclusion criteria for the OIR mouse model were used as previously described⁸⁴.

### Immunostaining, Confocal Imaging, and Two-Photo Microscopy Imaging

Mouse eyes were isolated and fixed with 4% paraformaldehyde (PFA) at room temperature for 1 hour. The fixed eyes were washed with 0.01 M phosphate buffered saline

(PBS) for three times. Retinas were dissected from the fixed eyes, embedded, sectioned, and permeabilized by 0.01 M PBS with 0.3% Triton X-100. The flat-mounted retinas or cross sections were stained with antibodies according to the manufacturer instructions and imaged using a confocal microscope (Zeiss 700). For two-photon imaging, mTmG reporter mice were intravenously injected with Qtracker655/CD31 antibody before being euthanized; retinas were dissected without fixation and mounted for imaging. The primary antibodies used were: IBA1 (Wako, 019-19741, RRID: AB_839504), Isolectin B4 (ThermoFisher Scientific, 121411, RRID: AB_23146), CD31 (MEC 13.3, BD Biosciences, 550274, RRID: AB_393571), SOCS3 (abcam, ab16030, RRID: AB_443287), PE anti-mouse/human CD11b (BioLegend, 101207, RRID: AB_312790), GFP (Abcam, ab13970, RRID: AB_300798), and the secondary antibodies used were: Goat anti-Rabbit IgG, Alexa Fluor™ 594 (Thermofisher, A-11037, RRID: AB_2534095), Goat anti-Rabbit IgG, Alexa Fluor™ 488 (Thermofisher, A-11008, RRID: AB_143165), Goat anti-Chicken IgY, FITC (Thermofisher, A16055, RRID: AB_2534728), Goat anti-Mouse IgG, IgM, IgA (H+L), Alexa Fluor™ 488 (Thermofisher, A-10667, RRID: AB_2534057), Goat anti-Mouse IgG, Alexa Fluor™ 594 (Thermofisher, A-11005, RRID: AB_141372), Donkey anti-Rabbit, Alexa Fluor™ 647 (Thermofisher, A-31573, RRID: AB_2536183), and Donkey anti-Goat IgG, Alexa Fluor™ 647 (Thermofisher, A-21447, RRID: AB_141844), and DAPI (Vector Labs, H-1200-10).

### Mouse Fundus Imaging

Mice were anesthetized by intraperitoneal injection of a mixture of ketamine (10 mg/kg) and xylazine (6 mg/kg). Tropicamide solution (1%) (Akorn, NDC 17478-102-12) was added to the eyes of anesthetized mice to dilate the pupils. Eyes were flushed with artificial tears (K.C. Pharmaceuticals, NDC 55651-03) to remove tropicamide before imaging. Fluorescein sodium salt (Sigma, F6377) was injected into the intraperitoneal cavity. The eyes of the anesthetized mouse were positioned for fundus imaging using the Retinal Imaging System (Micron IV, Phoenix). The eyes were kept moist with artificial tears during imaging and the animal were placed on warming pads after imaging until they returned to consciousness. Images were captured and saved by Retinal Imaging System (Micron IV, Phoenix).

### Fluorescence-Activated Cell Sorting (FACS)

Fresh retinas were isolated from mouse eyes and the single cell suspensions were generated by Papain Dissociation System (Worthington-biochem LK003150). Retina cells were incubated with FITC-CD45 antibody for 30 min on ice in a dark box and were washed 3 times by centrifugation at 1000 rpm for 3 minutes then resuspended cells in ice-cold FACS buffer containing 5% bovine serum albumin (Invitrogen, AM2616) and 0.5 mM EDTA in PBS. Cells were kept in the dark on ice for sorting. Dead cells were removed by LIVE/DEAD™ Fixable Dead Cell Stain Sampler Kit (L34960) during sorting. CD45 positive (+) cells were collected for SCRNA-seq.

Single-Cell RNA Sequencing (scRNA-Seq) and Data Analysis

scRNA-seq was performed by the Translational Immunogenomics Lab (TIGL) at Dana Farber Cancer Institute using the 10× Genomics platform. The raw data were analyzed using the 10× Cellranger pipeline. Sequencing read counts in single cells were processed and analyzed using the Seurat 4.0⁸⁵. Doublets, dead cells, and empty droplets were removed and cells with >500 and <5000 detected genes and <8% of transcripts that are of mitochondrial origin were included. Sequentially, data were normalized using the “LogNormalize” method, 2000 highly variable feature was selected, data were centered and scaled by their root mean square, and PCA analysis was performed for dimension reduction. Further, the Louvain optimization-based clustering method was used on the top 20 principal components to identify clusters, which were then assigned to cell types based on well-evidenced cell markers. CD45-negative cells were filtered and CD45⁺ cells were clustered and focused on the downstream analysis. The cell clusters were visualized in reduced dimensions of t-distributed stochastic neighbor embedding (ISNE). To compare gene expression between cell types. Wilcoxon rank sum test was used, and Bonferroni method was applied to adjust p values to control false positives due to multiple comparisons. SCANNER⁸⁶ was used for data visualization and exploration. Software and Algorithms used in this study was listed in Table 1.

### GOBP Pathway Analysis

Escape package⁸⁷ was utilized to perform GSEA pathway analysis⁸⁸. The pathways for macrophage differentiation (GOBP_MACROPHAGE_DIFFERENTIATION) and phagocytosis (GOBP_PHAGOCYTOSIS) were downloaded from Molecular Signatures Database⁸⁹, to perform the enrichment for Normal, OIR, and s-OIR datasets and visualize the results by exporting the pathways activity scores and utilizing ggplot2 geom_histogram( ) function.

### RNA Velocity

Cellranger, samtools, velocyto, and scvelo packages were utilized to perform the RNA Velocity analysis52. First, by using the cellranger count function on.fastq files in Ubuntu WSL 20.04, a Cellranger output folder was generated, and the samtools sort function was used in the samtools package (−t CB −O BAM) to create the cellsorted_possorted_genome_bam.bam file from the possorted_genome_bam.bam file. The generated file was used for making the .loom file upon utilizing the velocyto run 10×function of the velocyto package. The reference genome used for generating the .loom file is GENCODE 90 annotation v.M27, and the proper repeat annotation for the reference genome was obtained from the UCSC genome browser in .gtf format. Once the .loom file is generated, the .h5Seurat annotated objects were converted to .h5ad objects to transfer them from R to the Python environment. The sc Velo package 91 for the RNA Velocity analysis was used. The .loom files with the respective data files previously generated were merged, processed, and annotated. The following files were filtered and normalized. The first and second order moments (means and uncentered variances) were computed among nearest neighbors in PCA space with the scvelo.pp.moments( ) function. Then velocity (scvelo.tl.velocity( ) was estimated, the velocity_graph (scvelo.tl.velocity_graph( ) was built, and the velocities were projected using scvelo.pl.velocity_embedding_stream( ) and scvelo.pl.velocity_embedding_grid( ) on ForceAtlas2 (basis=‘draw_graph_fa’) embedding. Software and Algorithms was listed in Table 1.

### Cell Chat Analysis

The CellChat package⁵³ was used to obtain the information related to incoming and outgoing clusters ligand-receptors interactions. Seurat objects and the standard pipeline were used for analysis⁹². To avoid the projection reducing the dropout effects of signaling genes, in particular for possible zero expression of subunits of ligands/receptors, raw.use=FALSE setting in computeCommunProb function was used, and population.size=TRUE parameter was also used to consider the effect of cell proportion in each cell group in the probability calculation. Software and Algorithms used in this study was listed in Table 1.

### Magnetic-Activated Cell Sorting (MACS)

Fresh retinas were isolated from mouse eyes and a single-cell suspension was generated by Papain Dissociation System (Worthington Biochem, LK003150). Cells were passed through a 70 μM nylon mesh (Falcon, 352350), pelleted by centrifugation at 300×g for 10 minutes, and resuspended in degassed working solution containing PBS (pH 7.2), 0.5% bovine serum albumin (Invitrogen, AM2616), and 2 mM EDTA. CD45 microbeads (Miltenyi Biotec, 130-052-301) were incubated with the cells for 15 minutes at 4° C. Cells were washed and resuspended to a concentration of 10⁸ cells per 500 μL working solution. MS columns (Miltenyi Biotec, 130-042-201) were mounted onto the MiniMACS (Miltenyi Biotec, 130-042-102) magnetic separator and were rinsed with working solution before single-cell suspensions were loaded. Unlabeled cells were washed 3 times with 500 μL working solution and collected. MS columns were removed from the MiniMACS separator and 1 mL working solution was added to the columns. Magnetically labeled CD45⁺ cell fractions were immediately flushed out of the column with a plunger and collected.

### Total RNA Extraction and Quantitative RT-PCR

CD45⁺ immune cells isolated from retinas were used for total RNA extraction using kit (Zymo Research, R1054) as per manufacturer's instructions. Total RNA was used for cDNA synthesis using iScript™ cDNA Synthesis Kit (Bio-Rad, 1708890). The cDNA was used for quantitative real time PCR using SYBR Green (Apex Bio, K1070) with Cyclophilin A as an internal control. The PCR primer sequences are listed in Table 2.

### Laser-Induced CNV Model

Laser-induced CNV was performed as described⁹³. Briefly, 6˜8-week-old mice were anesthetized using ketamine/xylazine solution by intraperitoneal injection followed by pupil dilation with 1% tropicamide. Laser burns were induced using an image-guided laser system (Micron IV, Phoenix) in mouse eye. Mice were euthanized at the indicated time points after laser exposure and mouse eyes were fixed in 4% paraformaldehyde (Fisher Scientific, AAJ19943K2) in 10 mM PBS for 1 hour. The retinal pigment epithelium-sclera-choroid complex was dissected, permeabilized with 0.2% Triton X-100 in 10 mM PBS for 1 hour, stained with the indicated antibodies, and flat-mounted onto slides using mounting medium (Vector Labs, H-1000-10) after washing with PBS. The flat-mount images were taken using AxioObserver.Z1 microscope (Zeiss) or the Zeiss 700/710 confocal microscope. The CNV lesions were quantified using ImageJ by masked researchers. Exclusion criteria for laser-induced CNV lesions were used as described 93.

### Luciferase Reporter Constructs Preparation and Luciferase Reporter Assay.

DNA fragments containing putative STAT3 binding sites on the Spp1 promoter regions including promoter 2.6k (P2.6K), enhancer 1 (E1), and enhancer 2 (E2) shown in FIG. 14B were amplified from mouse genomic DNA with primers (Table 3) and cloned into luciferase report vector pTA-Luc (Clontech) to generate Spp1 luciferase reporter constructs. Mutant constructs of Spp1 luciferase reporter with deletion of STAT3 binding sites were constructed with mutagenesis primers for each binding site (Table 3). HEK293T cells (ATCC, CRL-3216) were transiently transfected with STAT3 expressing plasmid, wild type or mutant pTA-Luc-Spp1 luciferase reporters. Renilla luciferase vector alone was used as control. At 36 hours post-transfection, cells were lysed in passive lysis buffer in Dual-Luciferase assay system (Promega) and luciferase reporter activities were measured using an EnSight Multimode Plate Reader (Perkin Elmer luminometer).

### Socs3 Agonists and Spp1 Antibody Treatment.

SOCS3 agonist, naringenin (400 mg/kg/day, gavage) 49, and control reagents were administrated to C57BL/6J OIR mice from P12 to P16. SOCS3 peptidomimetic and negative control (NC) peptides were synthesized and purified as described 74. Reagents were obtained from Iris Biotech and Merck (Germany). Solvents were obtained from Romil (Dublin, Ireland). Reverse-Phase columns and the LC-MS system were obtained from ThermoFisher (Waltham, MA). The building block Fmoc-NH-(PEG)-COOH (9 atoms) was employed as PEG moiety. To aid cellular penetration, the cell penetrating peptide (CPP)-Tat (48-60 fragment of HIV Tat protein, GRKKRRQRRRPQGG (SEQ ID NO: 21)), carrying fluorescein isothiocyanate as fluorophore, was conjugated to SOCS3 mimetic and NC sequences. Crude products were purified through RP-HPLC, applying a linear gradient of CH3CN to water, both 0.1% TFA, from 5 to 70% over 15 min, using a 2.2×5 cm C18 column at a flow rate of 20 mL/min and a UV detector at a wavelength of 210 nm. The identity and purity of main products were evaluated by LC-MS and purified compounds were lyophilized and stored at −20° C. until use. Purified compounds were diluted into PBS to a final concentration of 10 μg/μL prior to injections. SOCS3 peptidomimetic and NC peptide were administered intraperitoneally into mice with OIR at a dose of 10 mg/kg from P13 to P16. Mouse SPP1 neutralizing antibody (R&D Systems, AF808) and goat immunoglobulin G (IgG) (R&D Systems, AB-109-C) were diluted into sterile PBS to a concentration of 1 μg/μL. One microgram of SPP1 neutralizing antibody or IgG was intravitreally injected into each eye of OIR mice at P14 as previously described (43).

### Statistics

GraphPad Prism was used for statistical analyses. Results are presented as means±Standard Error of the Mean. Mann-Whitney test was used for two-group comparison. Kruskai-Wallis Dunn's test was used for multiple-group comparison. ca test was used to determine the statistically difference of cell population between groups. P values<0.05 were considered statistically significant.

### Data and Code Availability

The scRNA-seq datasets are deposited to the Genome Expression Omnibus (GEO) under the accession numbers (GSE227861) (nebi.nlm.nih.gov/geo/query/acc.cgi?&acc=GSE227861). The code and processed annotated datasets generated in this study for reproducing the bioinformatical analysis can be found on the following GitHub repository: github.com/mcrewcow/Sun_Ye_microglia_macrophages_paper. GSE86169⁵⁴ and GSE188145⁵⁵ arc available through the original publications. All datasets generated are available from the corresponding author upon request. All other data are available in the main text or the supplementary materials.

### REFERENCES

1. Hartnett M E, Penn J S. Mechanisms and management of retinopathy of prematurity. N Engl J Med 2013; 368:1162-1163.
2. Tasman W. Patz A, McNamara J A, Kaiser R S, Trese M T, Smith B T. Retinopathy of prematurity: the life of a lifetime disease. Am J Ophthalmol 2006; 141:167-174.
3. Blencowe H, Cousens S, Oestergaard M Z et al. National, regional, and worldwide estimates of preterm birth rates in the year 2010 with time trends since 1990 for selected countries: a systematic analysis and implications. Lancet 2012; 379:2162-2172.
4. Lutty G A, Chan-Ling T, Phelps D L et al. Proceedings of the Third International Symposium on Retinopathy of Prematurity: an update on ROP from the lab to the nursery (November 2003, Anaheim, California). Molecular vision 2006; 12:532-580.
5. Tolsma K W. Allred E N, Chen M L, Duker J, Leviton A, Dammann O. Neonatal bacteremia and retinopathy of prematurity: the ELGAN study. Archives of ophthalmology 2011; 129:1555-1563.
6. Chen M, Citil A, McCabe F et al. Infection, oxygen, and immaturity: interacting risk factors for retinopathy of prematurity. Neonatology 2011; 99:125-132.
7. Polam S. Koons A, Anwar M, Shen-Schwarz S. Hegyi T. Effect of chorioamnionitis on neurodevelopmental outcome in preterm infants. Arch Pediatr Adolesc Med 2005; 159:1032-1035.
8. Moscuzza F. Belcari F. Nardini V et al. Correlation between placental histopathology and fetal/neonatal outcome: chorioamnionitis and funisitis are associated to intraventricular haemorrage and retinopathy of prematurity in preterm newborns. Gynecol Endocrinol 2011; 27:319-323.
9. Lee J. Dammann O. Perinatal infection, inflammation, and retinopathy of prematurity. Semin Fetal Neonatal Med 2012; 17:26-29.
10. Klinger G. Levy I. Sirota L et al. Outcome of early-onset sepsis in a national cohort of very low birth weight infants. Pediatrics 2010; 125: e736-740.
11. Dammann O. Inflammation and retinopathy of prematurity. Acta paediatrica 2010; 99:975-977.
12. Hong H K. Lec H J. Ko J H et al. Neonatal systemic inflammation in rats alters retinal vessel development and simulates pathologic features of retinopathy of prematurity. J Neuroinflammation 2014; 11:87.
13. Dammann O. Brinkhaus M J. Bartels D B et al. Immaturity. perinatal inflammation, and retinopathy of prematurity: a multi-hit hypothesis. Early Hum Dev 2009; 85:325-329.
14. Ohnell H M. Andreasson S. Granse L. Dexamethasone Eye Drops for the Treatment of Retinopathy of Prematurity. Ophthalmol Retina 2022; 6:181-182.
15. Liebowitz M. Clyman R I. Prophylactic Indomethacin Compared with Delayed Conservative Management of the Patent Ductus Arteriosus in Extremely Preterm Infants: Effects on Neonatal Outcomes. J Pediatr 2017; 187:119-126 e111.
16. Irmesi R. Marcialis M A. Anker J V. Fanos V. Non-steroidal anti-inflammatory drugs (NSAIDs) in the management of patent ductus arteriosus (PDA) in preterm infants and variations in attitude in clinical practice: a flight around the world. Curr Med Chem 2014; 21:3132-3152.
17. Kambhampati S P. Clunies-Ross A J. Bhutto I et al. Systemic and Intravitreal Delivery of Dendrimers to Activated Microglia/Macrophage in Ischemia/Reperfusion Mouse Retina. Investigative ophthalmology & visual science 2015; 56:4413-4424.
18. Grunin M. Hagbi-Levi S. Chowers I. The role of monocytes and macrophages in age-related macular degeneration. Advances in experimental medicine and biology 2014; 801:199-205.
19. Chambers S E. O'Neill C L. O'Doherty™. Medina R J. Stitt A W. The role of immune-related myeloid cells in angiogenesis. Immunobiology 2013; 218:1370-1375.
20. Mitamura Y. Harada C. Harada T. Role of cytokines and trophic factors in the pathogenesis of diabetic retinopathy. Curr Diabetes Rev 2005; 1:73-81.
21. Tsutsumi C. Sonoda K H, Egashira K et al. The critical role of ocular-infiltrating macrophages in the development of choroidal neovascularization. Journal of leukocyte biology 2003; 74:25-32.
22. Abcouwer S F. Neural inflammation and the microglial response in diabetic retinopathy. J Ocul Biol Dis Infor 2011; 4:25-33.
23. Rashid K. Akhtar-Schaefer I. Langmann T. Microglia in Retinal Degeneration. Frontiers in immunology 2019; 10:1975.
24. Kinuthia U M. Wolf A. Langmann T. Microglia and Inflammatory Responses in Diabetic Retinopathy. Frontiers in immunology 2020; 11:564077.
25. Checchin D. Sennlaub F. Levavasseur E. Leduc M. Chemtob S. Potential role of microglia in retinal blood vessel formation. Investigative ophthalmology & visual science 2006; 47:3595-3602.
26. Zhou Y. Yoshida S. Nakao S et al. M2 Macrophages Enhance Pathological Neovascularization in the Mouse Model of Oxygen-Induced Retinopathy. Investigative ophthalmology & visual science 2015; 56:4767-4777.
27. Gao X. Wang Y S. Li X Q et al. Macrophages promote vasculogenesis of retinal neovascularization in an oxygen-induced retinopathy model in mice. Cell and tissue research 2016; 364:599-610.
28. Kataoka K. Nishiguchi K M. Kaneko H. van Rooijen N. Kachi S. Terasaki H. The roles of vitreal macrophages and circulating leukocytes in retinal neovascularization. Investigative ophthalmology & visual science 2011; 52:1431-1438.
29. Stefater J A. 3rd. Ren S. Lang R A. Duffield J S. Metchnikoff's policemen: macrophages in development. homeostasis and regeneration. Trends Mol Med 2011; 17:743-752.
30. Stefater J A. 3rd. Lewkowich I. Rao S et al. Regulation of angiogenesis by a non-canonical Wnt-Flt 1 pathway in myeloid cells. Nature 2011; 474:511-515.
31. Lang R A. Bishop J M. Macrophages are required for cell death and tissue remodeling in the developing mouse eye. Cell 1993; 74:453-462.
32. Ritter M R. Banin E. Moreno S K. Aguilar E. Dorrell M I. Friedlander M. Myeloid progenitors differentiate into microglia and promote vascular repair in a model of ischemic retinopathy. The Journal of clinical investigation 2006; 116:3266-3276.
33. Binet F. Mawambo G. Sitaras N et al. Neuronal E R stress impedes myeloid-cell-induced vascular regeneration through IRElalpha degradation of netrin-1. Cell metabolism 2013; 17:353-371.
34. Davies M H. Eubanks J P. Powers M R. Microglia and macrophages are increased in response to ischemia-induced retinopathy in the mouse retina. Molecular vision 2006; 12:467-477.
35. Santos A M. Calvente R. Tassi M et al. Embryonic and postnatal development of microglial cells in the mouse retina. The Journal of comparative neurology 2008; 506:224-239.
36. Chen L. Yang P. Kijlstra A. Distribution, markers, and functions of retinal microglia. Ocul Immunol Inflamm 2002; 10:27-39.
37. Yang P. de Vos A F. Kijlstra A. Macrophages and MHC class I I positive cells in the choroid during endotoxin induced uveitis. Br J Ophthalmol 1997; 81:396-401.
38. McMenamin P G. Dendritic cells and macrophages in the uveal tract of the normal mouse eye. Br J Ophthalmol 1999; 83:598-604.
39. Forrester J V. Xu H. Kuffova L. Dick A D. McMenamin P G. Dendritic cell physiology and function in the eye. Immunological reviews 2010; 234:282-304.
40. Qin H. Holdbrooks A T. Liu Y. Reynolds S L. Yanagisawa L L. Benveniste E N. SOCS3 deficiency promotes M I macrophage polarization and inflammation. Journal of immunology 2012; 189:3439-3448.
41. Spence S. Fitzsimons A. Boyd C R et al. Suppressors of cytokine signaling 2 and 3 diametrically control macrophage polarization. Immunity 2013; 38:66-78.
42. Arnold C E. Whyte C S. Gordon P. Barker R N. Rees A J. Wilson H M. A critical role for SOCS3 in promoting M I macrophage activation and function in vitro and in vivo. Immunology 2013.
43. Stahl A. Joyal J S. Chen J et al. SOCS3 is an endogenous inhibitor of pathologic angiogenesis. Blood 2012; 120:2925-2929.
44. Sun Y. Ju M. Lin Z et al. SOCS3 in retinal neurons and glial cells suppresses VEGF signaling to prevent pathological neovascular growth. Sci Signal 2015; 8: ra94.
45. Sun Y. Liu C H. SanGiovanni J P et al. Nuclear receptor RORalpha regulates pathologic retinal angiogenesis by modulating SOCS3-dependent inflammation. Proceedings of the National Academy of Sciences of the United States of America 2015; 112:10401-10406. 40)
46. Hombrebueno J R. Lynch A. Byrne E M et al. Hyaloid Vasculature as a Major Source of STAT3 (+) (Signal Transducer and Activator of Transcription 3) Myeloid Cells for Pathogenic Retinal Neovascularization in Oxygen-Induced Retinopathy. Arteriosclerosis. thrombosis, and vascular biology 2020; 40: e367-e379. 45
47. Ashkar S. Weber G F. Panoutsakopoulou V et al. Eta-1 (osteopontin): an early component of type-1 (cell-mediated) immunity. Science 2000; 287:860-864.
48. Schlecht A. Zhang P. Wolf J et al. Secreted Phosphoprotein 1 Expression in Retinal Mononuclear Phagocytes Links Murine to Human Choroidal Neovascularization. Front Cell Dev Biol 2020; 8:618598.
49. Wang T. Zhou P. Xie X et al. Myeloid lineage contributes to pathological choroidal neovascularization formation via SOCS3. EBioMedicine 2021; 73:103632.
50. Chiang M F. Quinn G E. Fielder A R et al. International Classification of Retinopathy of Prematurity. Third Edition. Ophthalmology 2021; 128: e51-c68.
51. Macosko E Z. Basu A. Satija R et al. Highly Parallel Genome-wide Expression Profiling of Individual Cells Using Nanoliter Droplets. Cell 2015; 161:1202-1214.
52. La Manno G. Soldatov R. Zeisel A et al. RNA velocity of single cells. Nature 2018; 560:494-498.
53. Jin S. Guerrero-Juarez C F. Zhang L et al. Inference and analysis of cell-cell communication using CellChat. Nature communications 2021; 12:1088.
54. Conaway E A. de Oliveira D C. McInnis C M. Snapper S B. Horwitz B H. Inhibition of Inflammatory Gene Transcription by IL-10 Is Associated with Rapid Suppression of Lipopolysaccharide-Induced Enhancer Activation. Journal of immunology 2017; 198:2906-2915.
55. Consortium E P. An integrated encyclopedia of DNA elements in the human genome. Nature 2012; 489:57-74.
56. Wu L H. Lin C. Lin H Y et al. Naringenin Suppresses Neuroinflammatory Responses Through Inducing Suppressor of Cytokine Signaling 3 Expression. Mol Neurobiol 2016; 53:1080-1091.
57. Wiejak J. Dunlop J. Mackay S P. Yarwood S J. Flavanoids induce expression of the suppressor of cytokine signalling 3 (SOCS3) gene and suppress IL-6-activated signal transducer and activator of transcription 3 (STAT3) activation in vascular endothelial cells. The Biochemical journal 2013; 454:283-293.
58. Srivastava A. Yadav T. Sharma S. Nayak A. Kumari A A. Mishra N. Polymers in drug delivery. Journal of Biosciences and Medicines 2015; 4:69-84.
59. He C. Liu Y. Huang Z et al. A specific RIP3 (+) subpopulation of microglia promotes retinopathy through a hypoxia-triggered necroptotic mechanism. Proceedings of the National Academy of Sciences of the United States of America 2021; 118.
60. Liu Z. Shi H. Xu J et al. Single-cell transcriptome analyses reveal microglia types associated with proliferative retinopathy. JCI Insight 2022; 7.
61. Quinn G E. Schaffer D B. Johnson L. A revised classification of retinopathy of prematurity. American journal of ophthalmology 1982; 94:744-749.
62. Deczkowska A. Keren-Shaul H. Weiner A. Colonna M. Schwartz M. Amit I. Disease-Associated Microglia: A Universal Immune Sensor of Neurodegeneration. Cell 2018; 173:1073-1081.
63. Keren-Shaul H. Spinrad A. Weiner A et al. A Unique Microglia Type Associated with Restricting Development of Alzheimer's Disease. Cell 2017; 169:1276-1290 e1217.
64. Duan X. Qiao M. Bei F. Kim I J. He Z. Sanes J R. Subtype-specific regeneration of retinal ganglion cells following axotomy: effects of osteopontin and mTOR signaling. Neuron 2015; 85:1244-1256.
65. Li S. Jakobs T C. Secreted phosphoprotein 1 slows neurodegeneration and rescues visual function in mouse models of aging and glaucoma. Cell Rep 2022; 41:111880.
66. Tu W. Zheng H. Li L et al. Secreted phosphoprotein 1 promotes angiogenesis of glioblastoma through upregulating PSMA expression via transcription factor HIF-1alpha. Acta biochimica ct biophysica Sinica 2022.
67. Bai Q. Wang X. Yan H et al. Microglia-Derived Spp1 Promotes Pathological Retinal Neovascularization via Activating Endothelial Kit/Akt/mTOR Signaling. J Pers Med 2023; 13.
68. Wang D. Yamamoto S. Hijiya N. Benveniste E N. Gladson C L. Transcriptional regulation of the human osteopontin promoter: functional analysis and DNA-protein interactions. Oncogene 2000; 19:5801-5809.
69. Wai P Y. Mi Z. Gao C. Guo H. Marroquin C. Kuo P C. Ets-1 and runx2 regulate transcription of a metastatic gene, osteopontin. in murine colorectal cancer cells. The Journal of biological chemistry 2006; 281:18973-18982.
70. Schultz J. Lorenz P. Ibrahim S M. Kundt G. Gross G. Kunz M. The functional-443T/C osteopontin promoter polymorphism influences osteopontin gene expression in melanoma cells via binding of c-Myb transcription factor. Molecular carcinogenesis 2009; 48:14-23.
71. Liu Y N. Kang B B. Chen J H. Transcriptional regulation of human osteopontin promoter by C/EBPalpha and AML-1 in metastatic cancer cells. Oncogene 2004; 23:278-288.
72. Grubman A. Choo X Y. Chew G et al. Transcriptional signature in microglia associated with Abeta plaque phagocytosis. Nature communications 2021; 12:3015.
73. Madonna S. Scarponi C. Morelli M et al. SOCS3 inhibits the pathological effects of IL-22 in non-melanoma skin tumor-derived keratinocytes. Oncotarget 2017; 8:24652-24667.
74. La Manna S. Lee E. Ouzounova M et al. Mimetics of suppressor of cytokine signaling 3: Novel potential therapeutics in triple breast cancer. International Journal of Cancer 2018; 143:2177-2186.
75. La Manna S. Lopez-Sanz L. Mercurio F A et al. Chimeric peptidomimetics of SOCS 3 able to interact with JAK2 as anti-inflammatory compounds. ACS medicinal chemistry letters 2020; 11:615-623.
76. La Manna S. Leone M. Mercurio F A. Florio D. Marasco D. Structure-Activity Relationship Investigations of Novel Constrained Chimeric Peptidomimetics of SOCS3 Protein Targeting JAK2. Pharmaceuticals 2022; 15:458.
77. Beguier F. Housset M. Roubeix C et al. The 10q26 Risk Haplotype of Age-Related Macular Degeneration Aggravates Subretinal Inflammation by Impairing Monocyte Elimination. Immunity 2020; 53:429-441 c428.
78. Fujita N. Fujita S. Ogata N et al. Endogenous osteopontin involvement in laser-induced choroidal neovascularization in mice. Investigative ophthalmology & visual science 2011; 52:9310-9315.
79. Yasukawa H. Ohishi M. Mori H et al. IL-6 induces an anti-inflammatory response in the absence of SOCS3 in macrophages. Nature immunology 2003; 4:551-556.
80. Reed A S. Unger E K. Olofsson L E. Piper M L. Myers M G. Jr., Xu A W. Functional role of suppressor of cytokine signaling 3 upregulation in hypothalamic leptin resistance and long-term energy homeostasis. Diabetes 2010; 59:894-906.
81. Smith L E. Wesolowski E. Mclellan A et al. Oxygen-induced retinopathy in the mouse. Investigative ophthalmology & visual science 1994; 35:101-111.
82. Connor K M. Krah N M. Dennison R J et al. Quantification of oxygen-induced retinopathy in the mouse: a model of vessel loss, vessel regrowth and pathological angiogenesis. Nat Protoc 2009; 4:1565-1573.
83. Xiao S. Bucher F. Wu Y et al. Fully automated, deep learning segmentation of oxygen-induced retinopathy images. JCI Insight 2017; 2.
84. Stahl A. Chen J. Sapicha P et al. Postnatal weight gain modifies severity and functional outcome of oxygen-induced proliferative retinopathy. The American journal of pathology 2010; 177:2715-2723.
85. Hao Y. Hao S. Andersen-Nissen E et al. Integrated analysis of multimodal single-cell data. Cell 2021; 184:3573-3587 e3529.
86. Cai G. Yu X. Youn C. Zhou J. Xiao F. SCANNER: a web platform for annotation. visualization and sharing of single cell RNA-seq data. Database (Oxford) 2022; 2022.
87. Borcherding N. Vishwakarma A. Voigt A P et al. Mapping the immune environment in clear cell renal carcinoma by single-cell genomics. Commun Biol 2021; 4:122.
88. Subramanian A. Tamayo P. Mootha V K et al. Gene set enrichment analysis: a knowledge-based approach for interpreting genome-wide expression profiles. Proceedings of the National Academy of Sciences of the United States of America 2005; 102:15545-15550.
89. https://www.gsea-msigdb.org/gsea/msigdb/.
90. https://www.gencodegenes.org/.
91. Bergen V. Lange M. Peidli S. Wolf F A. Theis F J. Generalizing RNA velocity to transient cell states through dynamical modeling. Nat Biotechnol 2020; 38:1408-1414.
92. https://github.com/sqjin/CellChat/blob/master/tutorial/CellChat-vignette.html.
93. Gong Y. Li J. Sun Y et al. Optimization of an Image-Guided Laser-Induced Choroidal Neovascularization Model in Mice. PloS one 2015; 10:00132643.

TABLE 1
Software and Algorithms used in this study.

| | | |
|---|---|---|
| Cell Ranger | 10x Genomics | 3.1.0 |
| R | r-project.org/ | 4.2.1 |
| RStudio | posit.co/ | 2022.07.2 |
| | | Build 576 |
| biomaRt | bioconductor.org/packages/release/bioc/html/biomaRt.html | 2.48.3 |
| CellChat | github.com/sqjin/CellChat | 1.5.0 |
| cowplot | cran.r-project.org/web/packages/cowplot/ | 1.1.1 |
| dittoSeq | bioconductor.org/packages/release/bioc/html/dittoSeq.html | 1.4.4 |
| dplyr | dplyr.tidyverse.org/ | 1.08 |
| Enhanced | bioconductor.org/packages/release/bioc/html/EnhancedVolcano.html | 1.10.0 |
| Volcano | | |
| escape | github.com/ncborcherding/escape | 1.2.0 |
| ggplot2 | ggplot2.tidyverse.org/ | 3.3.5 |
| GSEABase | bioconductor.org/packages/release/bioc/html/GSEABase.html | 1.54.0 |
| htmlwidgets | www.htmlwidgets.org/ | 1.5.4 |
| Matrix | cran.r-project.org/web/packages/Matrix/index.html | 1.3-4 |
| patchwork | patchwork.data-imaginist.com/ | 1.1.1 |
| plotly | plotly.com/r/ | 4.10.0 |
| reticulate | rstudio.github.io/reticulate/ | 1.24 |
| Seurat | satijalab.org/seurat/ | 3.0.0 / 4.1.0 |
| SeuratDisk | github.com/mojaveazure/seurat-disk | 0.0.0.9019 |
| Seurat | github.com/satijalab/seurat-wrappers | 0.3.0 |
| Wrappers | | |
| Python | www.python.org/ | 3.8 |
| Jupyter | jupyter.org/ | 6.4.11 |
| Notebook | | |
| scanpy | scanpy.readthedocs.io/en/stable/ | 1.9.1 |
| scFates | scfates.readthedocs.io/en/latest/index.html | 0.9.0 |
| anndata | anndata.readthedocs.io/en/latest/ | 0.8.0 |
| numpy | numpy.org/ | 1.21.6 |
| pandas | pandas.pydata.org/ | 1.1.5 |
| palantir | palantir.com/docs/foundry/transforms-python/use-python-libraries/ | 1.0.1 |
| matplotlib | matplotlib.org/ | 3.5.1 |
| seaborn | seaborn.pydata.org/ | 0.11.2 |
| scvelo | scvelo.readthedocs.io/en/stable/ | 0.2.4 |

TABLE 2
Primers for RT-PCR.

| Primer | | SEQ |
|---|---|---|
| names | Sequence (5′ to 3′) | ID NO: |
| Spp1_Forward | AGCAAGAAACTCTTCCAAGCAA | 1 |
| Spp1_Reverse | GTGAGATTCGTCAGATTCATCCG | 2 |
| Cyclophilin | CAGACGCCACTGTCGCTTT | 3 |
| A_F | | |
| Cyclophilin | TGTCTTTGGAACTTTGTCTGCAA | 4 |
| A_R | | |

TABLE 3
Primers for Luciferase reporters.

| Primer | | SEQ |
|---|---|---|
| names | Sequence (5′ to 3′) | ID NO: |
| Spp1_E1_F | TAAGCTAGCCCAACAACTTCACGGGATG | 5 |
| Spp1_E1_R | TTCAGATCTAAGTGCATGTGGAGGCCAGAG | 6 |
| Spp1_E2_F | TAAGCTAGCACAACACCAGATCATTACAATG | 7 |
| Spp1_E2_R | AATCTCGAGCCTATGTGTGCAGGCTACC | 8 |
| Spp1_P2.6K_ | GTGACGCGTATCTAACTTCTTACAGTATTTACATTTT | 9 |
| F | AAG | |
| Spp1_P2.6K_ | TTCAGATCTCAGACCTCCCAGAATTTAAATG | 10 |
| R | | |
| Spp1_P2.6K | TGAGAAATACTTCCTTACTCGGCAGTGTGGCACACA | 11 |
| mut-1_F | GGGTGCATACAGTCG | |
| Spp1_P2.6K | CGACTGTATGCACCCTGTGTGCCACACTGCCGAGTA | 12 |
| mut-1_R | AGGAAGTATTTCTCA | |
| Spp1_P2.6K | TGCTGCCTTAGGATTTGTGGAATGTGCCTCACCAAC | 13 |
| mut-2_F | ACTGAAAACACAAACTCC | |
| Spp1_P2.6K | GGAGTTTGTGTTTTCAGTGTTGGTGAGGCACATTCCA | 14 |
| mut-2_R | CAAATCCTAAGGCAGCA | |
| Spp1_P2.6K | TACAAATTTTAAAATCTTTTTAAGTAGCCAACCTTAT | 15 |
| mut-3_F | ATAGTGCAGCACATAAAACTCAAAAAAGATAATTCA | |
| | GAAATATAAGCTGTG | |
| Spp1_P2.6K | CACAGCTTATATTTCTGAATTATCTTTTTTGAGTTTTA | 16 |
| mut-3_R | TGTGCTGCACTATATAAGGTTGGCTACTTAAAAAGA | |
| | TTTTAAAATTTGTA | |
| Spp1_P2.6K | CAAAAAAGATAATTCAGAAATATAAGCTGTGTCTTT | 17 |
| mut-4_F | AAAAAAATAGTGAGACACCCCTTTAACGTGTACAGC | |
| | TTAAAG | |
| Spp1_P2.6K | CTTTAAGCTGTACACGTTAAAGGGGTGTCTCACTATT | 18 |
| mut-4_R | TTTTTAAAGACACAGCTTATATTTCTGAATTATCTTT | |
| | TTTG | |

What is claimed is:

1. A method of reducing activation of myeloid lineage cells in a subject, the method comprising administering to a subject in need thereof an effective amount of an agent that results in a decrease in the expression and/or activity of a factor associated with myeloid cell activation.

2. The method of claim 1, wherein the factor associated with myeloid cell activation is selected from the group consisting of secreted phosphoprotein 1 (Spp1), triggering receptor expressed on myeloid cells 2 (Trem2), translocator protein (Tspo), and signal transducer and activator of transcription 3 (STAT3), or a combination thereof.

3. The method of claim 1 or 2, wherein the agent inhibits expression of the factor associated with myeloid cell activation.

4. The method of claim 3, wherein the agent comprises a small interfering RNA (siRNA), a short hairpin RNA (shRNA), or an antisense oligonucleotide (ASO) that is complementary to a gene encoding the factor associated with myeloid cell activation.

5. The method of claim 3, wherein the agent comprises a viral vector that encodes a siRNA or a shRNA that is complementary to a gene encoding the factor associated with myeloid cell activation.

6. The method of claim 5, wherein the viral vector is a lentiviral vector or a recombinant adeno-associated viral vector (rAAV).

7. The method of any one of claims 4-6, wherein the siRNA, shRNA, or ASO is at least 60%, at least 70%, at least 80%, at least 90%, at least 95%, at least 99%, or 100% complementary to a region of the gene encoding the factor associated with myeloid cell activation.

8. The method of claim 1 or 2, wherein the agent inhibits the activity of the factor associated with myeloid cell activation.

9. The method of claim 8, wherein the agent is a protein, a peptide, an aptamer, or a small molecule.

10. The method of claim 9, wherein the agent binds to the factor associated with myeloid cell activation.

11. The method of claim 10, wherein the agent is an antibody that is specific for the factor associated with myeloid cell activation.

12. The method of any one of claims 1-11, further comprising administering to the subject a second agent that results in an increase in the expression and/or activity of suppressor of cytokine signaling 3 (SOCS3).

13. The method of any one of claims 1-11, further comprising administering to the subject a second agent that results in a decrease in the expression and/or activity of cellular Fos proto-oncogene (c-Fos) or vascular endothelial growth factor A (VEGFA).

14. The method of claim 12 or 13, wherein the second agent is a RNA, a protein, a peptide, an aptamer, or a small molecule.

15. The method of any one of claims 1-14, wherein the administration results in a decrease in the expression and/or activity of the factor associated with myeloid cell activation by up to 10%, up to 20%, up to 30%, up to 40%, up to 50%, up to 60%, up to 70%, up to 80%, up to 90%, up to 95%, up to 99%, or up to 100%, as compared to expression and/or activity of the factor associated with myeloid cell activation prior to the administration.

16. The method of claim 15, wherein the expression and/or activity of the factor associated with myeloid cell activation is decreased in myeloid lineage cells of the subject.

17. The method of any one of claims 1-16, wherein the administration results in reduced activation of myeloid lineage cells in the subject by up to 10%, up to 20%, up to 30%, up to 40%, up to 50%, up to 60%, up to 70%, up to 80%, up to 90%, up to 95%, up to 99%, or up to 100%, as compared to myeloid lineage cell activation in the absence of the administration.

18. The method of claim 17, wherein the activation of myeloid lineage cells comprises proliferation of myeloid lineage cells in the subject.

19. The method of any one of claims 1-18, wherein the myeloid lineage cells are myeloid lineage cells occurring in the retina of the subject.

20. The method of any one of claims 1-19, wherein the myeloid lineage cells are microglial cells, macrophages, B cells, T cells, monocytes, or granulocytes.

21. The method of any one of claims 1-20, wherein the myeloid lineage cells are myeloid lineage cells in which expression and/or activity of Spp1, Trem2, Tspo, and/or STAT3 is upregulated prior to administration.

22. The method of any one of claims 1-21, wherein the myeloid lineage cells are myeloid lineage cells in which expression and/or activity of SOCS3 is downregulated prior to administration.

23. The method of any one of claims 1-22, wherein the administration results in reduced neovascularization in the retina of the subject by up to 10%, up to 20%, up to 30%, up to 40%, up to 50%, up to 60%, up to 70%, up to 80%, up to 90%, up to 95%, up to 99%, or up to 100%, as compared to neovascularization in the retina of the subject in the absence of the administration.

24. The method any one of claims 1-23, wherein the subject is a mammalian subject.

25. The method of claim 24, wherein the subject is a human.

26. The method of any one of claims 1-25, wherein the subject has, is suspected of having, or is at risk of developing a retinopathy, a retinal injury, or a glaucoma associated with retinopathy.

27. The method of claim 26, wherein the retinopathy is retinopathy of prematurity (ROP), dry age-related macular degeneration (AMD), wet AMD, retinal degeneration, retinitis pigmentosa, or diabetic retinopathy.

28. The method of claim 26 or 27, wherein the administration treats the retinopathy, retinal injury, or glaucoma associated with retinopathy in the subject.

29. The method of claim 26 or 27, wherein the administration prevents the development of the retinopathy, retinal injury, or glaucoma associated with retinopathy in the subject.

30. A method of treating a retinopathy, a retinal injury, or a glaucoma associated with retinopathy in a subject, the method comprising:
(a) collecting myeloid lineage cells from the subject in need thereof;
(b) treating the myeloid lineage cells with an agent that results in a decrease in the expression and/or activity of a factor associated with myeloid cell activation; and
(c) administering the myeloid lineage cells to the subject, thereby treating a retinopathy, a retinal injury, or a glaucoma associated with retinopathy in the subject.

31. The method of claim 30, wherein the factor associated with myeloid cell activation is selected from the group consisting of secreted phosphoprotein 1 (Spp1), triggering receptor expressed on myeloid cells 2 (Trem2), translocator protein (Tspo), and signal transducer and activator of transcription 3 (STAT3), or a combination thereof.

32. The method of claim 30 or 31, wherein the agent inhibits expression of the factor associated with myeloid cell activation.

33. The method of claim 32, wherein the agent comprises a small interfering RNA (siRNA), a short hairpin RNA (shRNA), or an antisense oligonucleotide (ASO) that is complementary to a gene encoding the factor associated with myeloid cell activation.

34. The method of claim 32, wherein the agent comprises a viral vector that encodes a siRNA or a shRNA that is complementary to a gene encoding the factor associated with myeloid cell activation.

35. The method of claim 34, wherein the viral vector is a lentiviral vector or a recombinant adeno-associated viral vector (rAAV).

36. The method of any one of claims 33-35, wherein the siRNA, shRNA, or ASO is at least 60%, at least 70%, at least 80%, at least 90%, at least 95%, at least 99%, or 100% complementary to a region of the gene encoding the factor associated with myeloid cell activation.

37. The method of claim 30 or 31, wherein the agent inhibits the activity of the factor associated with myeloid cell activation.

38. The method of claim 37, wherein the agent is a protein, a peptide, an aptamer, or a small molecule.

39. The method of claim 38, wherein the agent binds to the factor associated with myeloid cell activation.

40. The method of claim 39, wherein the agent is an antibody that is specific for the factor associated with myeloid cell activation.

41. The method of any one of claims 30-40, further comprising treating the myeloid lineage cells in (b) with a second agent that results in an increase in the expression and/or activity of suppressor of cytokine signaling 3 (SOCS3).

42. The method of any one of claims 30-40, further comprising treating the myeloid lineage cells in (b) with a second agent that results in a decrease in the expression and/or activity of cellular Fos proto-oncogene (c-Fos) or vascular endothelial growth factor A (VEGFA).

43. The method of claim 41 or 42, wherein the second agent is a RNA, a protein, a peptide, an aptamer, or a small molecule.

44. The method of any one of claims 30-43, wherein the treatment in (b) results in a decrease in the expression and/or activity of the factor associated with myeloid cell activation in the myeloid lineage cells by up to 10%, up to 20%, up to 30%, up to 40%, up to 50%, up to 60%, up to 70%, up to 80%, up to 90%, up to 95%, up to 99%, or up to 100%, as compared to expression and/or activity of the factor associated with myeloid cell activation prior to the treatment.

45. The method of any one of claims 30-44, wherein the treatment in (b) results in reduced activation of the myeloid lineage cells by up to 10%, up to 20%, up to 30%, up to 40%, up to 50%, up to 60%, up to 70%, up to 80%, up to 90%, up to 95%, up to 99%, or up to 100%, as compared activation of the myeloid lineage cells prior to the treatment.

46. The method of claim 45, wherein the activation of the myeloid lineage cells comprises proliferation of the myeloid lineage cells.

47. The method of any one of claims 30-46, wherein the myeloid lineage cells are microglial cells, macrophages, B cells, T cells, monocytes, or granulocytes.

48. The method of any one of claims 30-47, wherein in (a) the myeloid lineage cells are collected from bone marrow of the subject.

49. The method of any one of claims 30-48, wherein in (c) the myeloid lineage cells are administered to the retina of the subject.

50. The method of claim 49, wherein the administration in (c) results in reduced neovascularization in the retina of the subject by up to 10%, up to 20%, up to 30%, up to 40%, up to 50%, up to 60%, up to 70%, up to 80%, up to 90%, up to 95%, up to 99%, or up to 100%, as compared to neovascularization in the retina of the subject in the absence of the administration.

51. The method any one of claims 30-50, wherein the subject is a mammalian subject.

52. The method of claim 51, wherein the subject is a human.

53. The method of any one of claims 30-52, wherein the retinopathy is retinopathy of prematurity (ROP), dry age-related macular degeneration (AMD), wet AMD, retinal degeneration, retinitis pigmentosa, or diabetic retinopathy.


SEQUENCE LISTING (21 sequences)

SEQ ID NO: 1 - DNA, 22 bp, synthetic construct
  source 1..22: mol_type = other DNA
agcaagaaactcttccaagcaa

SEQ ID NO: 2 - DNA, 23 bp, synthetic construct
  source 1..23: mol_type = other DNA
gtgagattcgtcagattcatccg

SEQ ID NO: 3 - DNA, 19 bp, synthetic construct
  source 1..19: mol_type = other DNA
cagacgccactgtcgcttt

SEQ ID NO: 4 - DNA, 23 bp, synthetic construct
  source 1..23: mol_type = other DNA
tgtctttggaactttgtctgcaa

SEQ ID NO: 5 - DNA, 28 bp, synthetic construct
  source 1..28: mol_type = other DNA
taagctagcccaacaacttcacgggatg

SEQ ID NO: 6 - DNA, 30 bp, synthetic construct
  source 1..30: mol_type = other DNA
ttcagatctaagtgcatgtggaggccagag

SEQ ID NO: 7 - DNA, 31 bp, synthetic construct
  source 1..31: mol_type = other DNA
taagctagcacaacaccagatcattacaatg

SEQ ID NO: 8 - DNA, 28 bp, synthetic construct
  source 1..28: mol_type = other DNA
aatctcgagcctatgtgtgcaggctacc

SEQ ID NO: 9 - DNA, 40 bp, synthetic construct
  source 1..40: mol_type = other DNA
gtgacgcgtatctaacttcttacagtatttacattttaag

SEQ ID NO: 10 - DNA, 31 bp, synthetic construct
  source 1..31: mol_type = other DNA
ttcagatctcagacctcccagaatttaaatg

SEQ ID NO: 11 - DNA, 51 bp, synthetic construct
  source 1..51: mol_type = other DNA
tgagaaatacttccttactcggcagtgtggcacacagggtgcatacagtcg

SEQ ID NO: 12 - DNA, 51 bp, synthetic construct
  source 1..51: mol_type = other DNA
cgactgtatgcaccctgtgtgccacactgccgagtaaggaagtatttctca

SEQ ID NO: 13 - DNA, 54 bp, synthetic construct
  source 1..54: mol_type = other DNA
tgctgccttaggatttgtggaatgtgcctcaccaacactgaaaacacaaactcc

SEQ ID NO: 14 - DNA, 54 bp, synthetic construct
  source 1..54: mol_type = other DNA
ggagtttgtgttttcagtgttggtgaggcacattccacaaatcctaaggcagca

SEQ ID NO: 15 - DNA, 88 bp, synthetic construct
  source 1..88: mol_type = other DNA
tacaaattttaaaatctttttaagtagccaaccttatatagtgcagcacataaaactcaaaaaagataattcagaaatataagctgtg

SEQ ID NO: 16 - DNA, 88 bp, synthetic construct
  source 1..88: mol_type = other DNA
cacagcttatatttctgaattatcttttttgagttttatgtgctgcactatataaggttggctacttaaaaagattttaaaatttgta

SEQ ID NO: 17 - DNA, 78 bp, synthetic construct
  source 1..78: mol_type = other DNA
caaaaaagataattcagaaatataagctgtgtctttaaaaaaatagtgagacacccctttaacgtgtacagcttaaag

SEQ ID NO: 18 - DNA, 78 bp, synthetic construct
  source 1..78: mol_type = other DNA
ctttaagctgtacacgttaaaggggtgtctcactatttttttaaagacacagcttatatttctgaattatcttttttg

SEQ ID NO: 19 - protein, 16 aa, synthetic construct
FSSKSEYQLFYWSAVY

SEQ ID NO: 20 - protein, 10 aa, synthetic construct
AAARAARAAA

SEQ ID NO: 21 - protein, 14 aa, synthetic construct
GRKKRRQRRRPQGG